(19) United States
(12) Patent Application Publication
(10) Pub. No.: US 2015/0169834 A1
(43) Pub. Date: Jun. 18, 2015

(54) FATIGUE LEVEL ESTIMATION METHOD, PROGRAM, AND METHOD FOR PROVIDING PROGRAM

(71) Applicant: Panasonic Intellectual Property Corporation of America, Torrance, CA (US)

(72) Inventors: Kozo Nishimura, Osaka (JP); Tomohiko Kitamura, Osaka (JP); Kouji Miura, Osaka (JP); Shunji Harada, Osaka (JP)

(21) Appl. No.: 14/403,743

(22) PCT Filed: Mar. 19, 2014
(86) PCT No.: PCT/JP2014/001593

§ 371 Date: Nov. 25, 2014

(30) Foreign Application Priority Data
Mar. 25, 2013 (JP) 2013-062855

(51) Int. Cl. G06F 19/00; G01D 9/00; A61B 5/00; A61B 5/16; A61B 5/11

(52) U.S. Cl. CPC G06F 19/345; A61B 5/165; A61B 5/1126; A61B 5/486; A61B 5/7278; A61B 5/7267; A61B 5/7475; G06F 19/3406; G01D 9/005

(57) ABSTRACT

A fatigue degree estimating method analyzes log information including an operation log of a user with respect to a device and a state log of the device for analyzing a direct operation by the user with respect to the device, and movement of the user before and after the direct operation; and estimates the fatigue degree of each part of the user and of a mental part of the user by the analyzed direct operation and the analyzed movement before and after the direct operation. The estimated fatigue degree of each part of the user and of the mental part of the user are notified to the user.

### TECHNICAL FIELD

The present invention relates to a technique of utilizing information such as an operation history and a movement history of household electrical devices such as a washing machine, a cleaner, and an iron for estimating a fatigue degree of a user in doing household chores.

### BACKGROUND ART

Conventionally, in a system for evaluating a fatigue degree of a user with respect to an activity, a fatigue degree is calculated by counting a frequency of activities detected by an activity sensor, and by determining a fatigue level in accordance with the counted frequency of activities.

Patent literature 1 discloses a technique of estimating a fatigue degree of a driver driving an automobile from sensor information relating to a steering operation and a vehicle speed during driving the automobile.

Further, patent literature 2 discloses a technique of determining a physical condition of a user by acquiring the number of times of operating a household electrical device, calculating an entropy value at a predetermined time interval, and on the basis of the user's life activity degree acquired by comparison with a reference entropy.

However, there is a demand for a further investigation in order to enhance the estimation precision of a fatigue degree.

### CITATION LIST

### Patent Literature

Patent literature 1: Japanese Unexamined Patent Publication No. 2005-210361

Patent literature 2: Japanese Patent Publication No. 4,865,046

### SUMMARY OF INVENTION

In view of the above, a fatigue degree estimating method according to an aspect of the invention is a fatigue degree estimating method for use in a fatigue degree estimating system for estimating a fatigue degree of a user in doing household chores. The fatigue degree estimating method includes:

a movement analyzing step of analyzing log information including an operation log of the user with respect to a device and a state log of the device for analyzing a direct operation by the user with respect to the device, and movement of the user before and after the direct operation; and

a fatigue degree estimating step of estimating a fatigue degree of each part of the user and of a mental part of the user by the analyzed direct operation and the analyzed movement before and after the direct operation.

In the above configuration, it is possible to estimate a fatigue degree of the user in doing household chores for each part of the user, considering not only a direct operation by the user with respect to the device, but also movement before and after the direct operation. This makes it possible to provide the user with detailed fatigue information. Further, it is also possible to estimate a fatigue degree of a mental part of the user. This is advantageous in providing the user with a fatigue degree physically and mentally.

### BRIEF DESCRIPTION OF DRAWINGS

FIGS. 1A to 1C are diagrams illustrating an example of an overview of a fatigue degree estimating system embodying the invention;

FIG. 2 is a diagram illustrating a service type (a service provided by a datacenter of the applicant's company) in the embodiment of the invention;

FIG. 3 is a diagram illustrating a service type (a service utilizing IaaS) in the embodiment of the invention;

FIG. 4 is a diagram illustrating a service type (a service utilizing PaaS) in the embodiment of the invention;

FIG. 5 is a diagram illustrating a service type (a service utilizing SaaS) in the embodiment of the invention;

FIG. 6 is a block diagram illustrating an example of a fatigue degree estimating system according to the first embodiment of the invention;

FIG. 7A is a diagram illustrating classification of log information data in the first embodiment of the invention;

FIG. 7B is a diagram illustrating an example of a table, in which the operation contents of the user to be analyzed by an operation analysis unit are classified for each device in the first embodiment of the invention;

FIG. 8 is a diagram illustrating an example of a table, in which the fatigue parts of the user to be estimated by a fatigue degree estimation unit are classified for each device in the first embodiment of the invention;

FIG. 9A is a diagram illustrating an example of a relationship between calculation parameters for use in calculating fatigue degrees by the fatigue degree estimation unit, and fatigue degrees in the first embodiment of the invention;

FIG. 9B is a diagram illustrating an example of a relationship between a calculation parameter and a fatigue degree in the first embodiment of the invention;

FIG. 9C is a diagram illustrating an example of a relationship between a calculation parameter and a fatigue degree in the first embodiment of the invention;

FIG. 9D is a diagram illustrating an example of a relationship between a calculation parameter and a fatigue degree in the first embodiment of the invention;

FIG. 9E is a diagram illustrating an example of a relationship between a calculation parameter and a fatigue degree in the first embodiment of the invention;

FIG. 9F is a diagram illustrating an example of a relationship between a calculation parameter and a fatigue degree in the first embodiment of the invention;

FIG. 9G is a diagram illustrating an example of a relationship between a calculation parameter and a fatigue degree in the first embodiment of the invention;

FIG. 10A is a diagram illustrating an example of fatigue parts to be estimated from washing machine log information in the first embodiment of the invention;

FIG. 10B is a diagram illustrating an example of a table, in which washing machine log information and calculation parameters are mapped to each other in the first embodiment of the invention;

FIG. 11A is a diagram illustrating an example of fatigue parts to be estimated from cleaner log information in the first embodiment of the invention;

FIG. 11B is a diagram illustrating an example of a table, in which cleaner log information and calculation parameters are mapped to each other in the first embodiment of the invention;

FIG. 12A is a diagram illustrating an example of fatigue parts to be estimated from iron log information in the first embodiment of the invention;

FIG. 12B is a diagram illustrating an example of a table, in which iron log information and calculation parameters are mapped to each other in the first embodiment of the invention;

FIG. 13 is a diagram illustrating an example of log information of a user stored in log information DB in the first embodiment of the invention;

FIG. 14 is a diagram illustrating a comparison between this time and ordinary time with respect to washing machine log information in the first embodiment of the invention;

FIG. 15A is a diagram illustrating an example of a table to be used in calculating a “RATIO TO PAST AVERAGE” with respect to a calculation parameter “OPERATION TIME DIFFERENCE” in the first embodiment of the invention;

FIG. 15B is a diagram illustrating an example of a table for use in determining an increment for the fatigue degree in cleaning a high place and in cleaning a low place in the first embodiment of the invention;

FIG. 15C is a diagram illustrating an example of a table for use in determining an increment for the fatigue degree relating to an operation state of a cleaner (a state by which hand the cleaner is operated) in the first embodiment of the invention;

FIG. 15D is a diagram illustrating an example of a table which defines a weighting coefficient of each calculation parameter to be applied to each part in the first embodiment of the invention;

FIG. 15E is a diagram illustrating an example of a table, in which the number of household chores to be performed concurrently, and an increment value are mapped to each other in the first embodiment of the invention;

FIG. 16 is a diagram illustrating an example of a notification image indicating a fatigue degree estimation result to be provided by the fatigue degree estimation unit, which is notified to at least one of the user and a person related to the user in the first embodiment of the invention;

FIG. 17 is a block diagram illustrating an example of a configuration of a fatigue degree estimating system according to the second embodiment of the invention;

FIG. 18A is a flowchart illustrating an example of processing to be performed by a fatigue recovery message generation unit in the second embodiment of the invention;

FIG. 18B is a second half flowchart of FIG. 18A;

FIG. 19A is a diagram illustrating an example of a keyword table on levels in the second embodiment of the invention;

FIG. 19B is a diagram illustrating an example of a keyword table on soothing, appreciation, and praise in the second embodiment of the invention;

FIG. 20 is a diagram illustrating an example of a fatigue degree check image in the second embodiment of the invention;

FIG. 21 is a diagram illustrating an example of a notification image indicating a fatigue degree of a household worker in the second embodiment of the invention;

FIG. 22 is a diagram illustrating an example of a notification image indicating a fatigue degree, when a person related to the household worker checks the fatigue degree of the household worker in the second embodiment of the invention;

FIG. 23 is a block diagram illustrating an example of a configuration of a fatigue degree estimating system according to the third embodiment of the invention;

FIG. 24A is a diagram illustrating an example of a device ability table in the third embodiment of the invention;

FIG. 24B is a diagram illustrating an example of a massage chair setting table in the third embodiment of the invention;

FIG. 25 is a flowchart illustrating an example of processing to be performed by a fatigue recovery tip generation unit in the third embodiment of the invention;

FIG. 26A is a flowchart illustrating an example of fatigue recovery tip presentation processing for each part in the third embodiment of the invention;

FIG. 26B is a flowchart illustrating an example of user interface (UI) processing in a fatigue recovery system in the third embodiment of the invention;

FIG. 27 is a diagram illustrating an example of a fatigue recovery tip image in the third embodiment of the invention;

FIG. 28 is a block diagram illustrating a configuration of a fatigue degree estimating system according to the fourth embodiment of the invention;

FIG. 29 is a flowchart illustrating an example of processing to be performed by a setting information generation unit in the fourth embodiment of the invention;

FIG. 30A is a flowchart illustrating an example of fatigue recovery setting processing for each part in the fourth embodiment of the invention;

FIG. 30B is a flowchart illustrating an example of UI processing in a fatigue recovery system in the fourth embodiment of the invention;

FIG. 31A is a diagram illustrating an example of a fatigue recovery setting image in the fatigue degree estimating system according to the fourth embodiment of the invention;

FIG. 31B is a diagram illustrating an example of a UI image of a massage chair owned by the user in the fourth embodiment of the invention;

FIG. 32 is a block diagram illustrating an example of a configuration of a fatigue degree estimating system according to the fifth embodiment of the invention;

FIG. 33 is a flowchart illustrating an example of processing to be performed by a parameter updating unit in the fifth embodiment of the invention; and

FIG. 34 is a diagram illustrating an example of log information when the user uses a massage chair as a remote controllable device after doing household chores indicated by the washing machine log information and the cleaner log information in FIG. 13 in the fifth embodiment of the invention.

### DESCRIPTION OF EMBODIMENTS

### Findings Based on which the Invention has been Made

Fatigue resulting from household work may be neglected because the household work seems to be a simple work such that a user presses an operation button of a household electrical device, and thereafter just waits for the end of operation of the device. Actually, however, there are many works to be done by hand before and after the user presses the operation button. For instance, before and after operating a washing machine, the user has to put the laundry in and out of the washing machine, dry the laundry, collect the laundry, and fold the laundry. Further, there should be considered mental fatigue other than a physical fatigue, for instance, feeling busy about doing multiple household chores concurrently, and feeling uneasy about doing household chores in a way different from the ordinary pattern of doing the household chores. Conventionally, regarding fatigue resulting from household work, there has not been considered a technical solution for estimating a physical fatigue and mental fatigue before and after an operation, which is not directly included in operation information or a movement history of household electrical devices, with use of the operation information and the movement history.

In order to solve the above problems, a fatigue degree estimating method according to an aspect of the invention is a fatigue degree estimating method for use in a fatigue degree estimating system for estimating a fatigue degree of a user in doing household chores. The fatigue degree estimating method includes:

a movement analyzing step of analyzing log information including an operation log of the user with respect to a device and a state log of the device for analyzing a direct operation by the user with respect to the device, and movement of the user before and after the direct operation; and

a fatigue degree estimating step of estimating a fatigue degree of each part of the user and of a mental part of the user by the analyzed direct operation and the analyzed movement before and after the direct operation.

According to the above configuration, the fatigue degree of each part of the user in doing household chores is estimated, taking into consideration not only a direction operation by the user with respect to a device, but also work of the user before and after the direct operation. This makes it possible to estimate the fatigue degree of the user, taking into consideration work relating to all the household chores. Further, a fatigue degree of a mental part of the user is also estimated. This is advantageous in providing the user with a fatigue degree physically and mentally.

Further, in the above configuration, the fatigue degree estimating method may further include a message notifying step of generating a message of soothing, appreciation, or praise in accordance with the estimated fatigue degree, and notifying at least one of the user and a person related to the user of the message.

A message of soothing, appreciation, or praise for the household chores is notified to the user, who is a household worker. This makes it possible to recover the fatigue of the user from a mental approach.

Further, in the above configuration, the fatigue degree estimating method may further include a tip message notifying step of selecting a fatigue recovery device suitable for recovery of the estimated fatigue degree, with use of device ability information in which a fatigue recovery ability of the each part of the user is defined individually with respect to one or more fatigue recovery devices owned by the user, generating a message proposing a fatigue recovery tip utilizing the selected fatigue recovery device, with use of setting information, of the selected fatigue recovery device, which defines setting contents suitable for the fatigue degree of the each part of the user, and notifying the user of the message.

According to the above configuration, a fatigue recovery device suitable for recovery from fatigue of the user is notified to the user from among the fatigue recovery devices owned by the user. This allows for the user to effectively utilize the fatigue recovery devices owned by the user. This is advantageous in guiding the user for recovery from fatigue in a more rational way.

Further, in the above configuration, the fatigue degree estimating method may further include a setting information generating step of selecting a remote controllable device suitable for recovery of the estimated fatigue degree, with use of device ability information in which a recovery ability of the each part of the user is defined individually with respect to one or more remote controllable devices, selecting setting information suitable for recovery of the estimated fatigue degree, from among one or more setting information in which setting contents of the remote controllable device suitable for a fatigue degree of the each part of the user is defined, and transmitting the selected setting information to the remote controllable device, the remote controllable devices being remote controllable fatigue recovery devices owned by the user.

According to the above configuration, the user is allowed to select a remote controllable device suitable for recovery from fatigue of the user from among the remote controllable devices owned by the user. This allows for the user to recover from fatigue without a cumbersome operation.

Further, in the above configuration, the fatigue degree estimating method may further include a learning step of learning fatigue of the user, with use of log information indicating an operation of the remote controllable device by the user, when the remote controllable device is moved with use of the set setting information.

According to the above configuration, actual fatigue of the user is learned with use of log information of the remote controllable device used by the user for recovery from fatigue. This is advantageous in effectively implementing recovery from fatigue of the user.

Further, in the above configuration, the movement analyzing step may include specifying, out of the log information, log information for which a calculation parameter is set, the specified log information relating to the direct operation and to the movement before and after the direct operation.

Further, in the above configuration, the fatigue degree estimating step may include setting a calculation parameter in accordance with contents of the log information with respect to the specified log information, multiplying the set calculation parameter by a weighting coefficient in accordance with the mental part and the each part of the user, and summing the calculation parameter multiplied by the weighting coefficient individually for the mental part and for the each part of the user so as to calculate a fatigue degree of the mental part and of the each part of the user.

The above configuration is advantageous in accurately calculating a fatigue degree of a mental part and each part of the user.

Further, in the above configuration, the message to be notified to the person related to the user may include an input column in which the person related to the user inputs a message to the user, and

the message notifying step may include containing the input message in the message to be notified to the user, when the message is input in the input column.

According to the above configuration, a message of the person related to the user is notified to the user, who is a household worker. This is advantageous in guiding the user for recovery from fatigue from a mental approach.

Further, in the above configuration, the tip message notifying step may include:

calculating a fatigue recovery margin indicating a margin of the recovery ability of each of the fatigue recovery devices with respect to the estimated fatigue degree,

specifying one or more fatigue recovery devices in the order of decreasing the fatigue recovery margin, and setting an image indicating the specified fatigue recovery device with a size corresponding to a magnitude of the fatigue recovery margin for the each part of the user, and

generating the message, while arranging the images whose sizes are set in the order of decreasing the fatigue recovery margin for the each part of the user.

According to the above configuration, a message in which images of fatigue recovery devices suitable for recovery from fatigue are arranged with different sizes in the order of decreasing the fatigue recovery effect is notified to the user. This allows for the user to recognize the fatigue recovery device having a high fatigue recovery effect at a glance.

Further, in the above configuration, the tip message notifying step may include, when an image indicating the fatigue recovery device is selected by the user, generating the message displaying setting information suitable for recovery from fatigue of the user in the selected fatigue recovery device.

According to the above configuration, a message including setting information of a fatigue recovery device suitable for recovery from fatigue is notified to the user. This allows for the user to recognize the setting information of the fatigue recovery device suitable for recovery from fatigue.

Further, in the above configuration, the setting information generating step may include notifying, on a terminal of the user, a message displayed such that an icon for use in downloading the selected setting information is mapped to an image indicating the selected remote controllable device, and transmitting setting information corresponding to the selected icon to the remote controllable device when selection of the icon by the user is detected.

According to the above configuration, the user is allowed to select only the setting information necessary for the user, and download the selected setting information in the remote controllable device owned by the user. This is advantageous in reducing the consumption amount of a memory of the remote controllable device.

Further, in the above configuration, the fatigue degree may be calculated with use of a weighting coefficient in accordance with the mental part and the each part of the user, and

the learning step may include updating the weighting coefficient in such a manner that a fatigue degree of a part of the user for which recovery from fatigue is performed by the remote controllable device is lowered, when log information indicating the operation indicates an operation of suspending movement of the remote controllable device with use of the setting information during the movement.

The fact that movement of a remote controllable device using setting information is suspended during the movement means that the setting information is too much for the fatigue of the user. In view of the above, in this aspect, the weighting coefficient is updated in such a manner that the fatigue degree of a part for which recovery from fatigue is performed by the remote controllable device is lowered. This makes it possible to accurately estimate the fatigue degree of the user and securely guide the user for recovery from fatigue.

Further, in the above configuration, the fatigue degree may be calculated with use of a weighting coefficient in accordance with the mental part and the each part of the user, and

the learning step may include updating the weighting coefficient in such a manner that a fatigue degree of a specific part of the user increases, when log information indicating the operation indicates input of an addition operation for recovery from fatigue of the specific part after movement of the remote controllable device with use of the setting information is ended.

The fact that the user inputs an additional operation for recovering from fatigue of a specific part after movement of a remote controllable device using setting information is ended means that the setting information is insufficient for recovery from fatigue of the specific part. In view of the above, in this aspect, the weighting coefficient is updated in such a manner that the fatigue degree of the specific part increases. This makes it possible to accurately estimate the fatigue degree of the user and securely guide the user for recovery from fatigue.

The following embodiments are examples of the invention. The numerical values, the shapes, the constituent elements, the steps, and the order of steps described in the following embodiments are examples, and do not limit the gist of the invention. Further, among the constituent elements in the following embodiments, the constituent elements that are not described in independent claims defining the broadest scope are described as optional constituent elements. Further, it is possible to combine each of the contents in all the embodiments.

(Overview of Services to be Provided)

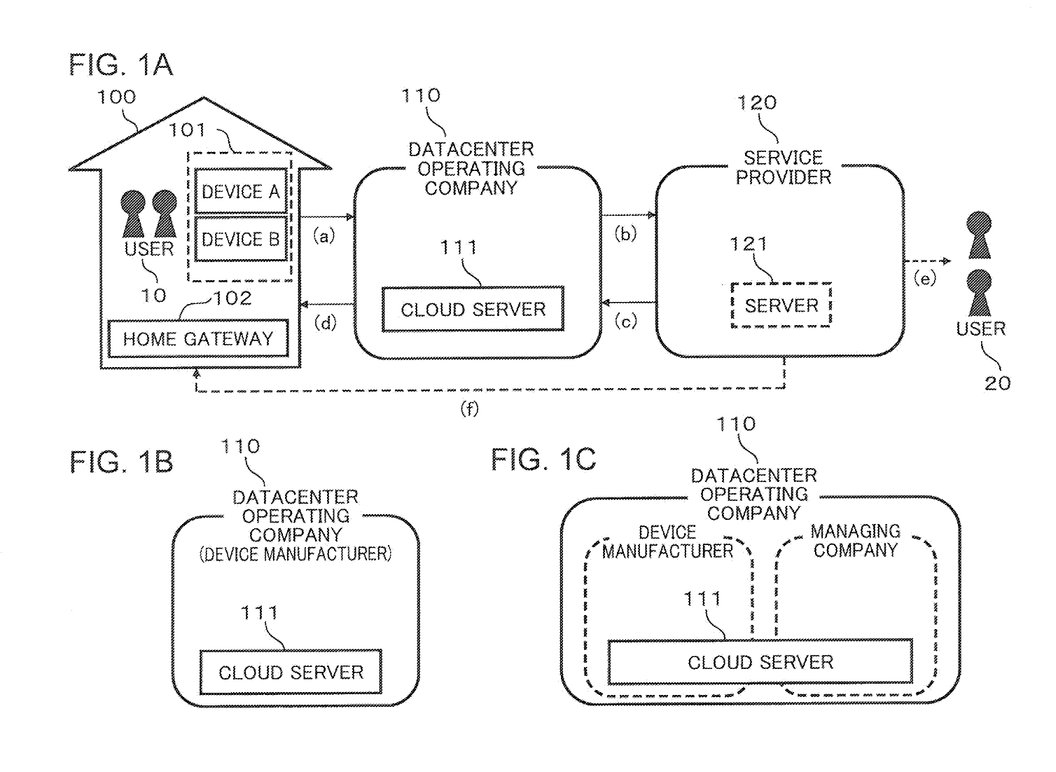

FIG. 1A illustrates an overview of the fatigue degree estimating system embodying the invention.

A group 100 is, for instance, a company, a party, or a home. The scale of the group 100 does not matter. The group 100 is provided with a number of devices 101 including a device A and a device B, and a home gateway 102. The devices 101 include devices (e.g. a smartphone, a PC, a TV receiver) connectable to the Internet, and devices (e.g. an illumination device, a washing machine, a refrigerator) disconnectable to the Internet by themselves. The group 100 may also include devices 101 which are not connectable to the Internet by themselves, but are connectable to the Internet via the home gateway 102. Further, the group 100 also includes users 10 who use the devices 101.

The datacenter operating company 110 is provided with a cloud server 111. The cloud server 111 is a virtual server connectable to a variety of devices via the Internet. The cloud server 111 mainly manages big data, which is difficult to be handled by an ordinary database management tool or the like. The datacenter operating company 110 manages data, manages the cloud server 111, and operates a datacenter which performs these services. The details of the services to be provided by the datacenter operating company 110 will be described later. The datacenter operating company 110 is not limited to a company which manages data or manages the cloud server 111. For instance, when a device manufacturer which develops or manufactures one of the devices 101 manages data or manages the cloud server 111, the device manufacturer corresponds to the datacenter operating company 110 (see FIG. 1B). Further, the number of datacenter operating companies is not limited to one.

For instance, when a device manufacturer and a managing company jointly or sharingly manage data or manage the cloud server 111, both or one of the device manufacturer and the managing company corresponds to the datacenter operating company 110 (see FIG. 1C).

The service provider 120 is provided with a server 121. The scale of the server 121 does not matter. For instance, the server 121 includes a memory in a PC for personal use. Further, the service provider 120 may not be provided with the server 121.

In the aforementioned service, the home gateway 102 is not an essential element. For instance, when the cloud server 111 manages all the data, the home gateway 102 is not necessary. Further, when all the devices in a house are connected to the Internet, a device disconnectable to the Internet by itself may not exist.

Next, a flow of information in the service is described.

The device A or the device B in the group 100 individually transmits log information thereof to the cloud server 111 in the datacenter operating company 110. The cloud server 111 accumulates the log information of the device A or of the device B (see the arrow (a) in FIG. 1A). The log information is information indicating e.g. operation conditions or operation dates and times of the devices 101. For instance, the log information includes a viewing history of TV, video recording reservation information in a recorder, an operation date and time of a washing machine, a quantity of laundry, an opening/closing date and time of a refrigerator, and the number of times of opening/closing a refrigerator. The log information is not limited to the above, and may include all the information acquirable from all the devices. The log information may be directly provided from the devices 101 themselves to the cloud server 111 via the Internet. Further, the log information may be temporarily accumulated in the home gateway 102 from the devices 101, and may be provided from the home gateway 102 to the cloud server 111.

Next, the cloud server 111 in the datacenter operating company 110 provides the accumulated log information to the service provider 120 unit by unit. The unit may correspond to a certain amount of information, by which the datacenter operating company can organize the accumulated information and can provide to the service provider 120, or may correspond to an amount of information required from the service provider 120. In the embodiment, information is provided by a unit. Alternatively, the amount of information to be provided may vary depending on a condition. The log information is stored in the server 121 owned by the service provider 120, as necessary (see the arrow (b) in FIG. 1A).

The service provider 120 organizes the log information into information appropriate for the service to be provided to the user, and provides the organized information to the user. The user to whom information is provided may be a user 10 who uses the devices 101, or may be an outside user 20. The service providing method to the user may be a method, wherein the service is directly provided to the user from the service provider 120 (see the arrows (e) and (f) in FIG. 1A). Further, the service providing method to the user may be a method, wherein the service is provided to the user via the cloud server 111 in the datacenter operating company 110 (see the arrows (c) and (d) in FIG. 1A). Further, the cloud server 111 in the datacenter operating company 110 may organize the log information into information appropriate for the service to be provided to the user, and may provide the organized information to the service provider 120.

The user 10 may be identical to or different from the user 20.

### First Embodiment

### System Configuration of First Embodiment

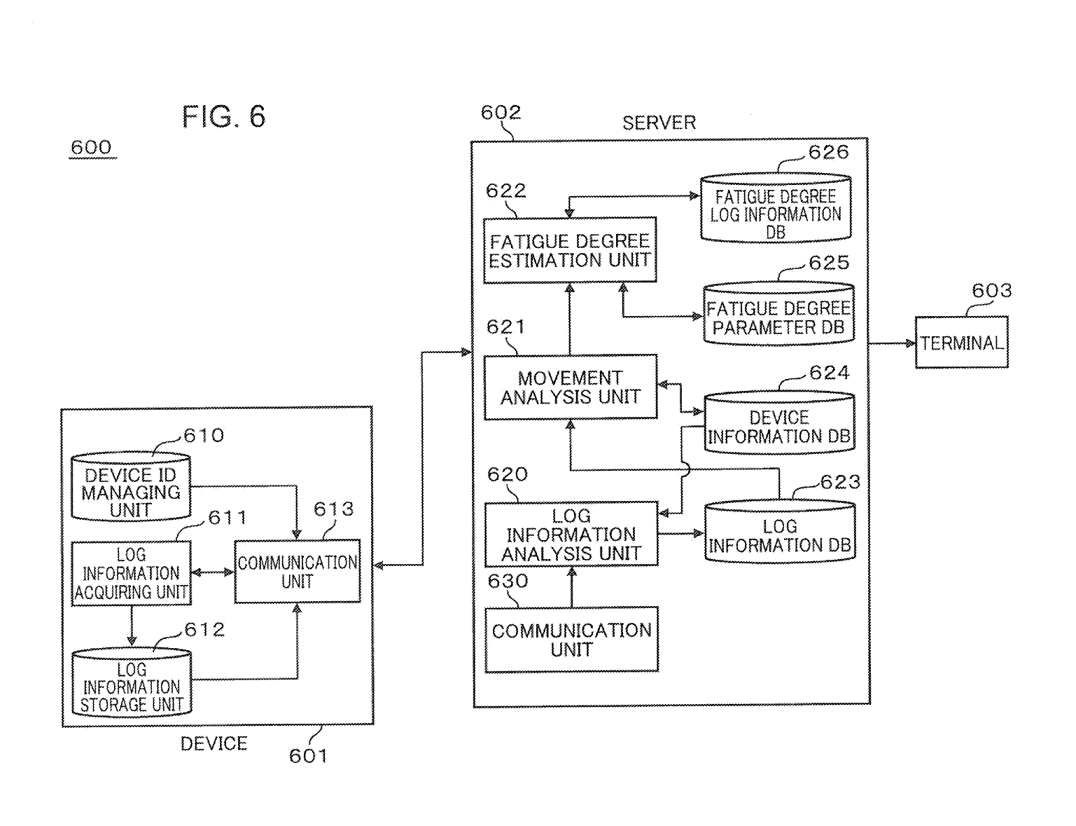

FIG. 6 is a block diagram illustrating an example of a configuration of a fatigue degree estimating system 600 according to the first embodiment of the invention.

As illustrated in FIG. 6, the fatigue degree estimating system 600 is provided with a device 601, a server 602, and a terminal 603. The device 601 is an electrical device to be used by the user when the user does the household chores. Examples of the electrical device are a washing machine, a cleaner, a refrigerator, an IH cooking heater, and a sewing machine.

The device 601 is provided with a device ID managing unit 610, a log information acquiring unit 611, a log information storage unit 612, and a communication unit 613.

The device ID managing unit 610 is constituted of a non-volatile memory such as a flash memory or a HDD (Hard Disk Drive), and is configured to store a unique identifier for identifying the device 601 individually. For instance, the unique identifier may be a serial number of the device 601.

The log information acquiring unit 611 is constituted of a dedicated hardware circuit or a software, and is configured to acquire log information including an operation log relating to a user's operation of the device 601 in doing the household chores and a state log relating to a state of the device 601. When the device is a microwave oven, the log information may be e.g. the code number of a button on the microwave oven pressed by the user, a function identifier as the identifier of a selection function selected by the user, and a heating time of the microwave oven. The log information storage unit 612 is constituted of e.g. a non-volatile memory, and is configured to store log information acquired by the log information acquiring unit 611.

The communication unit 613 is constituted of a communication device for causing the device 601 to connect to a public communication network such as the Internet. The communication unit 613 is configured to transmit the log information stored in the log information storage unit 612 to the server 602 via the Internet in association with the unique identifier stored in the device ID managing unit 610.

The server 602 is a cloud server to be managed by the datacenter operating company 110 or the service provider 120 illustrated in FIGS. 1A to 1C. The server 602 is a virtual server configured to be connected to various devices via the Internet, and is constituted of one or more computers. Further, the server 602 manages big data, which is difficult to be handled by an ordinary database management tool or the like.

The server 602 is provided with a log information analysis unit 620, a movement analysis unit 621, a fatigue degree estimation unit 622, a log information DB 623, a device information DB 624, a fatigue degree parameter DB 625, a fatigue degree log information DB 626, and a communication unit 630.

The communication unit 630 is constituted of e.g. a communication device for causing the server 602 to connect to a public communication network such as the Internet, and is configured to transmit and receive data between the device 601 and the terminal 603. In the embodiment, in particular, the communication unit 630 is configured to receive log information to be transmitted from the device 601, and transmit a fatigue degree estimation result to the terminal 603.

The log information analysis unit 620 is configured to acquire, from the device 601, log information to be transmitted in association with a unique identifier via the communication unit 630. The log information analysis unit 620 is configured to classify the received log information for each user, and store the classified log information in the log information DB 623 in a time-series manner. The log information analysis unit 620 may specify to which user the received log information belongs to by referring to the device DB stored in the device information DB 624, in which the user's identifier and the unique identifier of the device 601 owned by the user are mapped to each other. Further, the log information analysis unit 620 may arrange the log information from the point-of-time information included the log information in a time-series manner.

The movement analysis unit 621 is constituted of e.g. a software or a dedicated hardware circuit, and is configured to analyze a user's direct operation with respect to the device 601, and a user's work (movement) before and after the direct operation, from the log information stored in the log information DB.

The fatigue degree estimation unit 622 is constituted of e.g. a software or a dedicated hardware circuit, and is configured to estimate a fatigue degree of a mental part of the user and of each part of the user from the analysis result of the movement analysis unit 621 in accordance with a fatigue degree parameter stored in the fatigue degree parameter DB 625. The log information DB 623 is a DB (database) which stores an operation sequence and movement information of each device 601. The device information DB 624 is a DB which stores device information of the device 601 of the user whose fatigue degree is estimated. The fatigue degree parameter 625 is a DB which stores calculation parameters for use in calculating a fatigue degree of the user. The fatigue degree log information DB 626 is a DB which stores fatigue degree estimation results of the user.

The terminal 603 is a terminal to be carried by the user to which the service by the fatigue degree estimating system is applied. The terminal 603 may be a mobile information processing device such as a smartphone, a tablet terminal, or a button-type mobile phone, or may be an installation-type information processing device such as a desktop personal computer.

<Analysis of Log Information>

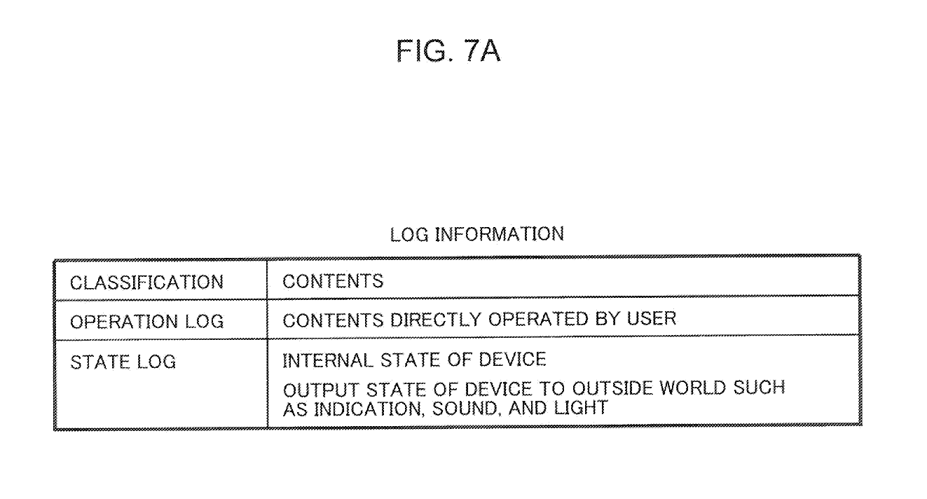

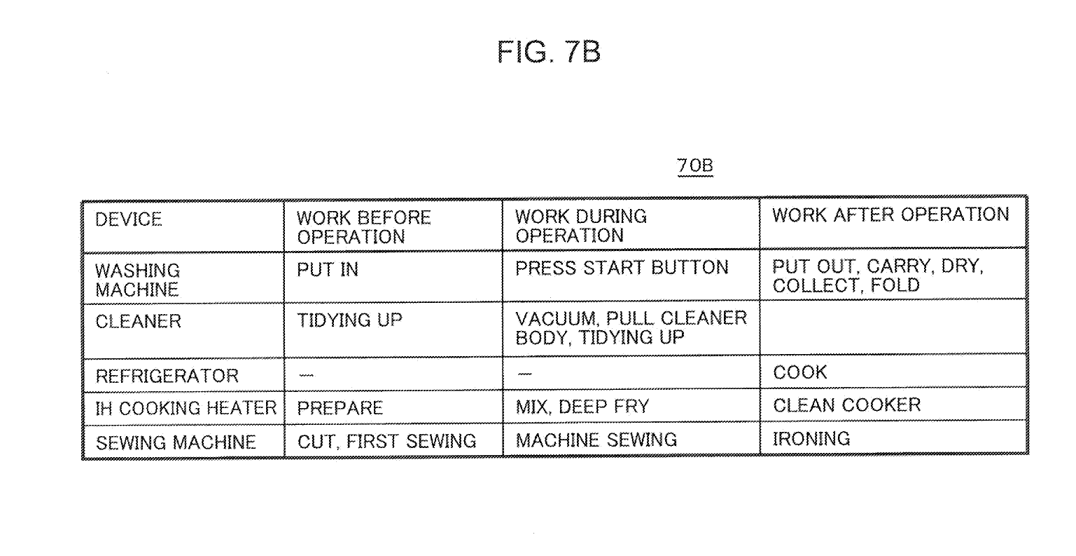

FIG. 7A is a diagram illustrating classification of log information data to be stored in the log information storage unit 612. The log information is classified into an operation log and a state log. The operation log indicates the contents of direct operations by the user with respect to the device 601. The state log indicates an internal state and an output state of the device 601. Examples of the output state include display information, sounds, or light notified to the user by the device 601.

<Operation Analysis>

FIG. 7B is a diagram illustrating an example of a table 70B, in which the user's work contents to be analyzed by the movement analysis unit 621 are mapped for each device 601. The table 70B is stored in e.g. the device information DB 624, and is used when the movement analysis unit 621 analyzes the user's movement with use of the log information. As illustrated in FIG. 7B, it is assumed that fatigue by the household chores is generated by three works i.e. a work before operation, a work during operation, and a work after operation with respect to each device 601.

In the example of FIG. 7B, assuming that the device is a washing machine, “PUT IN”, which is a work of putting the laundry in the washing machine, is defined as a work before operation. Further, “PRESS START BUTTON”, which is a work of pressing the start button of the washing machine, is defined as a work during operation. Further. “PUT OUT”, which is a work of putting the cleaned laundry out of the washing machine, “CARRY”, which is a work of carrying the laundry to a drying place. “COLLECT”, which is a work of collecting the dried laundry, and “FOLD”, which is a work of folding the collected laundry, are defined as a work after operation.

The movement analysis unit 621 analyzes the work before operation relating to washing by extracting the log information relating to the work “PUT IN” from the log information relating to washing by the user. Specifically, the movement analysis unit 621 analyzes the work before operation relating to washing by extracting the log information indicating a start timing and an end timing of the work “PUT IN” from the log information relating to washing by the user.

Further, the movement analysis unit 621 analyzes the work during operation relating to washing by extracting the log information indicating the work “PRESS START BUTTON” from the log information relating to washing by the user. Further, the movement analysis unit 621 analyzes the work after operation relating to washing by extracting the log information indicating a start timing and an end timing of the work from the log information relating to washing by the user with respect to each of the works “PUT OUT”, “CARRY”, and the like. The contents of log information to be extracted for each work are determined in advance. Accordingly, the movement analysis unit 621 may extract log information in accordance with the predetermined contents.

Likewise, as well as the washing machine, the movement analysis unit 621 may analyze the work before operation, the work during operation, and the work after operation defined in the table 70B for the devices 601 such as a cleaner and a refrigerator, other than the washing machine.

<Estimation of Fatigue Part>

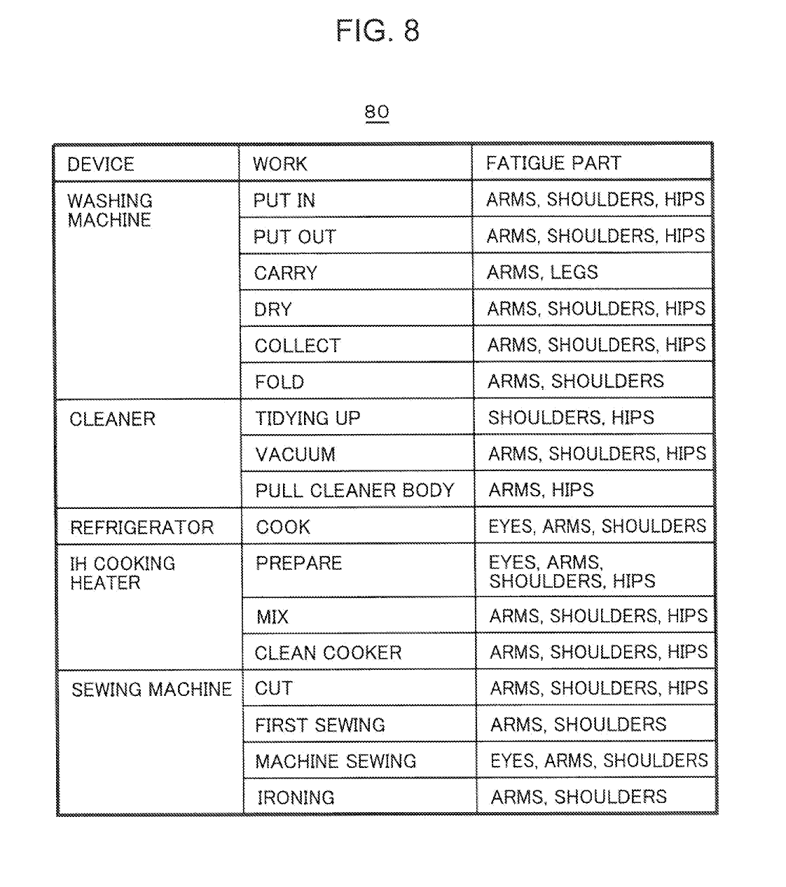

FIG. 8 is a diagram illustrating an example of a table 80, in which fatigue parts of the user to be estimated by the fatigue degree estimation unit 622 are mapped for each device. The table 80 is stored in the fatigue degree parameter DB 625, and is referred to when the fatigue degree estimation unit 622 estimates the fatigue degree.

As illustrated in FIG. 8, the fatigue parts resulting from doing the household chores are presumed for individual works which are presumed by the movement analysis unit 621. For instance, it is assumed that regarding a cleaner, the user feels the shoulders and the hips heavy in tidying up, which is a work before cleaning, feels the arms, the shoulders, and the hips heavy during cleaning, and feels the arms and the hips heavy each time the user pulls the cleaner body.

In view of the above, in the table 80. “SHOULDERS”, and “HIPS” are mapped to “TIDYING UP” of “CLEANER”. Thus, when the work of tidying up in cleaning is analyzed by the movement analysis unit 621, the fatigue degree estimation unit 622 estimates fatigue degrees of “SHOULDERS” and “HIPS” from the log information relating to the work. Regarding the devices 601 such as a washing machine and a refrigerator, other than the cleaner, as with the case of the cleaner, the fatigue degree estimation unit 622 estimates fatigue degrees of the fatigue parts with respect to the work in accordance with the table 80.

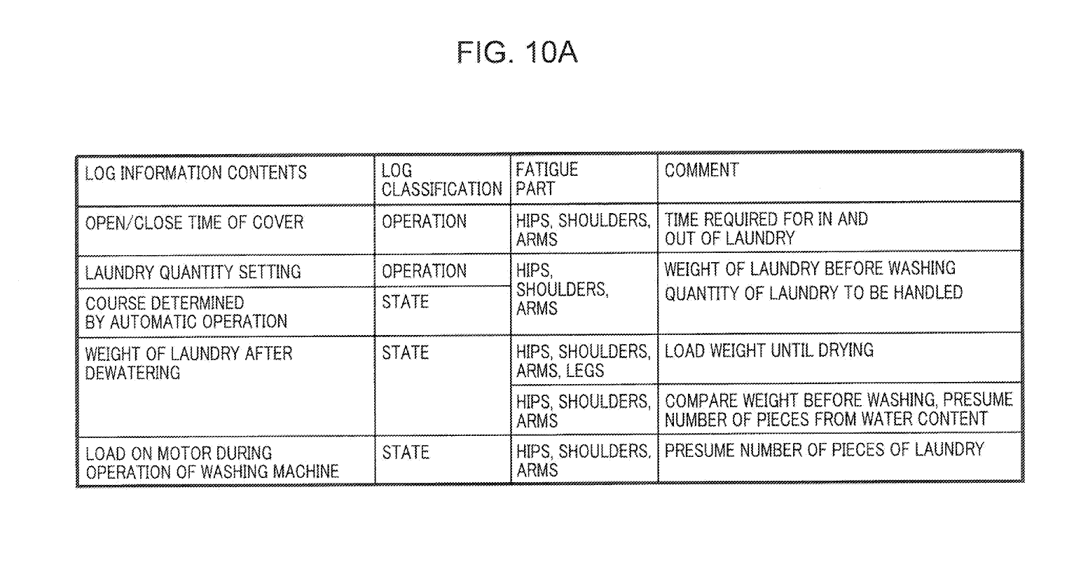

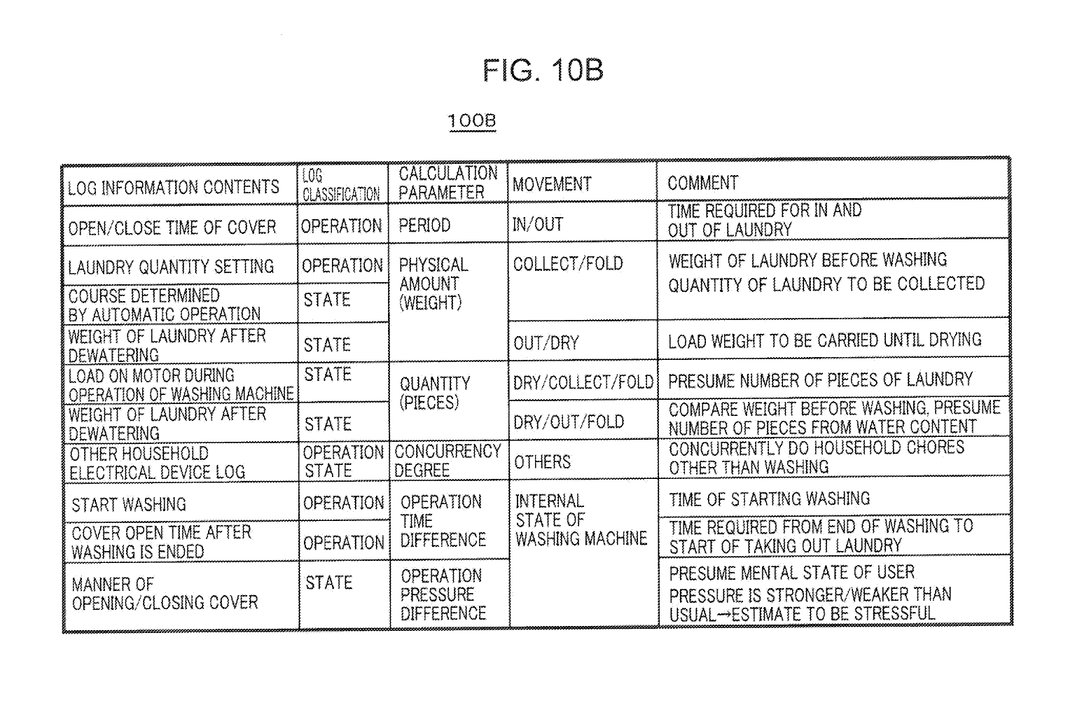

FIG. 10A is a diagram illustrating an example of fatigue parts to be estimated from washing machine log information. In FIG. 10A, “FATIGUE PARTS” as parts to be estimated with respect to “LOG INFORMATION CONTENTS” are defined. For instance, the movement analysis unit 621 extracts the log information relating to a point of time of opening/closing the cover of the washing machine, as log information indicating a working time of the work relating to “PUT IN” or “PUT OUT” of the laundry. The fatigue degree estimation unit 622 estimates fatigue degrees of “HIPS”, “SHOULDERS”, and “ARMS” from the working time. Further, the movement analysis unit 621 extracts the log information relating to laundry quantity setting, as log information relating to the work “PUT IN” or “PUT OUT” of the laundry, because the log information relating to laundry quantity setting is the log information relating to “WEIGHT OF LAUNDRY BEFORE WASHING” or “WEIGHT OF LAUNDRY TO BE COLLECTED”. The fatigue degree estimation unit 622 estimates fatigue degrees of “HIPS”, “SHOULDERS”, and “ARMS” from the log information.

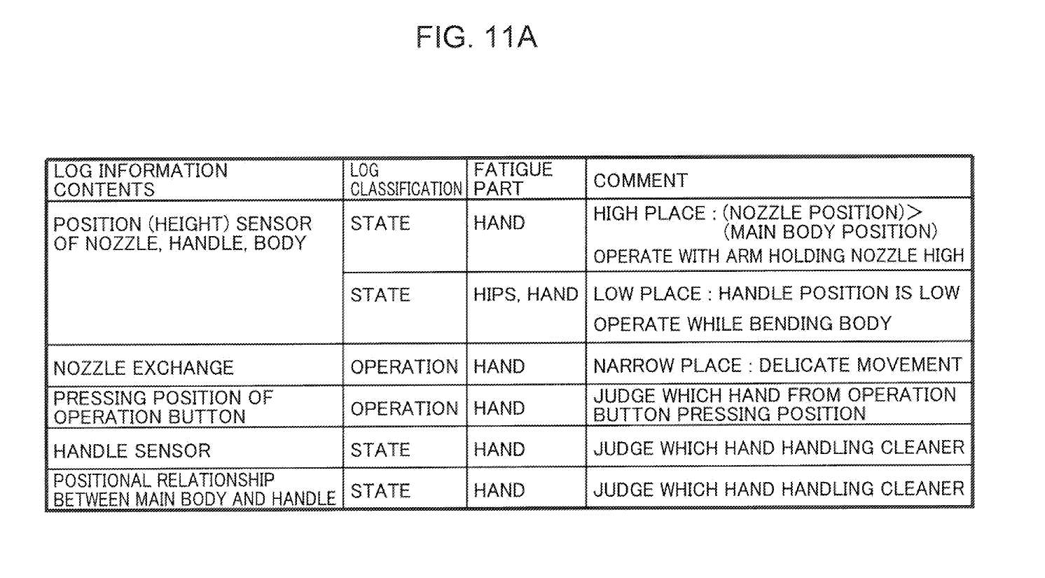

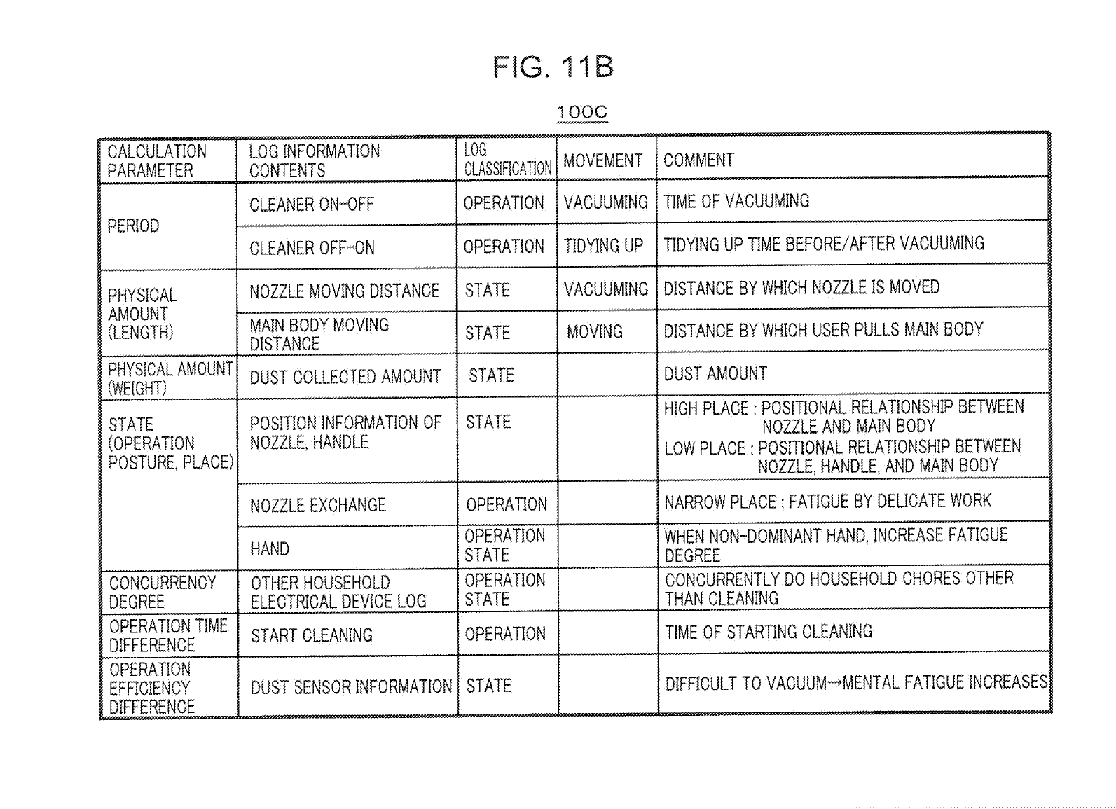

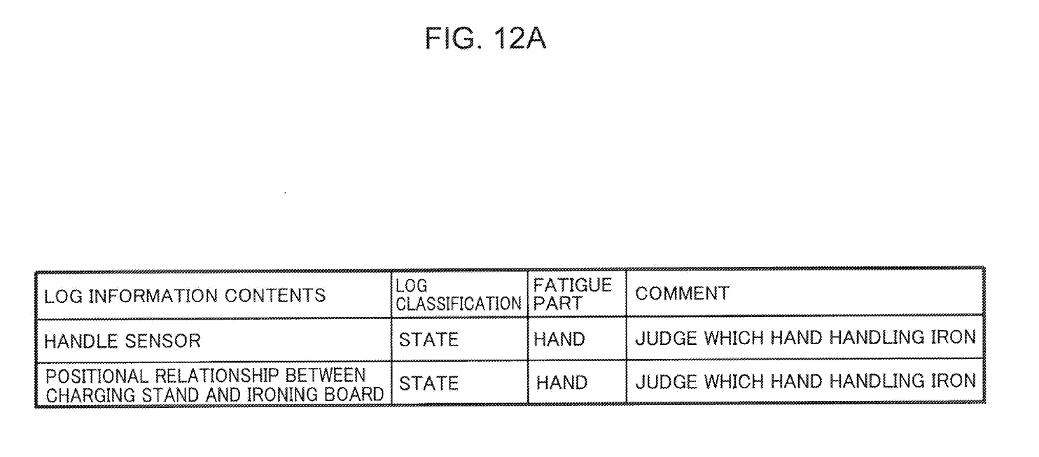

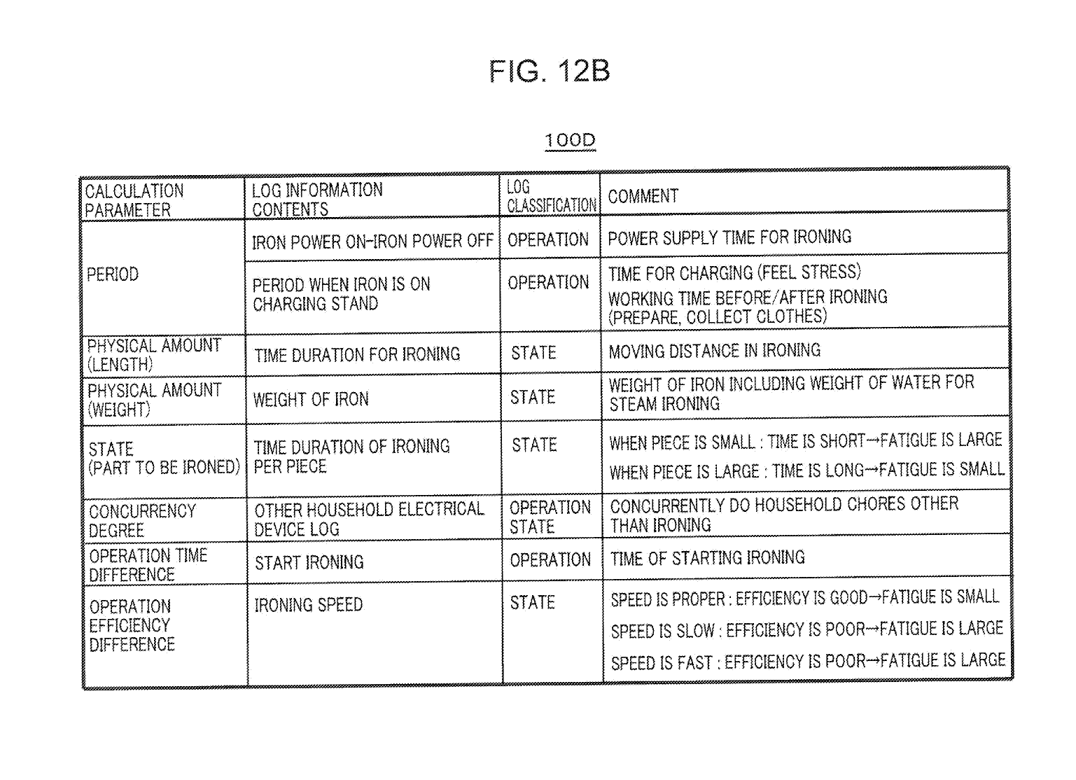

FIG. 11A is a diagram illustrating an example of fatigue parts to be estimated from cleaner log information. FIG. 12A is a diagram illustrating an example of fatigue parts to be estimated from iron log information. The details illustrated in FIG. 11A and in FIG. 12A are the same as those in FIG. 10A.

<Calculation Parameter>

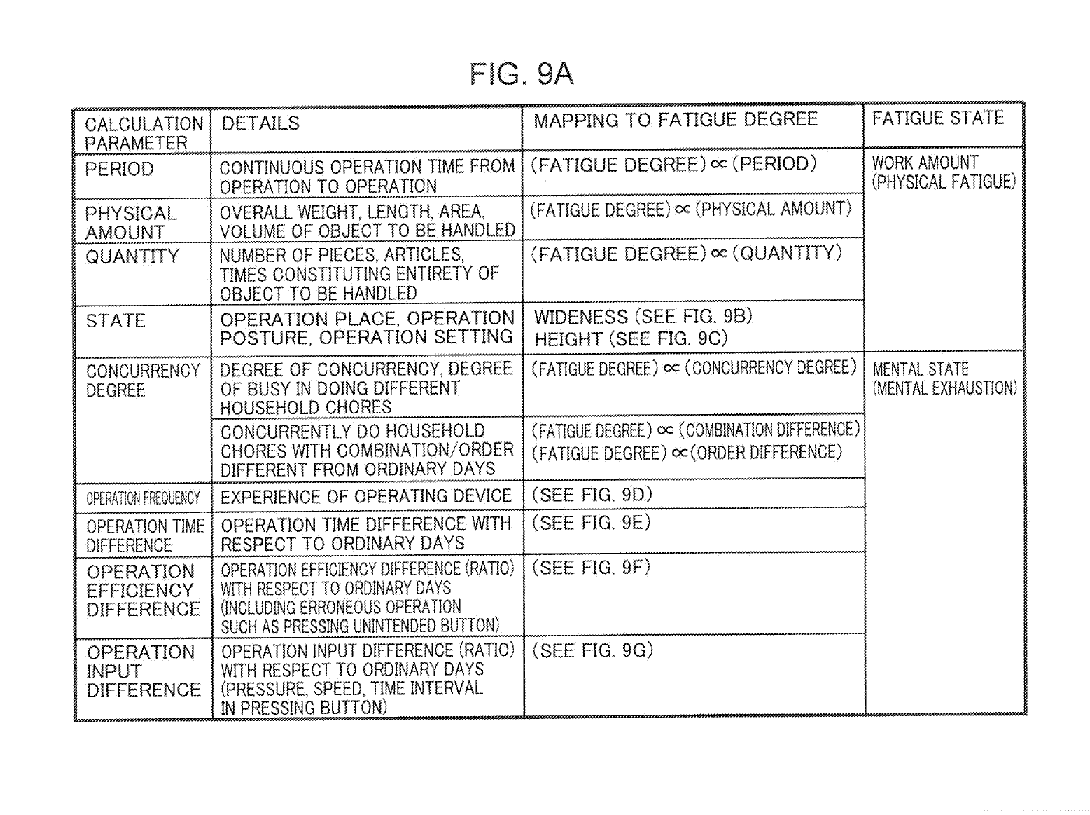

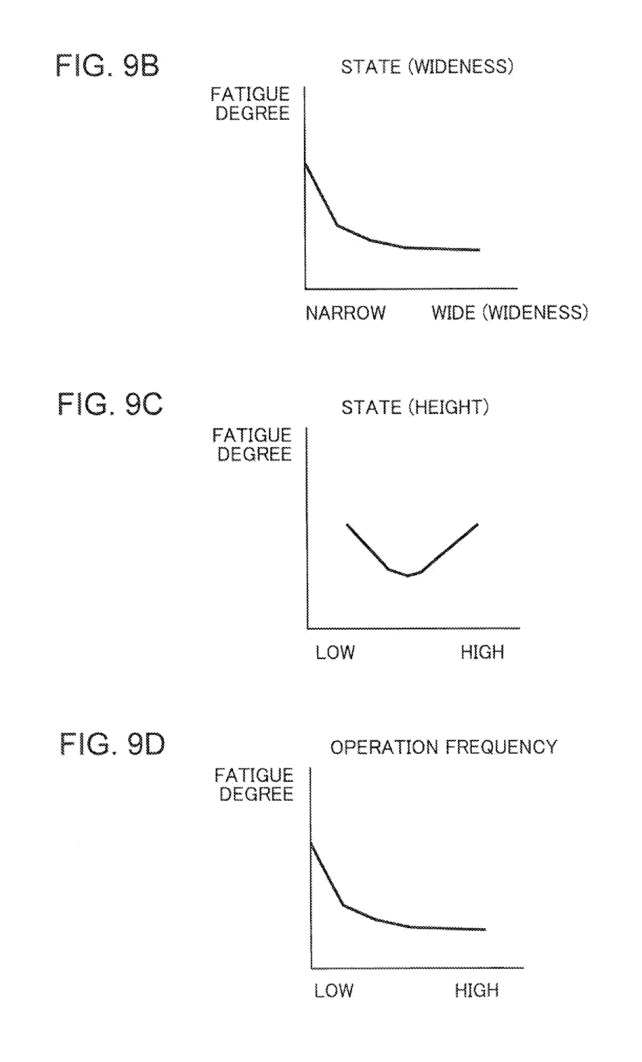

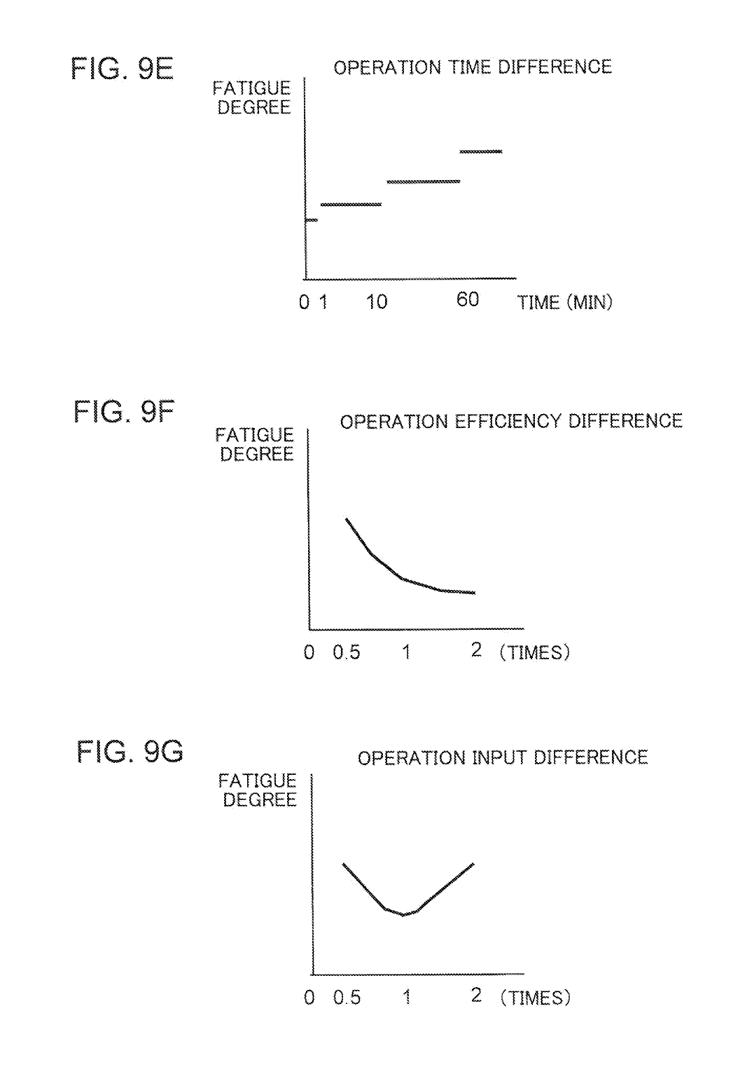

FIG. 9A is a diagram illustrating an example of a relationship between calculation parameters for use in calculating fatigue degrees by the fatigue degree estimation unit 622, and the fatigue degrees. As illustrated in FIG. 9A, as “CALCULATION PARAMETERS”, there are defined a time period, a physical amount, a quantity, a state, a degree of concurrency, a frequency of operations, an operation time difference, an operation efficiency difference, and an operation input difference. A relationship between each calculation parameter and a fatigue degree is defined by “MAPPING TO FATIGUE DEGREE”. Among the calculation parameters, the time period, the physical amount, the quantity, and the state are calculation parameters by which a physical fatigue degree resulting from doing an actual work is estimated, and the degree of concurrency, the frequency of operations, the operation time difference, the operation efficiency difference, and the operation input difference are calculation parameters by which a fatigue degree of a mental part accompanied by doing the household chores is estimated.

For instance, a calculation parameter “PERIOD” is a calculation parameter relating to a continuous operation time from a certain operation (e.g. an operation of opening the cover of the washing machine) to a certain operation (e.g. an operation of closing the cover of the washing machine). The calculation parameter “PERIOD” is defined in such a manner that the fatigue degree increases as “PERIOD” increases, as illustrated in “MAPPING TO FATIGUE DEGREE”.

Further, the calculation parameter “PHYSICAL AMOUNT” is a calculation parameter relating to the weight, the length, the area, and the volume of the entirety of an object to be handled (e.g. laundry). The calculation parameter “PHYSICAL AMOUNT” is defined in such a manner that the fatigue degree increases as the physical amount increases, as illustrated in “MAPPING TO FATIGUE DEGREE”.

Further, the calculation parameter “QUANTITY” is a calculation parameter relating to the number of pieces or the number of articles of the entirety of the object to be handled (e.g. laundry), or the number of times of doing the laundry. The calculation parameter “QUANTITY” is defined in such a manner that the fatigue degree increases, as the quantity increases.

Further, the calculation parameter “STATE” is a calculation parameter relating to the operation place, the operation posture, and the operation setting of the device 601. The calculation parameter “STATE” is classified into wideness and height. The calculation parameter “STATE (WIDENESS)” is illustrated in FIG. 9B. The calculation parameter “STATE (WIDENESS)” is defined in such a manner that the fatigue degree increases, as the state is narrowed. This is based on the idea that, assuming that the device is a cleaner, obstacles in cleaning are densely disposed in a narrow space, and when the narrow space is cleaned, a work such as avoiding the obstacles or moving the obstacles is generated, which may increase the fatigue degree.

The calculation parameter “STATE (HEIGHT)” is illustrated in FIG. 9C. The calculation parameter “STATE (HEIGHT)” is defined in such a manner that the fatigue degree increases, as the place for cleaning is a higher place or a lower place. This is based on the idea that, assuming that the device is a cleaner, when the user cleans a high place, the user is required to work while lifting the nozzle of the cleaner high above the user's head, which may increase a physical burden, and when the user cleans a low place, the user is required to work while stooping, which may also increase a physical burden.

The calculation parameter “CONCURRENCY DEGREE” is a calculation parameter, considering a case, in which the user does another household chore concurrently, or the user does multiple household chores concurrently with a combination or in the order different from the ordinary pattern. The calculation parameter “CONCURRENCY DEGREE” is defined in such a manner that the fatigue degree increases, as the degree of concurrency indicating the number of household chores to be done concurrently increases. Further, the calculation parameter “CONCURRENCY DEGREE” is defined in such a manner that the fatigue degree increases, as a difference between combination of household chores to be done on ordinary days and combination of household chores to be done on this day increases. Further, the calculation parameter “CONCURRENCY DEGREE” is defined in such a manner that the fatigue degree increases, as the difference between the order of household chores on ordinary days and the order of household chores on this day increases.

The calculation parameter “OPERATION FREQUENCY” is a calculation parameter, considering the skill of the user in operating the device 601. The calculation parameter “OPERATION FREQUENCY” is illustrated in FIG. 9D. The calculation parameter “OPERATION FREQUENCY” is defined in such a manner that the fatigue degree increases, as the frequency of operations is lowered. This is based on the idea that when the user is not accustomed to the operation of the device 601, mental burden of the user may increase.

The calculation parameter “OPERATION TIME DIFFERENCE” is a calculation parameter on the operation time of the device 601, considering a difference in operation time between ordinary days and this day. The calculation parameter “OPERATION TIME DIFFERENCE” is illustrated in FIG. 9E. The calculation parameter “OPERATION TIME DIFFERENCE” is defined in such a manner that the fatigue degree stepwise increases, as the operation time difference increases. This is based on the idea that as the operation time of the device 601 on this day increases as compared with an operation time on ordinary days, the user may feel exhausted, and mental burden of the user may increase.

The calculation parameter “OPERATION EFFICIENCY DIFFERENCE” is a calculation parameter on the operation efficiency of the device 601, considering a difference in operation efficiency between ordinary days and this day. The calculation parameter “OPERATION EFFICIENCY DIFFERENCE” is illustrated in FIG. 9F. The calculation parameter “OPERATION EFFICIENCY DIFFERENCE” is defined in such a manner that the fatigue degree increases, as the ratio of operation efficiency on this day with respect to operation efficiency on ordinary days is lowered. This is based on the idea that as the operation efficiency is lowered (e.g. erroneous input of an operation instruction), the user may feel exhausted, and mental burden of the user may increase.

The calculation parameter “OPERATION INPUT DIFFERENCE” is a calculation parameter on an operation input to the device 601, considering a difference in operation input between ordinary days and this day. The calculation parameter “OPERATION INPUT DIFFERENCE” is illustrated in FIG. 9G. The calculation parameter “OPERATION INPUT DIFFERENCE” is defined in such a manner that the fatigue degree increases, as the ratio of operation input on this day with respect to operation input on ordinary days is deviated from 1. This is based on the idea that as the operation input difference increases as compared with ordinary days, the user may feel exhausted, and mental burden of the user may increase. Examples of the operation input are a pressure of pressing the button of the device 601, the speed of pressing the button, and a time interval at which the button is pressed.

FIG. 10B is a diagram illustrating an example of a table 100B, in which washing machine log information and calculation parameters are mapped to each other. The table 10013 is stored in the fatigue degree parameter DB 625, and is used when the fatigue degree estimation unit 622 estimates the fatigue degree. The above idea is also applied to tables 100C and 100D to be described later.

In the table 100B, there are defined “CALCULATION PARAMETERS” to be derived from “LOG INFORMATION CONTENTS”. A fatigue degree is estimated for each of the contents defined in “MOVEMENT”. For instance, a working time of a work of putting the laundry in or out of the washing machine is calculated from the log information indicating the time of opening/closing the cover of the washing machine, and the calculation parameter “PERIOD” is set for the calculated working time, whereby a fatigue degree is estimated. Further, a weight of the laundry before washing is calculated from the log information “LAUNDRY QUANTITY SETTING”, and the calculation parameter “PHYSICAL AMOUNT” is set for the weight, whereby the fatigue degree is estimated. In this way, the fatigue degree estimation unit 622 sets the calculation parameters on the washing machine in accordance with the table 100B, and a fatigue degree is estimated.

FIG. 11B is a diagram illustrating an example of the table 100C, in which cleaner log information and calculation parameters are mapped to each other. FIG. 12B is a diagram illustrating an example of the table 100D, in which iron log information and calculation parameters are mapped to each other. The configuration of the tables 100C and 100D is the same as the configuration of the table 100B. In this way, the fatigue degree estimation unit 622 sets the calculation parameters on the cleaner in accordance with the table 100C, and estimates the fatigue degree. Likewise, the fatigue degree estimation unit 622 sets the calculation parameters on the iron in accordance with the table 100D, and estimates the fatigue degree.

The calculation parameters on a fatigue degree to be derived from the log information are as follows.

Refrigerator:

The calculation parameter “QUANTITY” is set from the number of times of taking out the grocery and ice cubes, and the calculation parameter “PHYSICAL AMOUNT” is set from the amount of the grocery and ice cubes that have been taken out.

IH cooking heater, microwave oven:

The calculation parameter “STATE” is set from the setting contents on a cooking menu, and the calculation parameter “PERIOD” is set from a cooking time.

Range hood fan:

The calculation parameter “PERIOD” is set from a driving time.

Dish washer:

The calculation parameter “PERIOD” is set from a driving time, and the calculation parameter “QUANTITY” is set from the number of tableware.

TV receiver:

The calculation parameter “PERIOD” is set from a viewing time and a 3D viewing time.

BD/DVD recorder:

The calculation parameter “STATE” is set from the contents of moving image editing, and the calculation parameter “PERIOD” is set from an editing time.

Air conditioner:

The calculation parameter “QUANTITY” is set from the number of times of cleaning the filter.

Active meter, smartphone, smart media:

The calculation parameter “PHYSICAL AMOUNT” is set from a moving distance, the calculation parameter “STATE is set from a route, and the calculation parameter “PERIOD” is set from a time.

<Estimation of Fatigue Degree>

A fatigue degree of a specific part resulting from operating a certain device 601 is calculated, as expressed by the following formula (1), using a calculation parameter, a past average value of the calculation parameter, and a weighting coefficient of the calculation parameter.

$$\begin{matrix} [ Formula 1 ] & \\ ( fatigue degree of part j with respect to device i ) = {\sum }_{k} \{ ( P\_ijk ) / ( Pave\_ijk ) * ( W\_ijk ) \} & ( 1 )\end{matrix}$$

P_ijk: a value of the calculation parameter k of the part j with respect to the device i

Pave_ij: a past average value of the calculation parameter k of the part j with respect to the device i

W_ijk: a weighting coefficient of the calculation parameter k of the part j with respect to the device i

As expressed by the following formula (2), a fatigue degree of each part is calculated by the sum of fatigue degrees of the target part resulting from operating each device 601.

$$\begin{matrix} [ Formula 2 ] & \\ ( fatigue degree of part j ) = {\sum }_{i} ( fatigue degree of part j with respect to device i ) & ( 2 )\end{matrix}$$

In the formula (1), the weighting coefficient W_ijk of the calculation parameter k of the part j is a fixed value that has been determined in advance by evaluation. Further, in the formula (1) in the first embodiment, the fatigue degree of a specific part resulting from operating the device 601 is calculated as the sum of fatigue degrees relating to each of the calculation parameters k. Alternatively, the fatigue degree of a specific part may be calculated as an accumulation relating to each of the calculation parameter k, as expressed by the following formula (3).

$$\begin{matrix} [ Formula 3 ] & \\ ( fatigue degree of part j with respect to device i ) = {\prod }_{k} \{ 1 + ( P\_ijk - Pave\_ijk ) / ( Pave\_ijk ) * ( W\_ijk ) \} & ( 3 )\end{matrix}$$

P_ijk: a value of the calculation parameter k of the part j with respect to the device i

Pave_ijk: a past average value of the calculation parameter k of the part j with respect to the device i

W_ijk: a weighting coefficient of the calculation parameter k of the part j with respect to the device i

<Description on Movement>

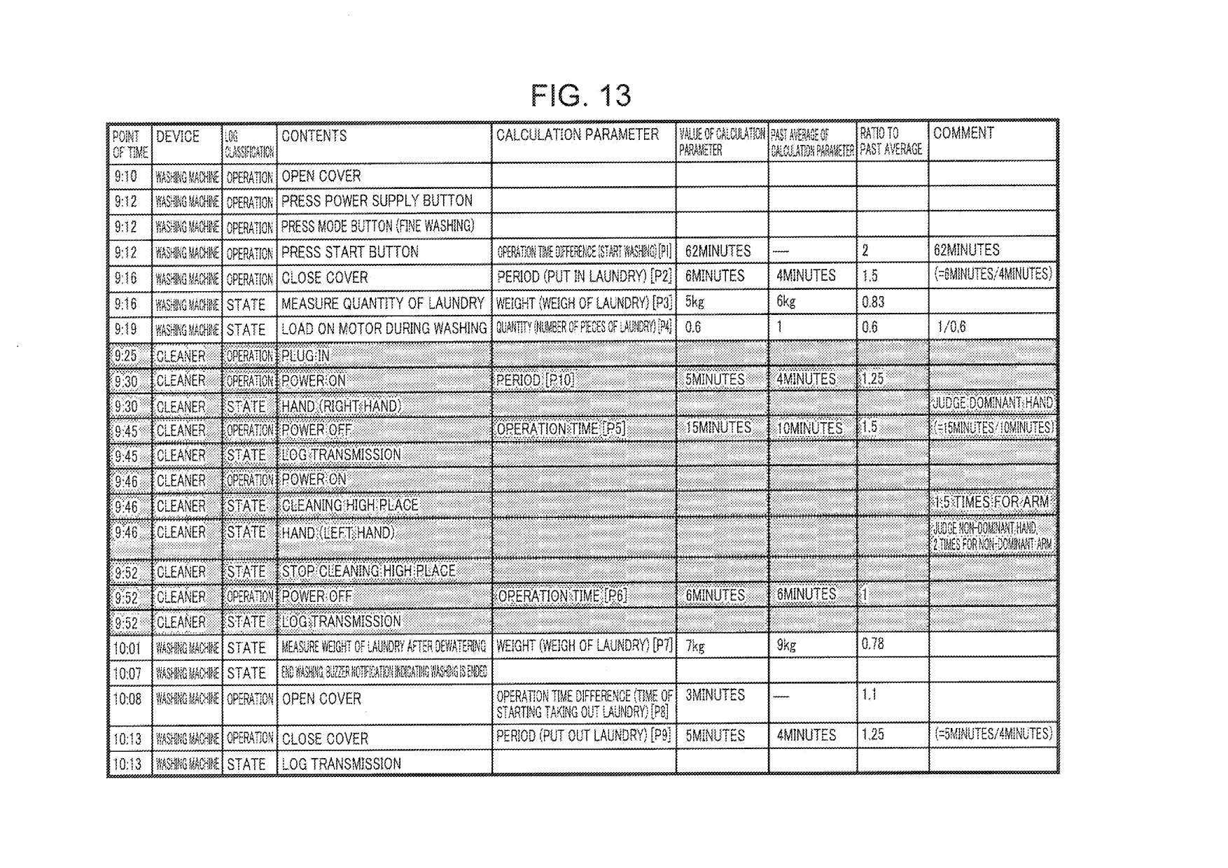

FIG. 13 is a diagram illustrating an example of user's log information stored in the log information DB 623. The log information in FIG. 13 is log information relating to washing and cleaning. In the following, fatigue degree estimation processing is described with use of the log information in FIG. 13. In the example of FIG. 13, first of all, washing with use of a washing machine is started at a.m. 9:10. During a time period from a.m. 9:25 to a.m. 9:52 in the course of washing, cleaning with use of a cleaner is performed concurrently with the washing. During a time period from a.m. 10:08 to a.m. 10:13, the laundry is taken out. The calculation parameters to be derived from the log information in FIG. 13 are as follows in the order of time of acquiring the data.

<a.m. 9:12> Operation Time Difference (P1) when Washing is Started

The movement analysis unit 621 extracts log information “PRESS START BUTTON” as log information relating to the work “START WASHING”. Then, the fatigue degree estimation unit 622 sets the difference between the time when the start button has been pressed on this day and the time when the start button is pressed on ordinary days, as a calculation parameter P1 “OPERATION TIME DIFFERENCE”.

<a.m. 9:16> Working Time (P2) when Laundry is Put in Washing Machine

The movement analysis unit 621 extracts log information indicating “OPEN COVER” of the washing machine, and log information indicating “CLOSE COVER”, as log information relating to the work “PUT IN LAUNDRY” (corresponding to the work before operation). Then, the fatigue degree estimation unit 622 sets the time difference between the extracted log information as a working time of “PUT IN LAUNDRY”, and sets the working time as a calculation parameter P2 “PERIOD”.

<a.m. 9:19> Weight (P3) of Laundry Before Washing

The movement analysis unit 621 extracts log information “MEASURE QUANTITY OF LAUNDRY” as log information relating to the work “PUT IN LAUNDRY”. Then, the fatigue degree estimation unit 622 sets “WEIGHT (WEIGHT OF LAUNDRY)” as indicated by the log information as a calculation parameter P3 “PHYSICAL AMOUNT”.

<a.m. 9:19> Number (P4) of Pieces of Laundry

The movement analysis unit 621 extracts log information “LOAD ON MOTOR DURING WASHING” as log information relating to the work “PUT IN LAUNDRY”. Then, the fatigue degree estimation unit 622 sets “QUANTITY (NUMBER OF PIECES OF LAUNDRY) as indicated by the log information as a calculation parameter P4 “PHYSICAL AMOUNT”.

<a.m. 9:30> Tidying Up Time (P10)

The movement analysis unit 621 extracts log information “PLUG IN” and log information “POWER ON” of the cleaner, as log information relating to the work “TIDYING UP” relating to cleaning by the cleaner (corresponding to the work before operation). Then, the fatigue degree estimation unit 622 sets the time difference between the log information as a working time of “TIDYING UP”, and sets the working time as a calculation parameter P10 “PERIOD”.

<a.m. 9:45> Operation Time (P5) of Cleaner

The movement analysis unit 621 extracts log information “POWER ON” and log information “POWER OFF” of the cleaner as log information relating to the operation of the cleaner. Then, the fatigue degree estimation unit 622 sets the time difference between the log information as a calculation parameter P5 “PERIOD”.

<a.m. 9:52> Operation Time (P6) of Cleaner in High Place by Non-Dominant Hand

The movement analysis unit 621 extracts log information “POWER ON” indicating that the power of the cleaner is turned on, and log information “POWER OFF”, as log information relating to the operation of the cleaner. Then, the fatigue degree estimation unit 622 sets the time difference between the log information as a calculation parameter P6 “PERIOD”.

In the above case, the fatigue degree estimation unit 622 increases the fatigue degree, because the state of the cleaner indicates “CLEANING HIGH PLACE”, and the user operated the cleaner with the suction port of the cleaner being directed toward a high place. It is possible to determine that the cleaner cleans a high place by e.g. providing height sensors HS1 and HS2 at a distal end and the middle of the hose of the cleaner, and providing a height sensor HS3 on the cleaner main body, and if the measurement value difference between the height sensors HS1 and HS3 is not smaller than a predetermined value. On the other hand, it is possible to determine that the cleaner cleans a low place if the measurement value difference between the height sensors HS1 and HS2 is not larger than a predetermined value.

<a.m. 10:01> Weight (P7) of Laundry after Dewatering

The movement analysis unit 621 extracts log information “MEASURE WEIGHT OF LAUNDRY AFTER DEWATERING” regarding the washing machine, as log information relating to the work “PUT OUT LAUNDRY” (corresponding to the work after operation). Then, the fatigue degree estimation unit 622 sets “WEIGHT (WEIGHT OF LAUNDRY)” as indicated by the log information as a calculation parameter P7 “PHYSICAL AMOUNT”.

<a.m. 10:08> Period (P8) from Time when Buzzer Notifies that Washing is Ended to Time when Laundry is Started to be Taken Out

The movement analysis unit 621 extracts log information “OPERATION ENDED, BUZZER NOTIFICATION INDICATING OPERATION IS ENDED” and log information “OPEN COVER”, as log information on a time period from end of washing to start of taking out the laundry. Then, the fatigue degree estimation unit 622 sets the time difference between this day and ordinary days regarding a time period from end of washing to start of taking out the laundry, as a calculation parameter P8 “OPERATION TIME DIFFERENCE”.

<a.m. 10:13> Working Time (P9) of Taking Out Laundry

The movement analysis unit 621 extracts log information “OPEN COVER” and log information “CLOSE COVER” of the washing machine, as log information relating to the work “PUT OUT LAUNDRY” (corresponding to the work after operation). Then, the fatigue degree estimation unit 622 sets the time difference between the extracted log information as a working time of “PUT OUT LAUNDRY”, and sets the working time as a calculation parameter P9 “PERIOD”.

In FIG. 13, “PAST AVERAGE OF CALCULATION PARAMETER” is calculated from the past log information stored in the log information DB 623. Further, in FIG. 13, the “RATIO TO PAST AVERAGE” is a ratio of “CALCULATION PARAMETER” on this day with respect to “PAST AVERAGE OF CALCULATION PARAMETER”.

<Calculation Method of Ratio of P1 to Past Average>

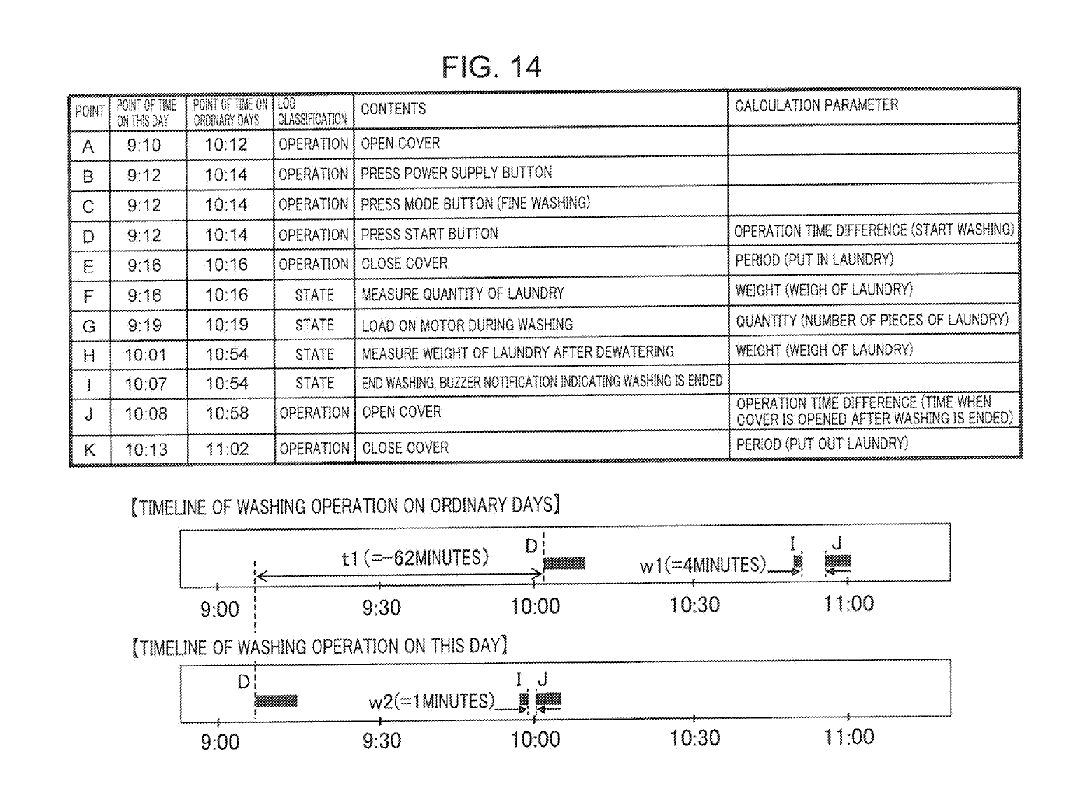

FIG. 14 is a diagram illustrating a comparison between this day and ordinary days with respect to washing machine log information. An example of a time on ordinary days is a representative value of a time when the same work has been done in the past, such as an average value of a working time in the past, or a time that appears most frequently. In FIG. 14, the upper portion indicates a time sequence of a washing operation on ordinary days, and the lower portion indicates a time sequence of a washing operation on this day.

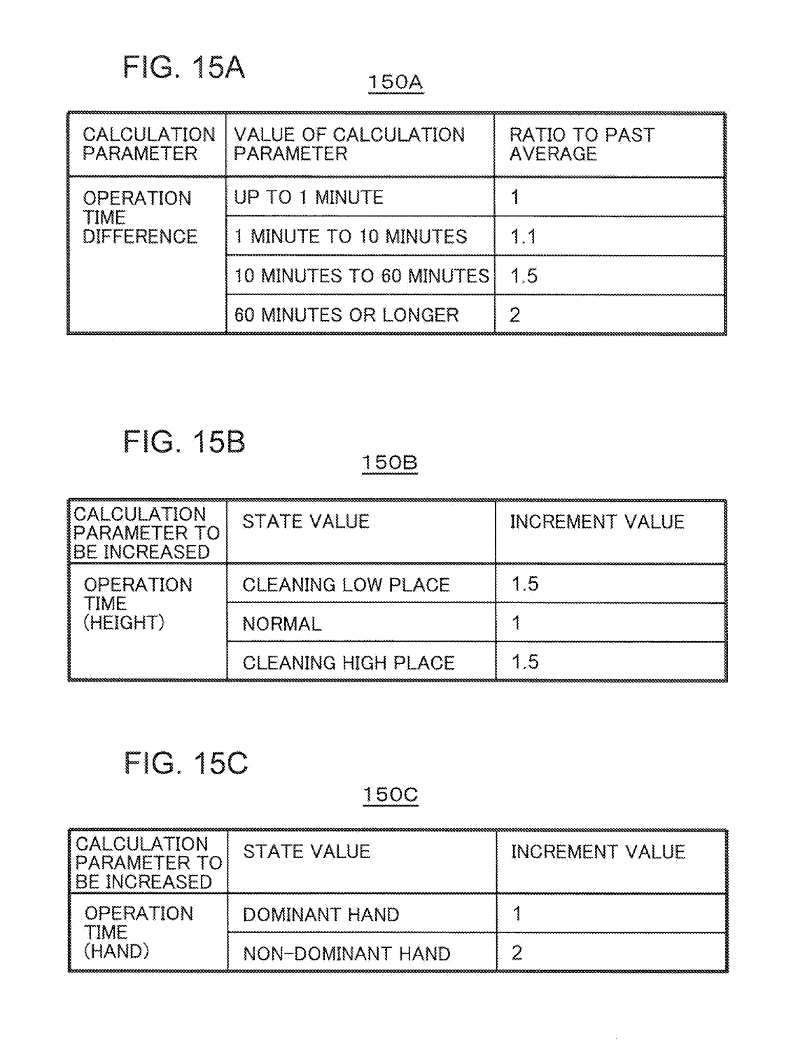

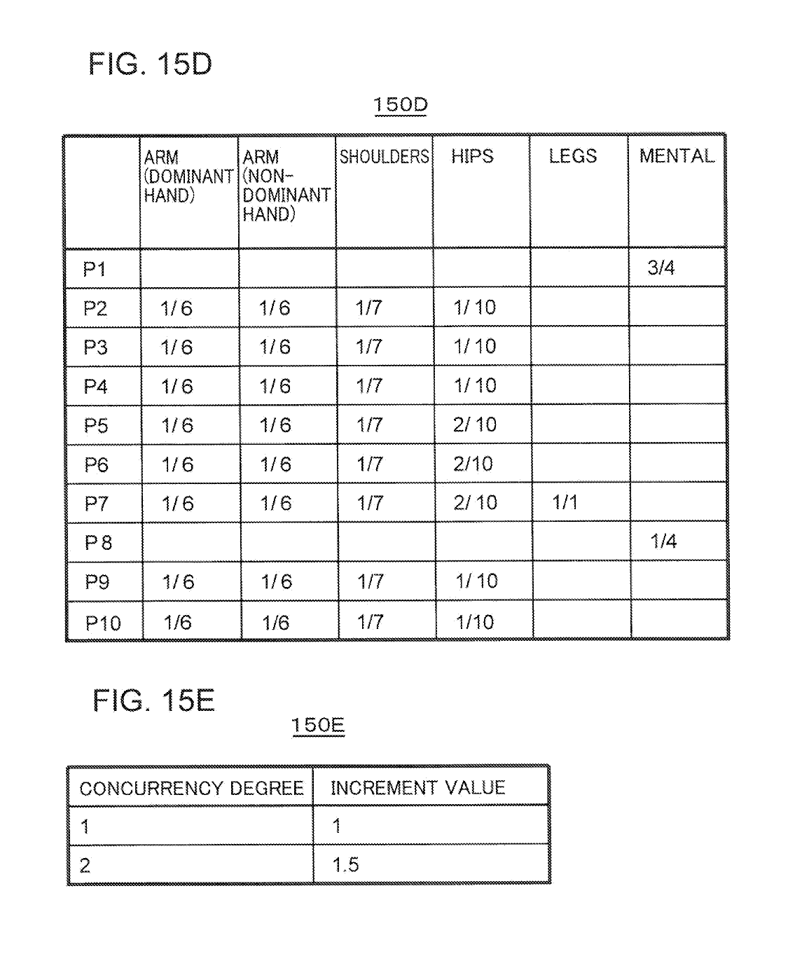

FIG. 15A is a diagram illustrating an example of a table 150A to be used in calculating “RATIO TO PAST AVERAGE” regarding the calculation parameter (P1, P8) “OPERATION TIME DIFFERENCE”. The table 150A defines “CALCULATION PARAMETER”, “VALUE OF CALCULATION PARAMETER”, and “RATIO TO PAST AVERAGE”. “CALCULATION PARAMETER” defines “OPERATION TIME DIFFERENCE” indicating the type of the calculation parameter. “VALUE OF CALCULATION PARAMETER” defines a class to which the value of the calculation parameter belongs. In this example, the value of the calculation parameter is classified into four classes “UP TO 1 MINUTE” indicating one minute or shorter, “1 MINUTE TO 10 MINUTES” indicating not shorter than one minute but shorter than ten minutes, “10 MINUTES TO 60 MINUTES” indicating not shorter than 10 minutes but shorter than 60 minutes, and “60 MINUTES OR LONGER” indicating 60 minutes or longer.

“RATIO TO PAST AVERAGE” defines “RATIO TO PAST AVERAGE” in each class of the value of the calculation parameter.

In the example of FIG. 15A, “RATIO TO PAST AVERAGE” defines the values that stepwise increases in the range of from 1 to 2, such as “1”, “1.1”, “1.5”, and “2”. This is based on the idea that as the operation time difference between ordinary days and this day increases, mental burden of the user may increase.

In the example of FIG. 14, the calculation parameter P1 “OPERATION TIME DIFFERENCE” at the time of starting washing is calculated as follows. The point D indicates a timing at which the start button is pressed. As illustrated in the upper portion of FIG. 14, the time of the point D on ordinary days is 10:14, and as illustrated in the lower portion of FIG. 14, the time of the point D on this day is 9:12. Therefore, the calculation parameter P1 is sixty-two minutes, which is the absolute value of a time difference t1 between ordinary days and this day at the point D. The sixty-two minutes belongs to the class “60 MINUTES OR LONGER” in FIG. 15. Accordingly, the fatigue degree estimation unit 622 calculates that “RATIO TO PAST AVERAGE” is “2”.

<Calculation Method of Ratio of P2 to Past Average>

The calculation method is described referring to FIG. 14. The calculation parameter P2 relating to the working time of “PUT IN LAUNDRY” is a time period from the point A when the cover of the washing machine is opened to the point E when the cover is closed. On this day, the time of the point A is 9:10, and the time of the point E is 9:16. Accordingly, a time period from the point A to the point E on this day is six minutes. Likewise, a time period from the point A (time=10:12) to the point E (time=10:16) on ordinary days is four minutes. Accordingly, the fatigue degree estimation unit 622 calculates that the ratio of the calculation parameter P2 to past average is 1.5 (=6 minutes/4 minutes).

<Calculation Method of Ratio P3 to Past Average>

In FIG. 13, the calculation parameter P3 indicating the weight of laundry at the time of starting washing is such that the value is “5 kg” and the past average is “6 kg”. Accordingly, the fatigue degree estimation unit 622 calculates that the ratio of the calculation parameter P3 to past average is 0.83 (=5 kg/6 kg).

<Calculation Method of Ratio of P4 to Past Average>

The calculation parameter P4 indicating the number of pieces of laundry is determined by extracting a load state on the motor during a washing operation from the log information. In FIG. 13, the calculation parameter P4 is such that the value is “0.6” and the past average is “1”. Accordingly, the fatigue degree estimation unit 622 calculates the ratio of the calculation parameter P4 to past average is 0.6 (=0.6/1).

<Calculation Method of Ratio of P5 to Past Average>

In FIG. 13, the value of the calculation parameter P5 indicating the operation time of the cleaner is a time period from the time (time=9:30) when the power of the cleaner is turned on to the time (time=9:45) when the power of the cleaner is turned off, which is “15 MINUTES”. On the other hand, the past average of the calculation parameter is “10 MINUTES”. Accordingly, the fatigue degree estimation unit 622 calculates that the ratio of the calculation parameter P5 to past average is 1.5 (=15 minutes/10 minutes).

<Calculation Method of Ratio of P6 to Past Average>

In FIG. 13, the value of the calculation parameter P6 relating to the operation time of the cleaner in a high place is a time period from the time (time=9:46) when the power of the cleaner is turned on to the time (time=9:52) when the power of the cleaner is turned off, which is 6 minutes. On the other hand, the past average of the calculation parameter is also 6 minutes. Accordingly, the fatigue degree estimation unit 622 calculates that the ratio of the calculation parameter P6 to past average is 1 (=6 minutes/6 minutes).

The state of the cleaner log information is “CLEANING HIGH PLACE”. This shows that the place to be cleaned by the cleaner is a high place. In view of the above, the fatigue degree estimation unit 622 increases the fatigue degree with respect to cleaning a high place. FIG. 15B is a diagram illustrating an example of a table 150B to be used in determining an increment for the fatigue degree in cleaning a high place and in cleaning a low place. The table 150B defines “CALCULATION PARAMETER TO BE INCREASED”, “STATE VALUE”, and “INCREMENT VALUE”. “CALCULATION PARAMETER TO BE INCREASED” defines “OPERATION TIME” indicating the type of the calculation parameter to be increased, and “HEIGHT”, which is a factor of increase. “STATE VALUE” defines a state value of log information relating to an operation of the cleaner. In this example, “CLEANING LOW PLACE”, “NORMAL”, and “CLEANING HIGH PLACE” are defined. “INCREMENT VALUE” defines an increment value for a state value. In the example of the table 150B, the fatigue decree by cleaning a high place and cleaning a low place is 1.5 times of the fatigue degree by cleaning a place of a normal height.

Further, handling hand information in the cleaner log information indicates that the user works with the non-dominant hand, which is less used on ordinary days. In view of the above, the fatigue degree estimation unit 622 increases a fatigue degree with respect to the handling hand (arm). FIG. 15C is a diagram illustrating an example of a table 150C for use in determining an increment for the fatigue degree relating to the operation state (handling hand) of the cleaner. The table 150C defines “CALCULATION PARAMETER TO BE INCREASED”, “STATE VALUE”, and “INCREMENT VALUE”. “CALCULATION PARAMETER TO BE INCREASED” defines “OPERATION TIME” indicating the type of the calculation parameter to be increased, and “HANDLING HAND”, which is a factor of increase. “STATE VALUE” defines a state value of log information relating to the operation of the cleaner. In this example, “STATE VALUE” defines “DOMINANT HAND” indicating that the cleaner is operated by the dominant hand of the user, and “NON-DOMINANT HAND” indicating that the cleaner is operated by the non-dominant hand of the user. “INCREMENT VALUE” defines an increment value for a state value. In the example of the table 150C, the fatigue degree of work by the non-dominant hand is set to be two times of the fatigue degree of work by the dominant hand. It is possible to judge the dominant hand of the user by providing a plurality of contact sensors at positions of the cleaner to be held by the user in cleaning, and based on a detection pattern of the contact sensors.

<Calculation Method of Ratio of P7 to Past Average>

In FIG. 13, the calculation parameter P7 indicating the weight of laundry after dewatering is such that the value is 7 kg, and the past average is 9 kg. Accordingly, the fatigue degree estimation unit 622 calculates that the ratio of the calculation parameter P7 to average past is 0.78 (=7 kg/9 kg).

<Calculation Method of Ratio of P8 to Past Average>

The calculation parameter P8 indicating a time period from the time when the buzzer notifies that washing is ended to the time when the laundry is started to be taken out is calculated as follows. As illustrated in FIG. 14, a time period from the point I (time=10:07) to the point J (time=10:08) on this day is one minute. On the other hand, a time period from the point I (time=10:54) to the point J (time=10:58) on ordinary days is four minutes. Accordingly, the value of the calculation parameter P8 is three minutes (=4−1).

In FIG. 15A, “3 MINUTES” belongs to the class “1 MINUTE TO 10 MINUTES”. Accordingly, the fatigue degree estimation unit 622 calculates that the ratio of the calculation parameter P8 to past average is “1.1”.

<Calculation Method of Ratio of P9 to Past Average>

The calculation parameter P9 indicating a working time required for taking out the laundry is a time period from the time when the cover of the washing machine is opened after washing is ended to the time when the cover is closed. In the example of FIG. 14, a time period from the point J (time=10:08) to the point K (time=10:13) on this day is five minutes. On the other hand, a time period from the point J (time=10:58) to the point K (time=1:02) on ordinary days is four minutes. Accordingly, the fatigue degree estimation unit 622 calculates that the ratio of the calculation parameter P9 to past average is 1.25 (=5 minutes/4 minutes).

<Calculation Method of Ratio P10 to Past Average>

The calculation parameter P10 indicating a working time required for tidying up before the user operates the cleaner is a time period from the time when the user plugs in the cleaner to the time when the power of the cleaner is turned on. In FIG. 13, the working time is five minutes. On the other hand, the past average of the working time is four minutes. Accordingly, the fatigue degree estimation unit 622 calculates that the ratio of the calculation parameter P10 to past average is 1.25 (=5/4).

<Calculation of Fatigue Degree for Each Part>

FIG. 15D is a diagram illustrating an example of a table 150D which defines a weighting coefficient of each of the calculation parameters for each part. The table 150D defines weighting coefficients to be applied to “ARM (DOMINANT HAND)”, “ARM (NON-DOMINANT HAND)”, “SHOULDERS”, “HIPS”, “LEGS”, and “MENTAL” regarding the calculation parameters P1 to P10. In the table 150D, the cells in which a weighting coefficient is not described are indicated as blank boxes.

The fatigue degree estimation unit 622 applies weighting to each of the calculation parameters in accordance with a weighting coefficient described in the table 150D, and calculates a fatigue degree for each part. The following is a calculation example of fatigue degrees in the log information illustrated in FIG. 13.

$$( fatigue degree of arm ( dominant arm ) ) = ( ratio of P 2 to past average ) * ( weighting coefficient of P 2 ) + ( ratio of P 3 to past average ) * ( weighting coefficient of P 3 ) + ( ratio of P 4 to past average ) * ( weighting coefficient of P 4 ) + ( ratio of P 5 to past average ) * ( weighting coefficient of P 5 ) + ( ratio of P 6 to past average ) * ( weighting coefficient of P 6 ) + ( ratio of P 7 to past average ) * ( weighting coefficient of P 7 ) + ( ratio of P 9 to past average ) * ( weighting coefficient of P 9 ) + ( ratio of P 10 to past average ) * ( weighting coefficient of P 10 ) = ( 1.5 ) * ( 1 / 6 ) + ( 0.83 ) * ( 1 / 6 ) + ( 0.6 ) * ( 1 / 6 ) + ( 1.5 ) * ( 1 / 6 ) + ( 1 * 1 ) * ( 1 / 6 ) + ( 0.78 ) * ( 1 / 6 ) + ( 1.25 ) * ( 1 / 6 ) + ( 1.25 ) * ( 1 / 6 ) = 1.45$$

In this example, I is multiplied by 1 regarding the calculation parameter P6, because the user did not operate the cleaner with his/her dominant hand.

$$( fatigue degree of arm ( non \text{-} dominant arm ) ) = ( ratio of P 2 to past average ) * ( weighting coefficient of P 2 ) + ( ratio of P 3 to past average ) * ( weighting coefficient of P 3 ) + ( ratio of P 4 to past average ) * ( weighting coefficient of P 4 ) + ( ratio of P 5 to past average ) * ( weighting coefficient of P 5 ) + ( ratio of P 6 to past average ) * ( weighting coefficient of P 6 ) + ( ratio of P 7 to past average ) * ( weighting coefficient of P 7 ) + ( ratio of P 9 to past average ) * ( weighting coefficient of P 9 ) + ( ratio of P 10 to past average ) * ( weighting coefficient of P 10 ) = ( 1.5 ) * ( 1 / 6 ) + ( 0.83 ) * ( 1 / 6 ) + ( 0.6 ) * ( 1 / 6 ) + ( 1.5 * 0 ) * ( 1 / 6 ) + ( 1 * 1.5 * 2 ) * ( 1 / 6 ) + ( 0.78 ) * ( 1 / 6 ) + ( 1.25 ) * ( 1 / 6 ) + ( 1.25 ) * ( 1 / 6 ) = 1.53$$

In this example, I is multiplied by 1.5 and by 2 regarding the calculation parameter P6, because the user cleaned a high place with his/her non-dominant arm.

$$( fatigue degree of shoulders ) = ( ratio of P 2 to past average ) * ( weighting coefficient of P 2 ) + ( ratio of P 3 to past average ) * ( weighting coefficient of P 3 ) + ( ratio of P 4 to past average ) * ( weighting coefficient of P 4 ) + ( ratio of P 5 to past average ) * ( weighting coefficient of P 5 ) + ( ratio of P 6 to past average ) * ( weighting coefficient of P 6 ) + ( ratio of P 7 to past average ) * ( weighting coefficient of P 7 ) + ( ratio of P 9 to past average ) * ( weighting coefficient of P 9 ) + ( ratio of P 10 to past average ) * ( weighting coefficient of P 10 ) = ( 1.5 ) * ( 1 / 7 ) + ( 0.83 ) * ( 1 / 7 ) + ( 0.6 ) * ( 1 / 7 ) + ( 1.5 ) * ( 1 / 7 ) + ( 1 ) * ( 1 / 7 ) + ( 0.78 ) * ( 1 / 7 ) + ( 1.25 ) * ( 1 / 7 ) + ( 1.25 ) * ( 1 / 7 ) = 1.24$$

$$( fatigue degree of hips ) = ( ratio of P 2 to past average ) * ( weighting coefficient of P 2 ) + ( ratio of P 3 to past average ) * ( weighting coefficient of P 3 ) + ( ratio of P 4 to past average ) * ( weighting coefficient of P 4 ) + ( ratio of P 5 to past average ) * ( weighting coefficient of P 5 ) + ( ratio of P 6 to past average ) * ( weighting coefficient of P 6 ) + ( ratio of P 7 to past average ) * ( weighting coefficient of P 7 ) + ( ratio of P 9 to past average ) * ( weighting coefficient of P 9 ) + ( ratio of P 10 to past average ) * ( weighting coefficient of P 10 ) = ( 1.5 ) * ( 1 / 10 ) + ( 0.83 ) * ( 1 / 10 ) + ( 0.6 ) * ( 1 / 10 ) + ( 1.5 ) * ( 2 / 10 ) + ( 1 ) * ( 2 / 10 ) + ( 0.78 ) * ( 2 / 10 ) + ( 1.25 ) * ( 1 / 10 ) + ( 1.25 ) * ( 1 / 10 ) = 1.20$$

$$( fatigue degree of legs ) = ( ratio of P 7 to past average ) * ( weighting coefficient of P 7 ) = ( 0.78 ) * ( 1 / 1 ) = 0.78$$

As a result of the calculation, the fatigue degree is high as a whole except for the legs, as compared with ordinary days. Particularly, the fatigue degree of the non-dominant arm is high. This is because the user cleaned a high place with his/her non-dominant arm.

The fatigue degree estimation unit 622 stores a fatigue degree estimation result for each part of the user individually in association with calculation period information and the value of each of the calculation parameters in the fatigue degree log information DB 626.

<Calculation of Fatigue Degree for Each Part>

FIG. 15E is a diagram illustrating an example of a table 150E, in which a degree of concurrency of household chores, and an increment value are mapped to each other. The degree of concurrency indicates the number of household chores to be handled concurrently. As illustrated in FIG. 15E, when the user does two household chores concurrently, the fatigue degree of a mental part is 1.5 times of the fatigue degree when the user does one household chore. In the log information illustrated in FIG. 13, a fatigue degree of a mental part is calculated as follows.

$$( fatigue degree of mental part ) = ( ratio of P 1 to past average ) * ( weighting coefficient of P 1 ) * ( increment for concurrency degree ) + ( ratio of P 8 to past average ) * ( weighting coefficient of P 8 ) * ( increment for cocurrency degree ) = ( 2 ) * ( 3 / 4 ) * ( 1.5 ) + ( 1.1 ) * ( 1 / 4 ) * ( 1.5 ) = 2.67$$

As a result of the calculation, the fatigue degree of a mental part is high. This is because the user has done multiple household chores concurrently.

<Output of Fatigue Degree>

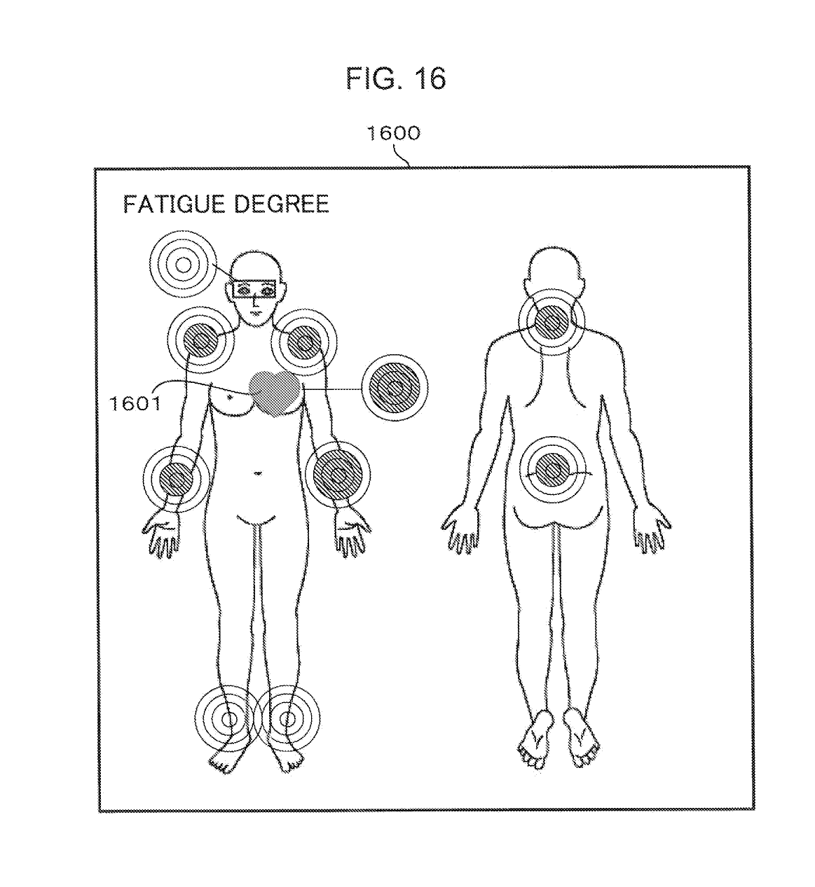

FIG. 16 is a diagram illustrating an example of a notification image 1600 indicating a fatigue degree estimation result by the fatigue degree estimation unit 622, which is notified to at least one of the user and a person related to the user. In the example of FIG. 16, a fatigue degree of each part is indicated by the size of a circle shown by a hatched portion. Further, the circle corresponding to a heart mark 1601 indicates a fatigue degree of a mental part. As the fatigue degree increases, the radius of a circle shown by a hatched portion stepwise increases.

When the notification image 1600 is generated, the fatigue degree estimation unit 622 may transmit the notification image 1600 to the terminal 603 of at least one of the user and a person related to the user, and may notify the estimation result.

The example of FIG. 16 illustrates a fatigue degree by the size of a circle shown by a hatched portion. This is merely an example. A fatigue degree may be expressed by changing the color of a circle or changing the density of a circle in accordance with the fatigue degree.

As described above, in the fatigue degree estimating system 600 according to the first embodiment, it is possible to estimate a fatigue degree of the user in doing the household chores for each part of the user, considering not only a direct operation of the user with respect to the device 601, but also movement of the user before and after the direct operation. This makes it possible to provide the user with detailed fatigue information. Further, it is also possible to estimate a fatigue degree of a mental part of the user. This is advantageous in providing the user with a fatigue degree physically and mentally.

The first embodiment has been described as above. Utilizing, in addition to log information, information such as personal information, position information, utterance information, facial expression information, and schedule information of a household worker, and collecting a continuous state of a fatigue degree is advantageous in estimating a fatigue degree of the household worker with high precision.

### Second Embodiment

The fatigue degree estimating system according to the second embodiment has a feature such that a message of soothing, appreciation, or praise is notified to the user in doing household chores.

<System Configuration of Second Embodiment>

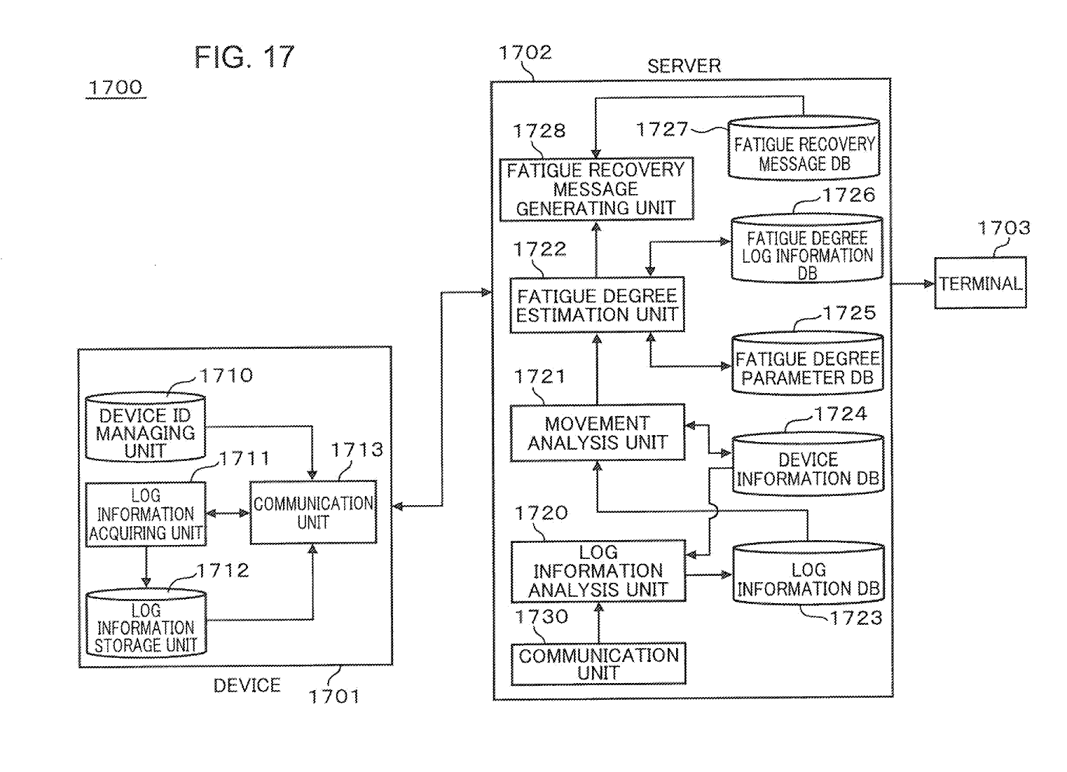

FIG. 17 is a block diagram illustrating an example of a configuration of a fatigue degree estimating system 1700 according to the second embodiment of the invention.

As illustrated in FIG. 17, the fatigue degree estimating system 1700 is provided with a device 1701, a server 1702, and a terminal 1703.

The device 1701 and the terminal 1703 have substantially the same configuration as the device 601 and the terminal 603 in the first embodiment. The server 1702 is provided with a fatigue recovery message generating unit 1728 and a fatigue recover message DB 1727 in addition to the element blocks in the server 602 in the first embodiment. The element blocks in the server 1702 other than the above are the same as the element blocks in the server 602 having the same reference numerals.

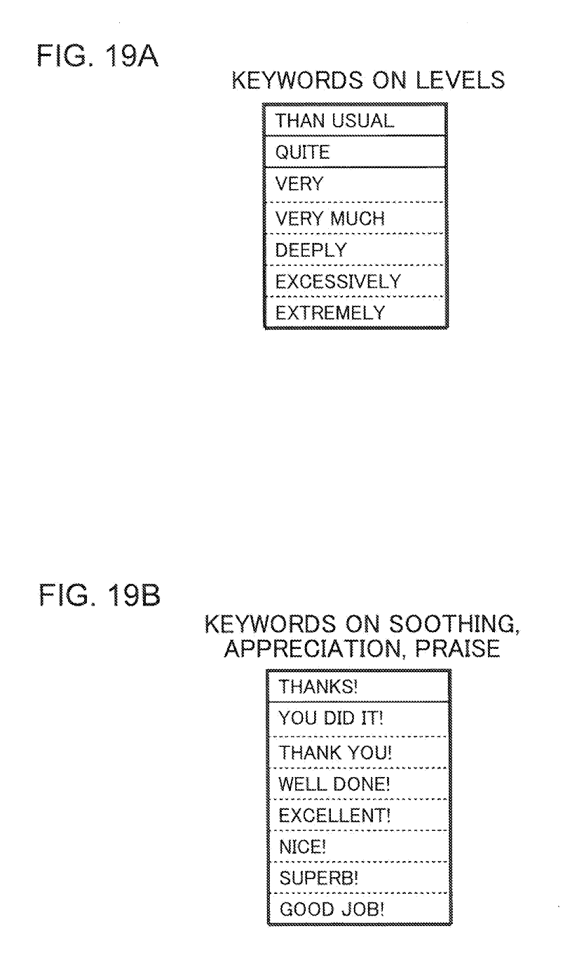

The fatigue recovery message generating unit 1728 is constituted of a software or a dedicated hardware circuit, for instance. The fatigue recovery message generating unit 1728 is configured to retrieve the fatigue recovery message DB 1727 from an estimation result by a fatigue degree estimation unit 1722, generate a message of soothing, appreciation, or praise relating to the fatigue of the user, and notify at least one of the user and a person related to the user of the message via a communication unit 1730. The fatigue recovery message DB 1727 is a DB configured to store messages of soothing, appreciation, or praise with respect to the fatigue of the user. Specifically, the fatigue recovery message DB 1727 stores keywords on levels as illustrated in FIG. 19A, and keywords on soothing, appreciation, and praise as illustrated in FIG. 19B.

<Processing of Generating Fatigue Recovery Message>

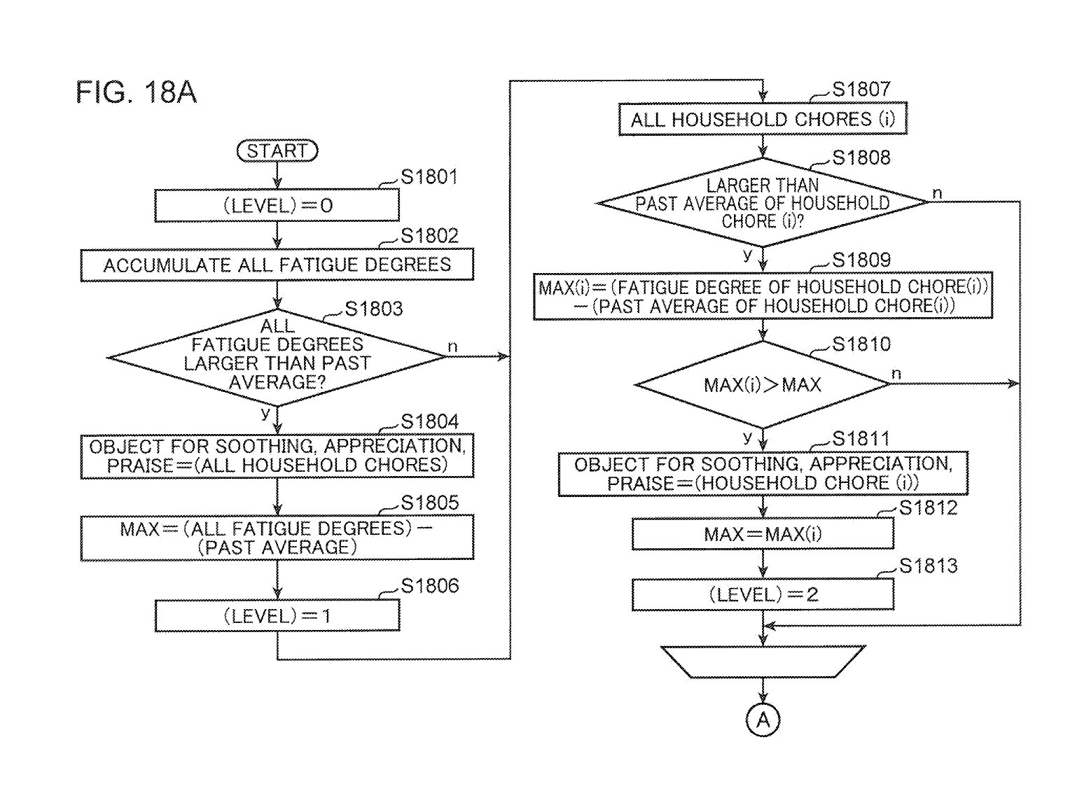

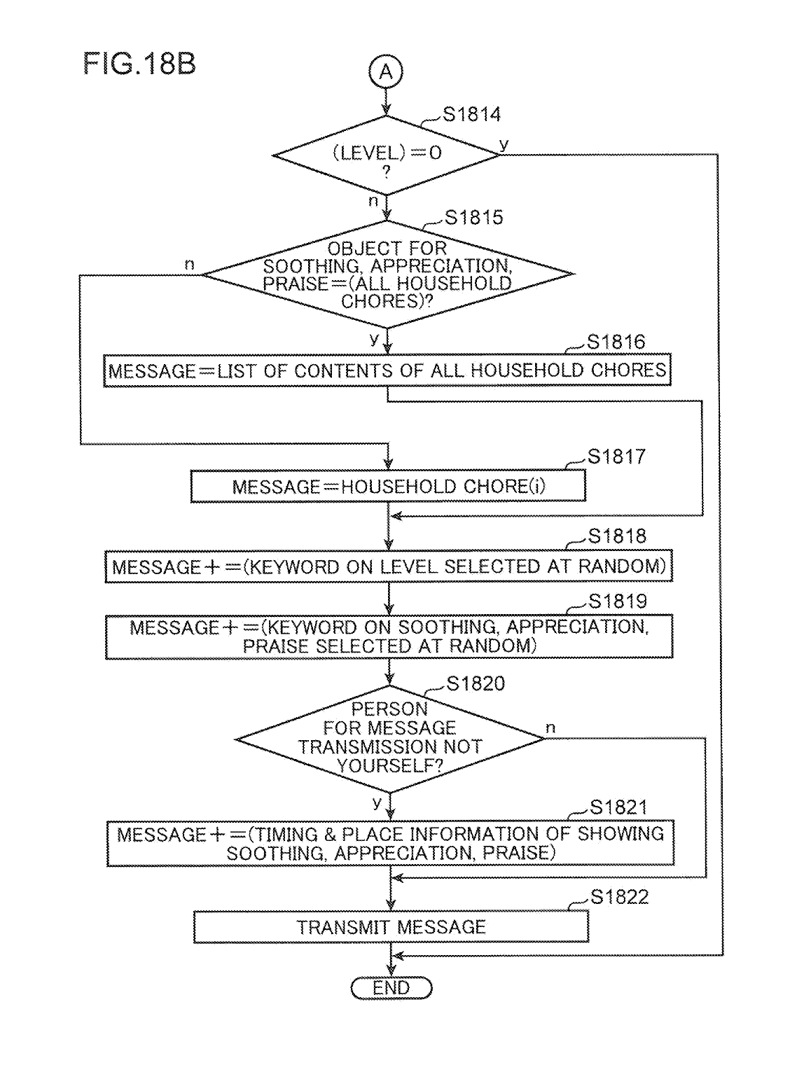

FIGS. 18A and 18B are a flowchart illustrating an example of processing to be performed by the fatigue recovery message generating unit 1728.

First of all, the level is initialized to zero (in Step S1801). Subsequently, a fatigue degree of each household chore is accumulated, and a total fatigue degree is calculated (in Step S1802). In this example, a fatigue degree of each household chore is defined by a sum of a fatigue degree of each part and a fatigue degree of a mental part, which is obtained from cleaner log information if the device is the cleaner, or is defined by a sum of a fatigue degree of each part and a fatigue degree of a mental part, which is obtained from washing machine log information if the device is the washing machine. Accordingly, assuming that the household chores are constituted of cleaning and washing, the sum of fatigue degrees concerning cleaning, and the sum of fatigue degrees concerning washing are calculated as a total fatigue degree.

Subsequently, it is determined whether the total fatigue degree on this day is larger than the past average (in Step S1803). When the total fatigue degree is larger than the past average (y in Step S1803), an object for which a message of soothing, appreciation, or praise is to be notified is set to all the household chores (in Step S1804). A value obtained by subtracting the past average from the total fatigue degree is set to MAX (in Step S1805), and the level is set to 1 (in Step S1806).

In Step S1803, when the total fatigue degree is not larger than the past average in Step S1803 (n in Step S1803), or after the processing of Step S1806 is ended, the processing of Step S1808 to Step S1813 is repeated for all the household chores (i) (in Step S1807).

The processing of Step S1807 to Step S1813 is described. First of all, it is determined whether the fatigue degree of the household chore (i) is larger than the past average of the household chore (i) (in Step S1808). When the fatigue degree of the household chore (i) is larger than the past average (y in Step S1808), a value obtained by subtracting the past average of the fatigue degree of the household chore (i) from the fatigue degree of the household chore (i) is set to MAX(i) (in Step S1809).

Subsequently, when MAX(i) is larger than MAX (y in Step S1808), an object for which a message of soothing, appreciation, or praise is to be notified is set to the household chore (i) (in Step S1811). Subsequently, MAX(i) is set in MAX (in Step S1812), and the level is set to 2 (in Step S1813).

On the other hand, in Step S1808, when the fatigue degree of the household chore (i) is not larger than the past average of the household chore (i) (n in Step S1808), or in Step S1810, when Max(i) is not larger than MAX (n in Step S1810), the processing returns to Step S1808, and the same processing is repeated for the next household chore (i+1).

When the processing of Step S1807 is ended for all the household chores (i), it is determined whether the level is zero (in Step S1814). In Step S1814, when the level is not zero (n in Step S1814), it is determined whether an object for which a message of soothing, appreciation, or praise is to be notified is all the household chores (in Step S1815). In this case, when the level is 1, an object for which the message is to be notified is determined to be all the household chores (y in Step S1815), and a text such that the contents of all the household chores are expressed in the form of a list is set as a message (in Step S1816).

In Step S1815, an object for which a message of soothing, appreciation, or praise is to be notified is not all the household chores in Step S1815 (n in Step S1815), the text on the household chore (i) is set as a message (in Step S1817). In this example, when the level is 2, the determination result is negative in Step S1815. In this example, the text on the household chore (i) set to MAX in Step S1812 is set as a message. For instance, assuming that the household chores are washing and cleaning, and the household chore (i) of washing is set to MAX in Step S1812, the text on washing is set as a message.

In Step S1818, a keyword is extracted at random from the keywords on levels described in FIG. 19A, and the extracted keyword is added to the message set in Step S1816 or in Step S1817 (in Step S1818). FIG. 19A is a diagram illustrating an example of the keywords on levels. FIG. 19A describes the keywords on levels such as “THAN USUAL”, “QUITE”, and “VERY”.

In Step S1819, a keyword is extracted at random from the keywords of soothing, appreciation, praise described in FIG. 19B, and the extracted keyword is added to the message (in Step S1819). FIG. 19B is a diagram illustrating an example of a keyword table on soothing, appreciation, or praise. FIG. 19B describes the keywords on soothing, appreciation, or praise such as “THANKS”, “YOU DID IT”, and “THANK YOU”.

After Step S1819 is ended, it is determined whether a person for message transmission is the user himself or herself (in Step S1820). When a person for message transmission is not the user himself or herself (y in Step S1820), a timing of uttering a message of soothing, appreciation, or praise, and place information of uttering a message are added to the message. Subsequently, the set message is transmitted to the terminal 1703 of a person related to the user himself or herself (a household worker) (in Step S1822).

In this example, assuming that the household worker is the wife, and the related person is the husband, a timing of uttering a message indicates a timing at which the husband actually utters a message of soothing, appreciation, or praise to the wife. Specifically, a time when the husband comes back home from work, or a time when supper is over is set as the timing of uttering a message. This timing is extracted at random from candidates of a predetermined timing, for instance.

Further, the place information of uttering a message indicates a place where the husband is supposed to actually utter a message of soothing, appreciation, or praise to the wife. Specifically, examples of the place information are places at home such as a corridor or an entrance hall. The place information is also extracted at random from candidates of predetermined place information.

In Step S1820, when the person for message transmission is the user himself or herself (n in Step S1820), Step S1821 is skipped, and the message is transmitted to the user himself or herself.

In Step S1814, when the level is zero (y in Step S1824), the processing is ended without transmitting a message. A message indicating no message may be transmitted to the person for message transmission.

<Result Notification to User>

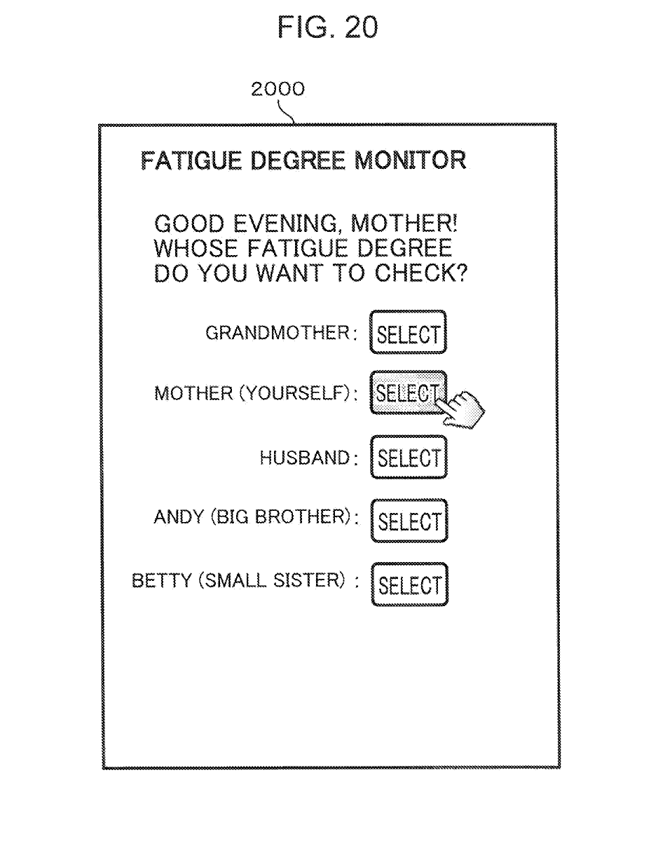

FIG. 20 is a diagram illustrating an example of a fatigue degree check image 2000 in the fatigue degree estimating system according to the second embodiment. FIG. 20 illustrates the check image 2000 when the user is mother. In this case, the check image 2000 displays “GOOD EVENING, MOTHER!OF WHOM DO YOU WANT TO CHECK FATIGUE DEGREE?”Further, the check image 2000 displays the family members of mother as a list. Selecting one of the members allows for mother to check the fatigue degree of the selected family member.

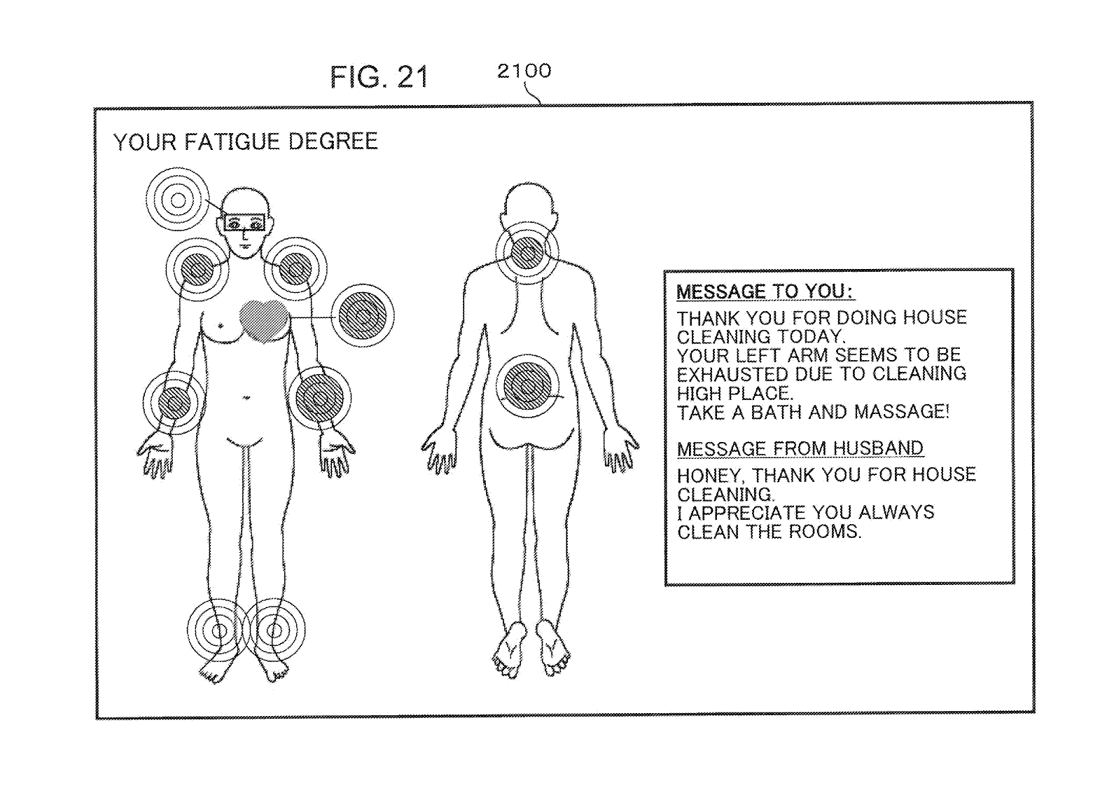

When mother as the user selects her own column in the check image 2000, a notification image 2100 indicating a fatigue degree estimation result of mother illustrated in FIG. 21 is displayed on the terminal 1703 of mother. This allows for mother to check the fatigue degree of herself by the household chores.

FIG. 21 is a diagram illustrating an example of the notification image 2100 indicating the fatigue degree of the household worker. In FIG. 21, on the left side of the notification image 2100, there are displayed a fatigue degree of each part and a fatigue degree of a mental part of the household worker. The fatigue degree display manner is substantially the same as illustrated in FIG. 16 except for the point that the fatigue degree of the back side of the person is also displayed in addition to the fatigue degree of the front side of the person. Further, on the right side of the notification image 2100, there is displayed a message of soothing, appreciation, or praise. The message is the message set in Step S1819 in FIG. 18B, or a message including some modification to the message set in Step S1819. In this example, as “MESSAGE FOR YOU”, for instance, there is displayed a message “THANK YOU FOR DOING HOUSE CLEANING TODAY. YOUR LEFT ARM SEEMS TO BE EXHAUSTED DUE TO CLEANING HIGH PLACE. TAKE A BATH AND MASSAGE!”.

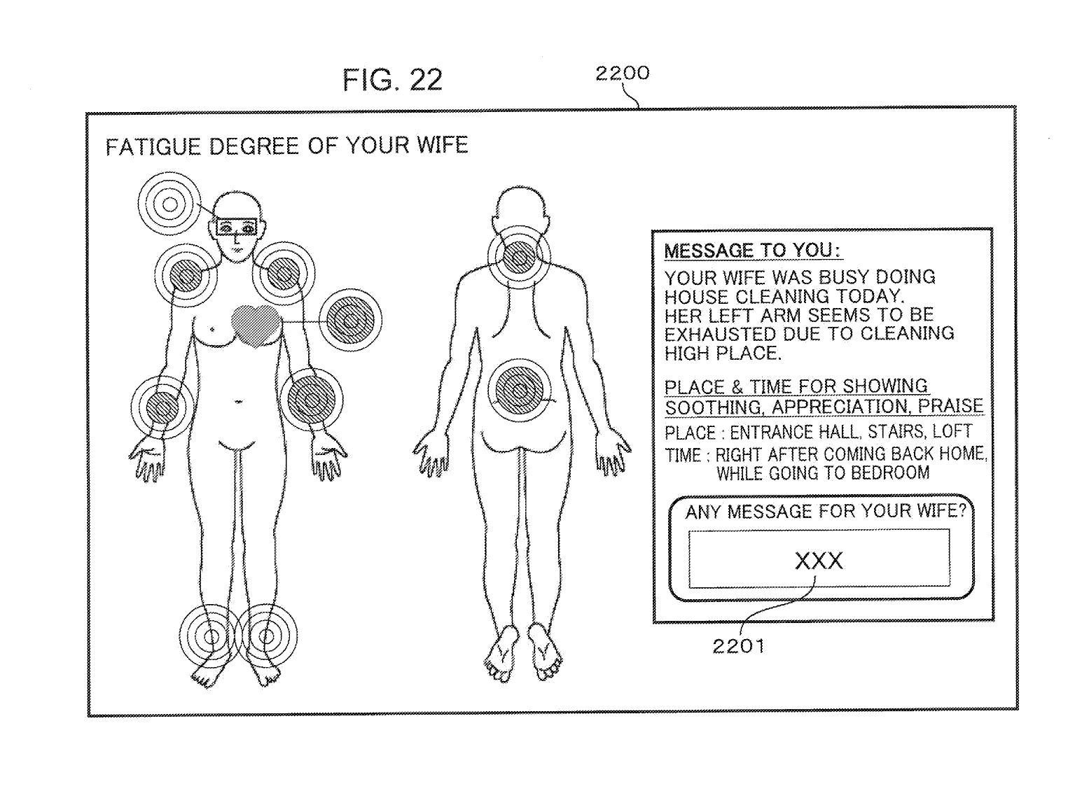

Further, “MESSAGE FROM HUSBAND” is displayed below “MESSAGE FOR YOU”, and a message of soothing, appreciation, or praise is displayed from the related person to the household worker. The message from the related person is the message that has been input when the husband, who is the related person, checked a notification image 2200 (see FIG. 22) of mother. This makes it possible to increase the effect of soothing, appreciation, or praise to the household worker in doing the household chores. In particular, an effect on mental fatigue is expected.

FIG. 22 is a diagram illustrating an example of the fatigue degree notification image 2200 when the related person checks the fatigue degree of the household worker. The notification image 2200 is displayed on the display of the terminal 1703 of the husband, who is the related person.

In FIG. 22, on the left side of the notification image 2200, there are displayed a fatigue degree of each part and a fatigue degree of a mental part of mother (wife), who is the household worker. The fatigue degree display manner is the same as in FIG. 21. Further, on the right side of the notification image 2200, there are displayed a message of soothing, appreciation, or praise which the husband as the related person is supposed to utter to the wife as the household worker, and the place and the time the message is supposed to be uttered. The place and the time are set in Step S1821 in FIG. 18B.

In the notification image 2200, a message input column 2201, in which a message is notified from the related person to the household worker, is formed at a position below the message display column. The related person operates the terminal 1703 of his or her own, and inputs a message of soothing, appreciation, or praise to the household worker in the message input column 2201. The input message is displayed in the message display column of the notification image 2100 of the household worker illustrated in FIG. 21. This allows for the related person to promptly utter a message of soothing, appreciation, or praise to the household worker.

As described above, in the fatigue degree estimating system according to the second embodiment, a message of soothing, appreciation, or praise for doing the household chores is notified to the household worker. This makes it possible to recover the fatigue of the household worker from a mental approach.

### Third Embodiment

The fatigue degree estimating system according to the third embodiment has a feature such that a fatigue recovery tip using a fatigue recovery device is proposed to the user.

<System Configuration of Third Embodiment>

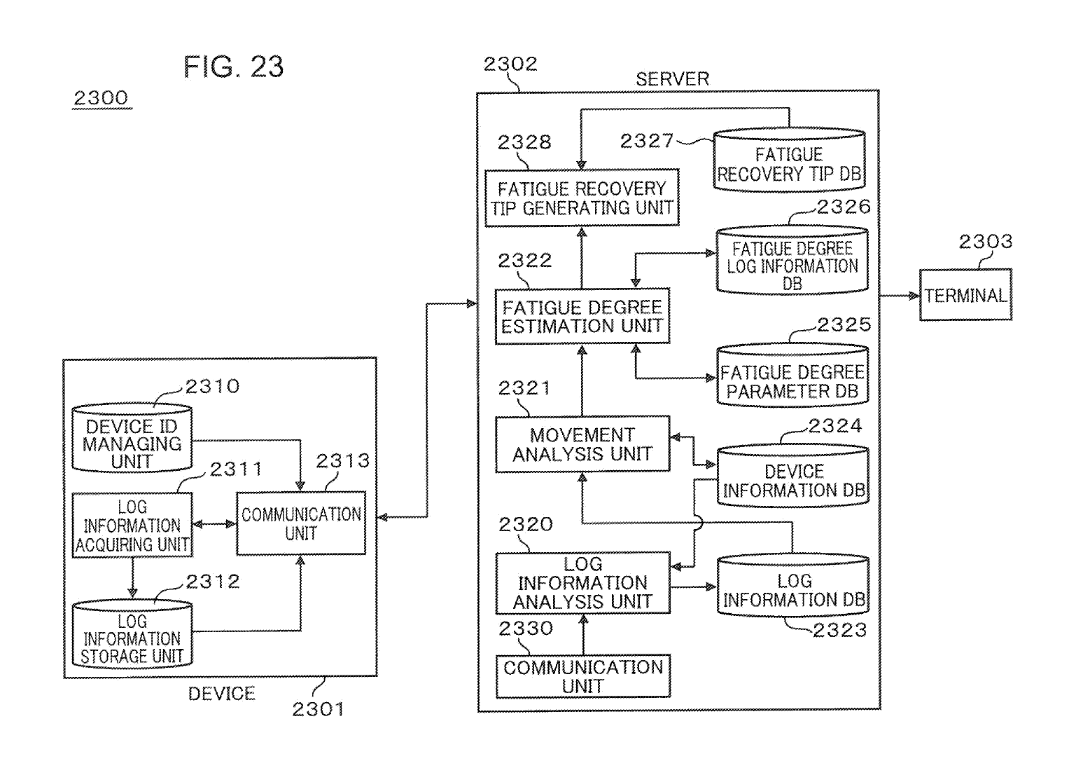

FIG. 23 is a block diagram illustrating an example of a configuration of a fatigue degree estimating system 2300 according to the third embodiment of the invention.

As illustrated in FIG. 23, the fatigue degree estimating system 2300 is provided with a device 2301, a server 2302, and a terminal 2303.

The device 2301 and the terminal 2303 have substantially the same configuration as the device 601 and the terminal 603 in the first embodiment. The server 2302 is provided with a fatigue recovery tip generating unit 2328 and a fatigue recover tip DB 2327 in addition to the element blocks in the server 602 in the first embodiment. The element blocks in the server 2302 other than the above are the same as the element blocks in the server 602 having the same reference numerals.

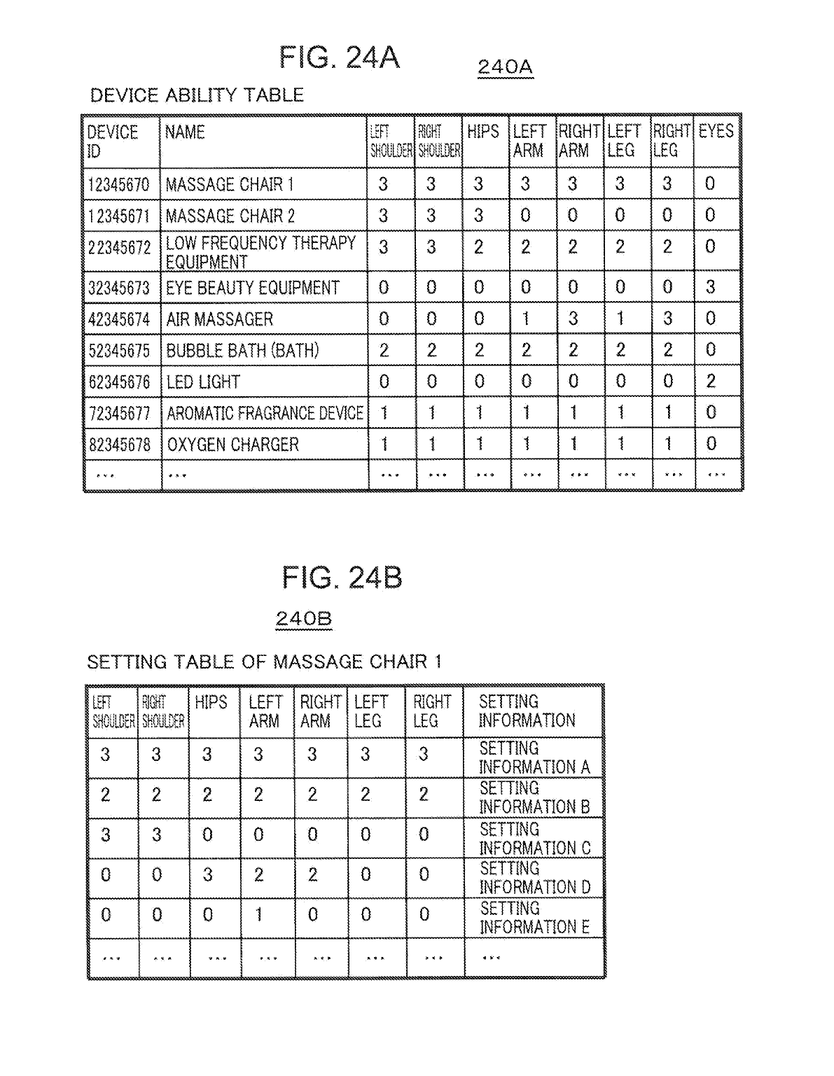

The fatigue recovery tip DB 2327 is a DB configured to store the fatigue recovery ability of each of the fatigue recovery devices with respect to fatigue of the user, and setting information of each of the fatigue recovery devices. Specifically, the fatigue recovery tip DB 2327 is configured to store a device ability table 240A illustrated in FIG. 24A and a setting table 240B illustrated in FIG. 24B. The device ability table 240A stores device ability information which defines the fatigue recovery ability of each part with respect to each of the fatigue recovery devices owned by the user.

In the device ability table 240A, the device ID is a unique identifier given to a corresponding fatigue recovery device, and the name is the name of the corresponding fatigue recovery device. The device ability table 240A stores the fatigue recovery ability of each of the fatigue recovery devices for each part. The value of fatigue recovery ability indicates that fatigue expressed by a fatigue degree of not larger than the value can be recovered. For instance, when the fatigue recovery ability is “3”, it is possible to recover from fatigue up to the fatigue degree “3”.

In the setting table 240B, there is registered setting information of a fatigue recovery device, in which setting contents suitable for the fatigue degree of each body part is defined. In FIG. 24B, there is illustrated an example of a massage chair setting table 240B. Actually, in the fatigue recovery tip DB 2327, there is registered a setting table 240B of each of the fatigue recovery devices registered in the device ability table 240A.

In the setting table 240B, there is defined a fatigue recovery ability of each body part by using a massage chair in each of the setting information A to E. For instance, when the setting information A is set, the massage chair is capable of recovering from fatigue of each body part whose fatigue degree is not larger than 3. When the setting information B is set, the massage chair is capable of recovering from fatigue of each body part whose fatigue degree is not larger than 2.

<Processing of Generating Fatigue Recovery Tip>

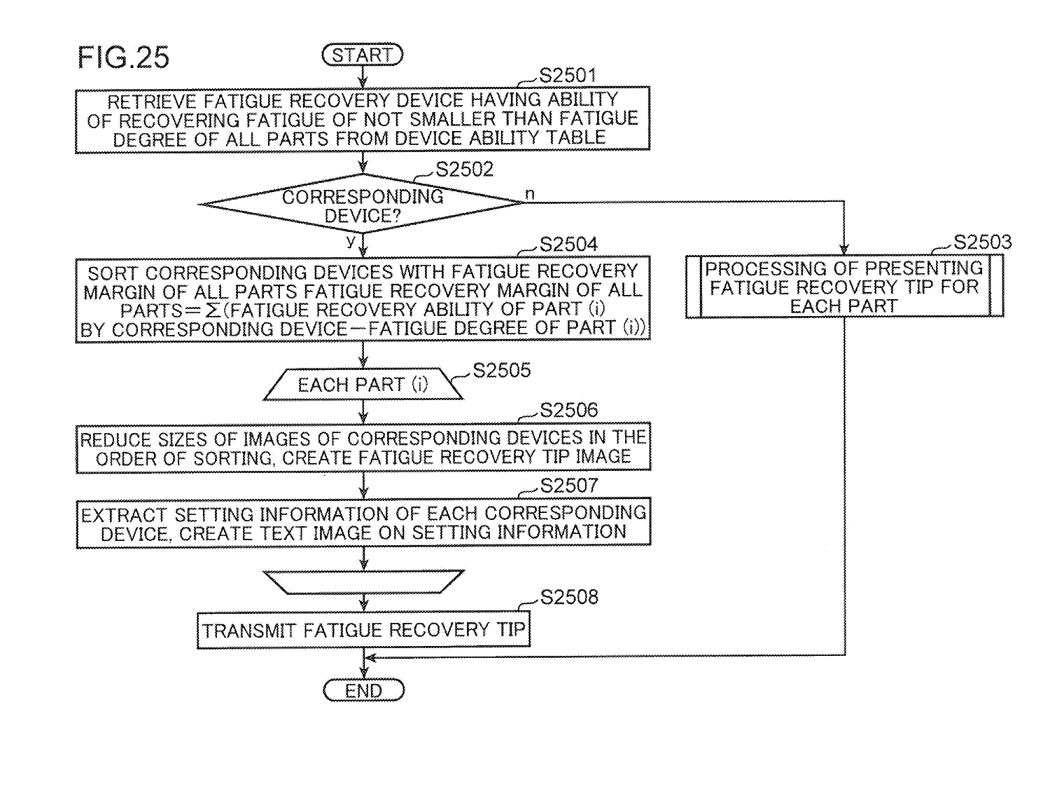

FIG. 25 is a flowchart illustrating an example of processing to be performed by the fatigue recovery tip generating unit 2328. First of all, the device ability table 240A is referred to, and one or more fatigue recovery devices having a fatigue recovery ability capable of recovering from fatigue of not smaller than the fatigue degree of the user is retrieved for each of all the parts (in Step S2501).

As a result of Step S2501, when a corresponding fatigue recovery device (corresponding device) is not detected (n in Step S2502), the processing is proceeded to processing of presenting a fatigue recovery tip for each part (in Step S2503). On the other hand, when a corresponding device is detected (y in Step S2502), a fatigue recovery margin of all the parts is calculated as expressed by the following formula (4) with respect to each of the detected corresponding devices, and the corresponding devices are sorted in the order of decreasing the fatigue recovery margin (in Step S2504). The processing of presenting a fatigue recovery tip for each part in Step S2503 is described later.

$$\begin{matrix} [ Formula 4 ] & \\ ( fatigue recovery margin of all parts ) = {\sum }_{i} ( fatigue recovery ability of part ( i ) with respect to corresponding device ) - ( fatigue degree of part ( i ) ) & ( 4 )\end{matrix}$$

In this example, the fatigue recovery margin of part (i) indicates a margin, of the fatigue ability recovery of the part (i) with respect to the corresponding device, with respect to the fatigue degree of the part (i) of the user. Further, the fatigue recovery margin of all parts is a sum of fatigue recovery margins of the parts. Accordingly, as the corresponding device has a larger value of the fatigue recovery margin of all parts, the fatigue recovery effect to the user is high.

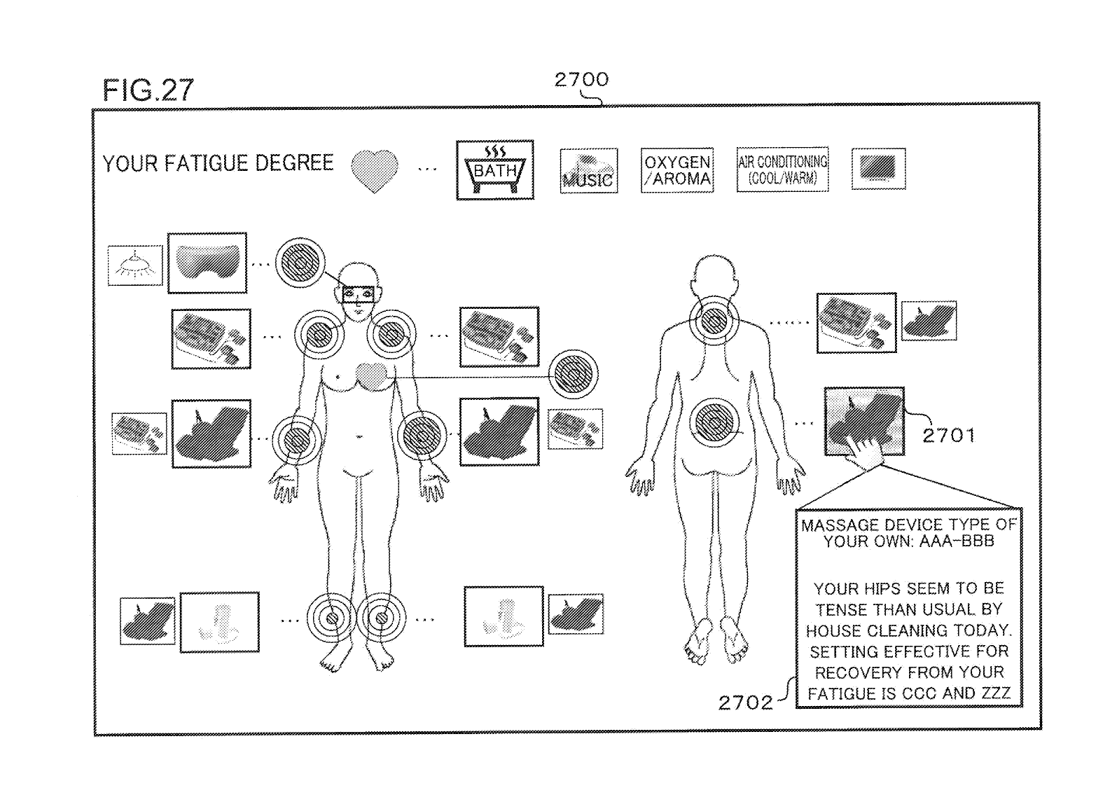

After Step S2504 is ended, the processing of Steps S2506 and S2507 is repeated for each part (i) (in Step S2505). First of all, the images indicating corresponding devices are reduced in size in the order of sorting in Step S2504, a designated number of (e.g. two) corresponding devices are arranged in association with the part (i), and a fatigue recovery tip image 2700 (see FIG. 27) is created (in Step S2506).

In the example of FIG. 27, the reason why the fatigue recovery effect of the right arm of the user is high is that the fatigue recovery devices are used in the order of a massage chair, and a low-frequency therapy equipment. The image of the low-frequency therapy equipment is displayed with a smaller size than the size of the image of the massage chair, and the images of the massage chair and of the low-frequency therapy equipment are displayed side by side in association with an image of the right arm.

Subsequently, setting information suitable for the fatigue degree of the user is extracted from the setting table 240B with respect to each of the corresponding devices arranged in the fatigue recovery tip image 2700, and a text image on the extracted setting information is created (in Step S2507).

In the example of FIG. 24B, setting information extracting processing is described, assuming that the parts are the left shoulder, the right shoulder, and the hips. Assuming that the fatigue degree of the left shoulder of the user is “2”, the setting information in which the fatigue recovery ability of the left shoulder is not smaller than “2” is the setting information A, B, and C. Accordingly, these three setting information is extracted. Next, assuming that the fatigue degree of the right shoulder of the user is “3”, the setting information B is removed, because the fatigue recovery ability of the right shoulder is “2” in the setting information B. Next, assuming that the fatigue degree of the hips of the user is “2”, the setting information C is removed because the fatigue recovery ability of the hips is “0” in the setting information C. In this way, finally, the setting information A is extracted.

As described above, setting information is extracted in the order of decreasing the number of parts of which the fatigue degree of the user can be recovered. In the case where there is two or more setting information indicating that it is possible to recover the fatigue degree of all the parts of the user, the two or more setting information may be extracted.

When the processing of Step S2505 is ended for all the parts (i), the fatigue recovery tip image 2700 is transmitted to the terminal 2303 of the user as a fatigue recovery tip (in Step S2508), and the processing is ended. As will be described later, the text image on setting information created in Step S2507 is transmitted in response to interaction from the user in the fatigue recovery tip image 2700. This is merely an example. The text image on setting information created in Step S2508 may be transmitted to the user along with the fatigue recovery tip image 2700.

<Processing of Presenting Fatigue Recovery Tip for Each Part>

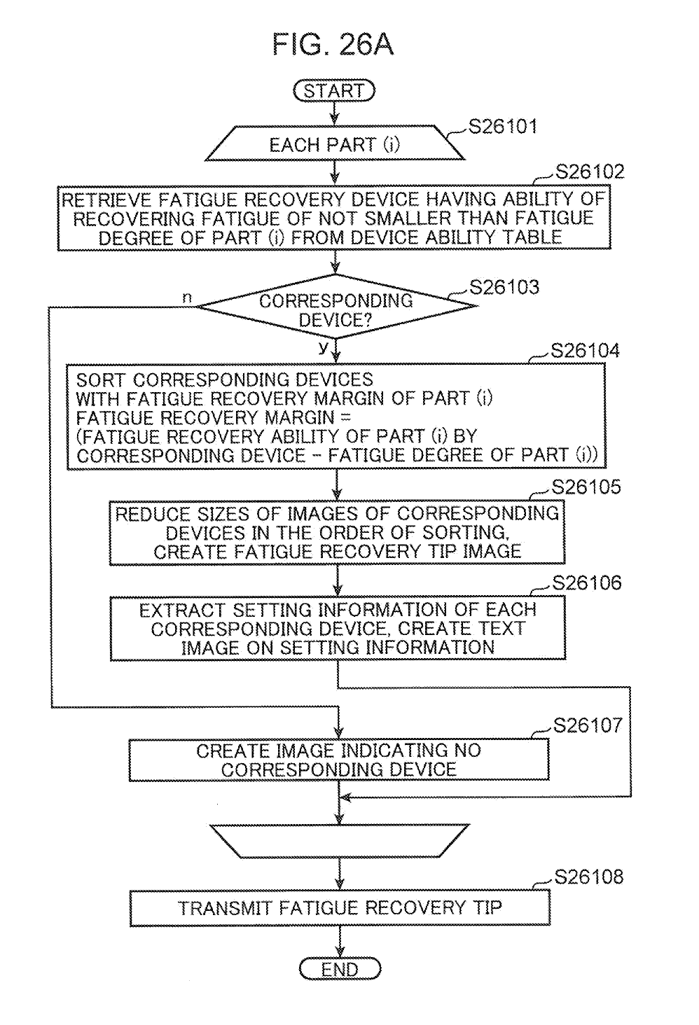

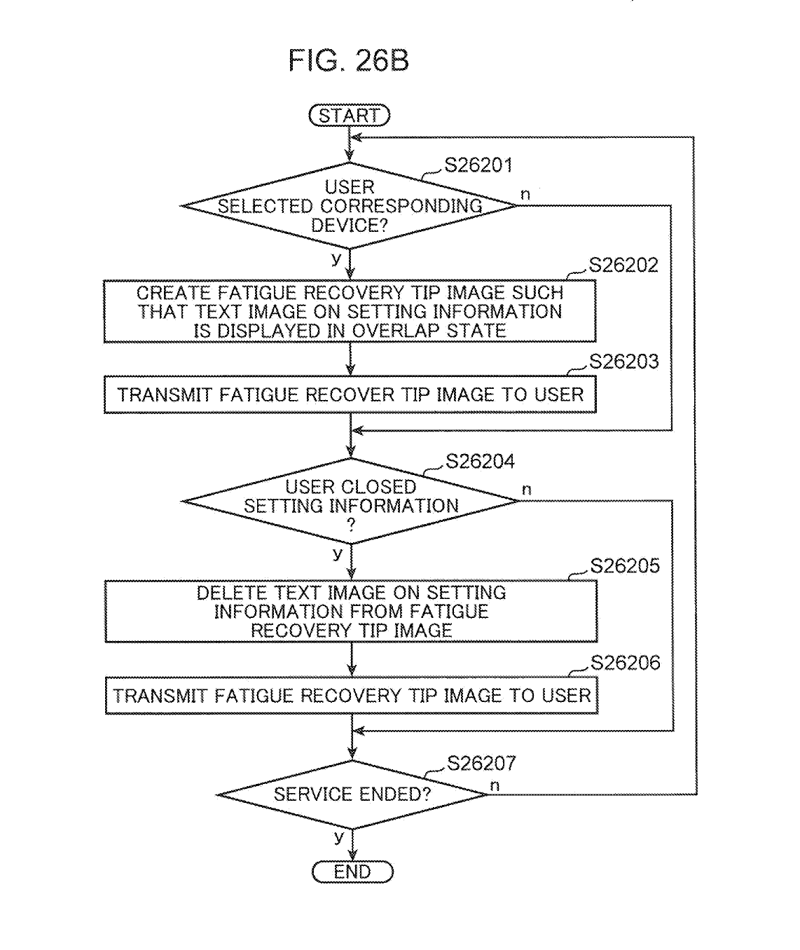

FIG. 26A is a flowchart illustrating an example of processing of presenting a fatigue recovery tip for each part (Step S2503 in FIG. 25).

First of all, processing of Steps S26102 to S26107 is repeated for each part (i) (in Step S26101).

The device ability table 240A illustrated in FIG. 24A is referred to, and one or more fatigue recovery devices having a fatigue recovery ability capable of recovering from fatigue of not smaller than the fatigue degree are retrieved (in Step S26102). As a result of Step S26102, when corresponding fatigue recovery devices (corresponding devices) are detected (y in Step S26103), as expressed by the following formula (5), a fatigue recovery margin is calculated for each part (i), and the corresponding devices are sorted in the order of decreasing the fatigue recovery margin (in Step S26104). Subsequently, the images of the corresponding devices are reduced in size in the order of sorting, a designated number of corresponding devices are arranged in association with the part (i), and the fatigue recovery tip image 2700 is created (in Step S26105). Subsequently, setting information suitable for the fatigue degree of the user is extracted from the setting table with respect to each of the corresponding devices arranged in the fatigue recovery tip image 2700, and a text image on the extracted setting information is created (in Step S26106).

[Formula 5]

(fatigue recovery margin)={(fatigue recovery ability of part (i) with respect to corresponding device)−(fatigue degree of part (i))} (5)

In this example, there is extracted setting information on the fatigue recovery ability capable of recovering from fatigue of not smaller than the fatigue degree of the user for the corresponding part (i). For instance, in the example of FIG. 24B, assuming that the fatigue degree of the left shoulder of the user is “2”, the setting information A, B, and C whose fatigue recovery ability is not smaller than “2” is extracted.

In Step S26103, when a corresponding device is not detected (n in Step S26103), there is created an image or a text image indicating that there is no fatigue recovery device capable of recovering from fatigue (in Step S26107).

When the processing of Step S26101 is ended for all the parts (i), the fatigue recovery tip image 2700 or an image indicating no fatigue recovery device is transmitted to the terminal 2303 of the user, as a fatigue recovery tip (in Step S26108), and the processing is ended. As will be described later, the text image on setting information created in Step S26106 is transmitted in response to interaction from the user in the fatigue recovery tip image 2700. This is merely an example. The text image on setting information created in Step S26108 may be transmitted to the user along with the fatigue recovery tip image 2700.

<UI Processing Flow>

FIG. 26B is a flowchart illustrating an example of user interface (UI) processing in a fatigue recovery system 2800 according to the third embodiment. Further, FIG. 27 is a diagram illustrating an example of the fatigue recovery tip image 2700 in the third embodiment. In FIG. 27, a massage chair 2701 is presented as a corresponding device to be used in a fatigue recovery tip for the hips, and the massage chair 2701 is selected by the user. A text image on massage chair setting information suitable for recovery from fatigue of the user's hips is displayed.

In the following, the flowchart of FIG. 26B is described referring to FIG. 27. First of all, when a fatigue recovery device is selected by the user from among the fatigue recovery devices displayed in a row in association with each part in FIG. 27 (y in Step S26201), the fatigue recovery tip image 2700 such that a text image on setting information of the selected fatigue recovery device (corresponding device) is displayed in an overlap state is created (in Step S26202), and is transmitted to the user (in Step S26203).

In Step S26201, when a corresponding device is not selected by the user (n in Step S26201), the processing is proceeded to Step S26204. When an operation of closing the setting information is input by the user (y in Step S26204), the text image on setting information of the corresponding device is deleted from the fatigue recovery tip image 2700 (in Step S26205), and the fatigue recovery tip image is transmitted to the terminal 2303 of the user (in Step S26206).

In Step S26204, when an operation of closing the setting information is not input by the user (n in Step S26204), the processing is proceeded to Step S26207.

Subsequently, when an operation of ending the service by the fatigue degree recovering system is input by the user (y in Step S26207), the processing is ended. When an operation of ending the service is not input (n in Step S26207), the processing returns to Step S26201, and the UI processing is continued.

As described above, in the fatigue degree recovering system according to the third embodiment, a fatigue recovery device suitable for recovery from the fatigue of the user is notified to the user from among the fatigue recovery devices owned by the user. This allows for the user to effectively utilize the fatigue recovery devices owned by the user. This is advantageous in guiding the user for recovery from fatigue in a more rational way. Further, the images of the fatigue recovery devices suitable for recovery from fatigue are displayed with different sizes in the order of decreasing the fatigue recovery effect in the fatigue recovery tip image 2700. This allows for the user to recognize a fatigue recovery device having a high fatigue recovery effect at a glance.

Further, in the fatigue recovery tip image 2700, there are displayed the images of fatigue recovery devices suitable for recovery from fatigue individually for each part. This allows for the user to select a fatigue recovery device suitable for each part. This is advantageous in avoiding a likelihood that the user may use a fatigue recovery device inappropriate for recovery from fatigue, and may damage the body of the user.

Further, when an image of a fatigue recovery device is selected in the fatigue recovery tip image 2700, there is displayed a text image on setting information of the selected fatigue recovery device, which is suitable for recovery from fatigue of the user. This allows for the user to properly perform setting on the selected fatigue recovery device. As a result, the user is smoothly guided to recover from fatigue. Further, the text image on setting information is displayed in response to interaction from the user. Accordingly, as compared with a configuration, in which a text image on setting information of all the fatigue recovery devices is displayed in the fatigue recovery tip image 2700 from the beginning, this is advantageous in enhancing the visual recognition of the fatigue recovery tip image 2700.

In the fatigue recovery system according to the third embodiment, the fatigue recovery tip image 2700 includes setting information on a fatigue recovery device. Alternatively, for instance, the fatigue recovery tip image 2700 may also include information on a use time of a fatigue recovery device, or side effects resulting from use of the fatigue recovery device. Further, when the user does not own a fatigue recovery device suitable for recovery from fatigue, the fatigue recovery tip image 2700 may include information introducing a fatigue recovery device capable of recovering from fatigue.

### Fourth Embodiment

A fatigue degree estimating system according to the fourth embodiment has a feature such that a fatigue recovery device is remote controlled with use of setting information on the fatigue recovery device suitable for recovery from fatigue of the user.

<System Configuration of Fourth Embodiment>

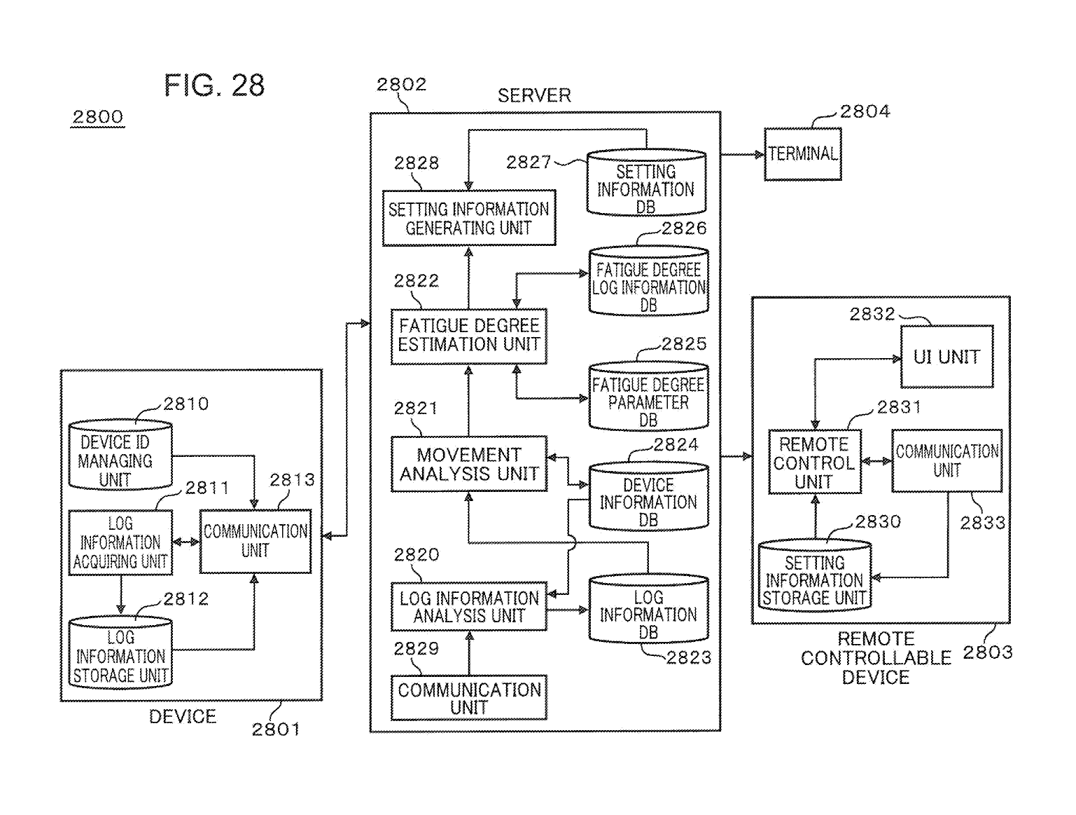

FIG. 28 is a block diagram illustrating an example of a configuration of a fatigue degree estimating system 2800 according to the fourth embodiment of the invention

As illustrated in FIG. 28, the fatigue degree estimating system 2800 is provided with a device 2801, a server 2802, a remote controllable device 2803, and a terminal 2804.

The device 2801 and the terminal 2804 have substantially the same configuration as the device 601 and the terminal 603 in the first embodiment.

The remote controllable device 2803 is a remote controllable fatigue recovery device owned by the user. The remote controllable device 2803 is provided with a setting information storage unit 2830, a remote control unit 2831, a UI unit 2832, and a communication unit 2833.

The communication unit 2833 is constituted of a communication circuit for connecting the remote controllable device 2803 to a public communication network such as the Internet, and is configured to receive setting information to be transmitted from the server 2802 and store the received setting information in the setting information storage unit 2830.

The setting information storage unit 2830 is constituted of e.g. a non-volatile memory, and is configured to store setting information to be transmitted from the server 2802 in accordance with the fatigue degree of the user individually for each user.

The remote control unit 2831 is constituted of a software or a dedicated hardware circuit, for instance, and is configured to read setting information of the user from the setting information storage unit 2830, and control the remote controllable device 2803 in accordance with the read setting information.

The UI unit 2832 is constituted of a software or a dedicated hardware circuit, for instance, and is configured to display an image (UI image) serving as a user interface on an unillustrated display, on the display of the remote controllable device 2803. Further, the UI unit 2832 is configured to receive an operation from the user, and notify the received operation to the remote control unit 2831.

The server 2802 is further provided with a setting information generating unit 2828 and a setting information DB 2827 in addition to the element blocks in the server 602 in the first embodiment. The element blocks in the server 2802 other than the above are the same as the element blocks in the server 602 having the same reference numerals.

The setting information generating unit 2828 is constituted of a software or a dedicated hardware circuit. The setting information generating unit 2828 is configured to select a remote controllable device 2803 suitable for recovery from fatigue of the user, with use of a fatigue degree estimation result by a fatigue degree estimation unit 2822 and information stored in the setting information DB 2827, and generate setting information suitable for recovery from fatigue of the user in the selected remote controllable device 2803. The setting information generating unit 2828 is configured to transmit the generated setting information to the remote controllable device 2803 via a communication unit 2829.

The setting information DB 2827 is a DB configured to store the fatigue recovery ability of each of the remote controllable devices 2803 with respect to fatigue of the user, and setting information of each of the remote controllable devices 2803. Specifically, the setting information DB 2827 is configured to store a device ability table 240A as illustrated in FIG. 24A and a setting table 240B as illustrated in FIG. 24B. In the embodiment, the device ability table 240A has substantially the same data construction as the device ability table 240A in the third embodiment, but is different from the device ability table 240A in the third embodiment in a point that the registered fatigue recovery devices are the remote controllable devices 2803 owned by the user.

Further, in the embodiment, the setting table 240B has substantially the same data construction as the setting table 240B in the third embodiment.

<Setting Information Generation>

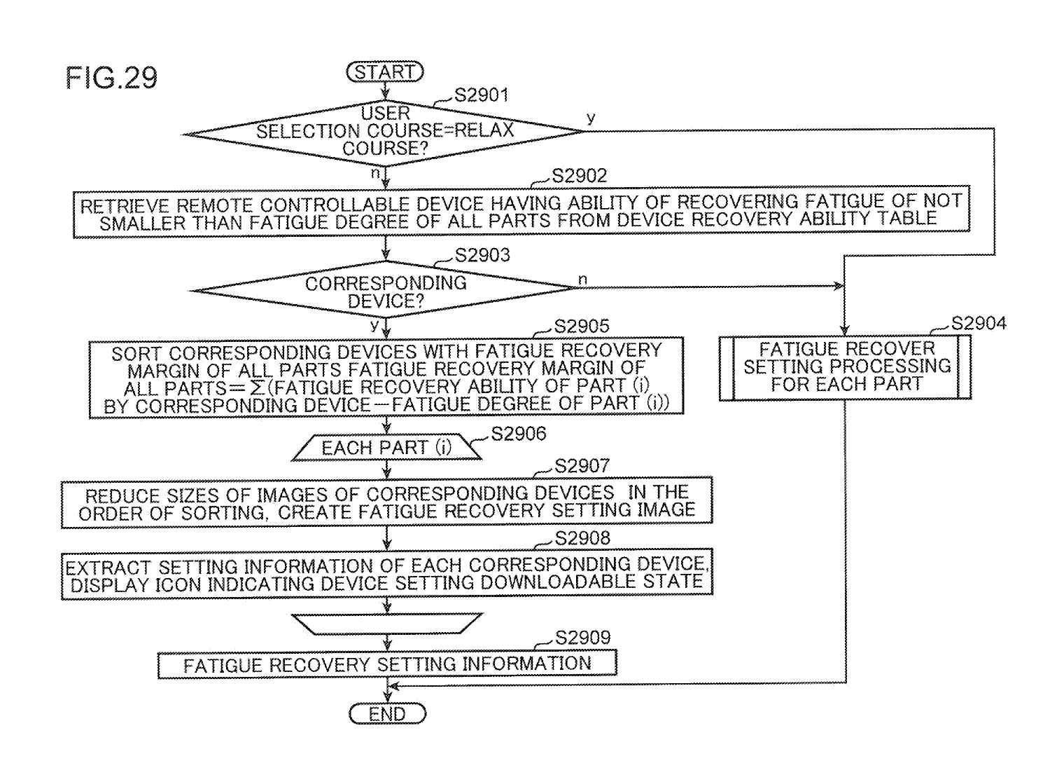

FIG. 29 is a flowchart illustrating an example of processing to be performed by the setting information generating unit 2828. First of all, when a course selection by the user indicating that the user does not want to use the remote controllable device 2803 for a long time for recovery from fatigue (n in Step S2901), the device ability table 240A is referred to, and one or more remote controllable devices 2803 having the fatigue recovery ability capable of recovering from fatigue of not smaller than the fatigue degree of the user is retrieved for each of all the parts (in Step S2902).

As a result of Step S2902, when a corresponding remote controllable device 2803 (corresponding device) is not detected (n in Step S2903), the processing is proceeded to fatigue recovery setting processing for each part (in Step S2904). On the other hand, when a corresponding device is detected (y in Step S2903), the processing is proceeded to Step S2905.

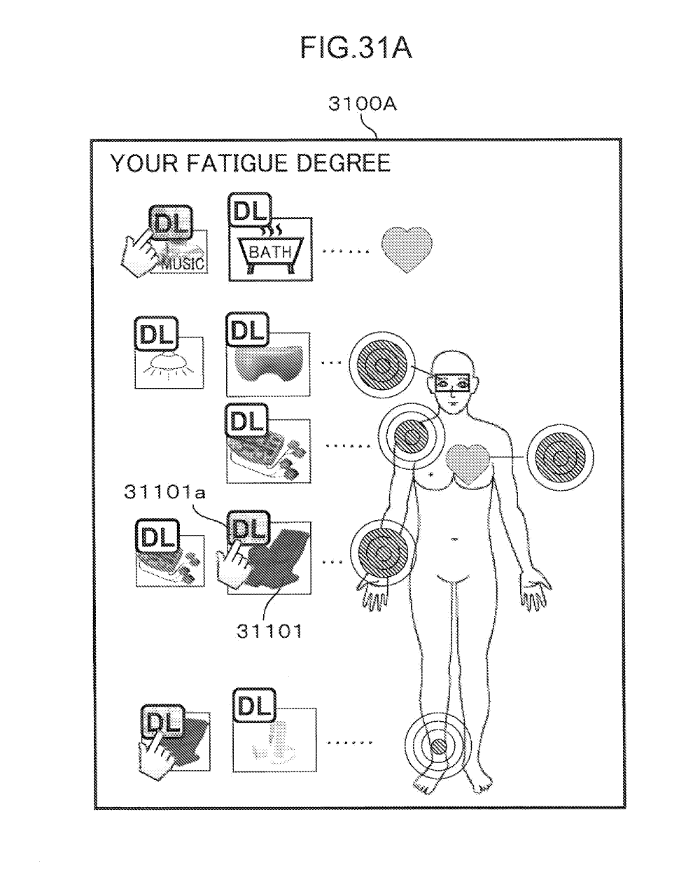

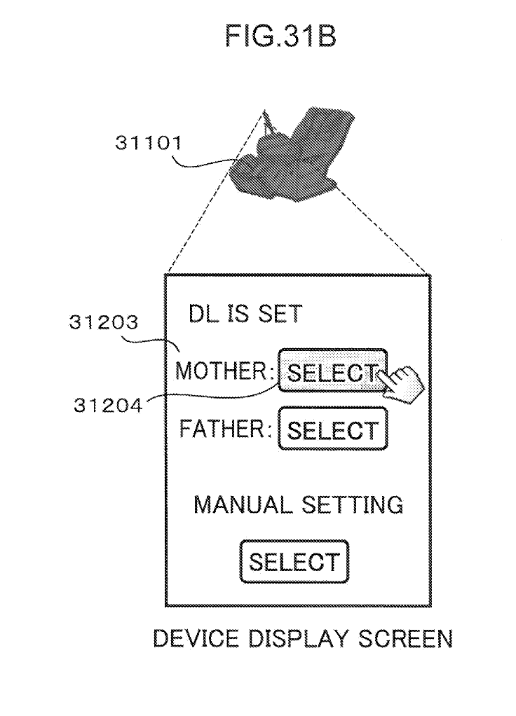

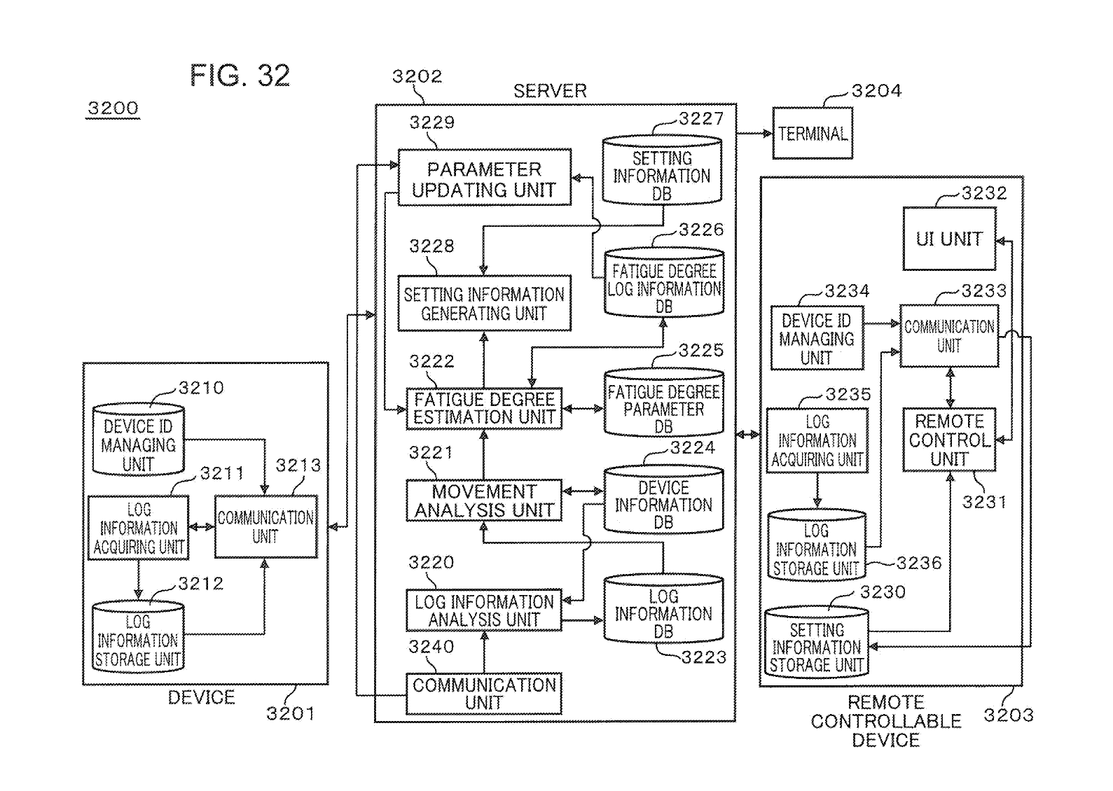

The processing from Steps S2905 to S2907 is substantially the same as the processing from Steps S2504 to S2506 in FIG. 25. In the embodiment, however, in Step S2907, a setting image 3100A illustrated in FIG. 31A is created.

In Step S2908, setting information suitable for the fatigue degree of the user is extracted from the setting table 240B with respect to each of the corresponding devices arranged in the setting image 3100A. The setting information extracting processing may be the processing as described in Step S2507 in FIG. 25. In Step S2908, an icon indicating a setting information downloadable state is displayed in the vicinity of the image of the corresponding device in each of the corresponding devices.

When the processing of Step S2906 is ended for all the parts (i), the fatigue recovery setting image 3100A is transmitted to the terminal 2804 of the user as fatigue recovery setting information (in Step S2909), and the processing is ended.

On the other hand, in Step S2901, when a course selection indicating that the user wants to use the device for a long time for recovery from fatigue is input (y in Step S2901), or in Step S2903, when it is judged that there is no corresponding device (n in Step S2903), the processing is proceeded to Step S2904.

<Fatigue Recovery Setting Processing for Each Part>

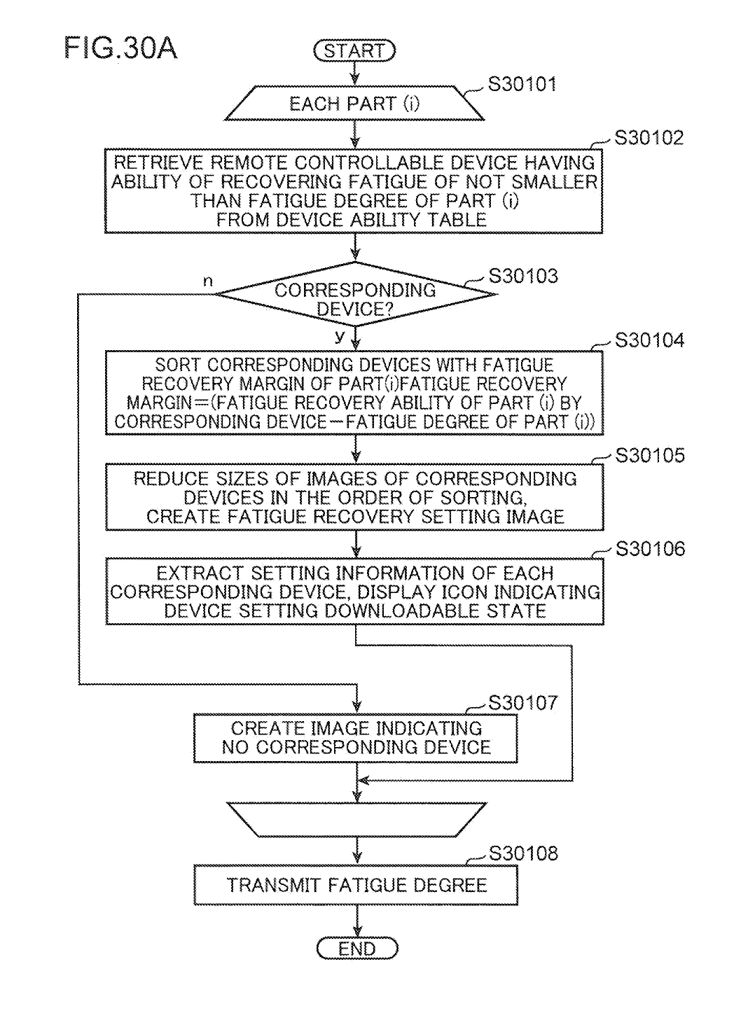

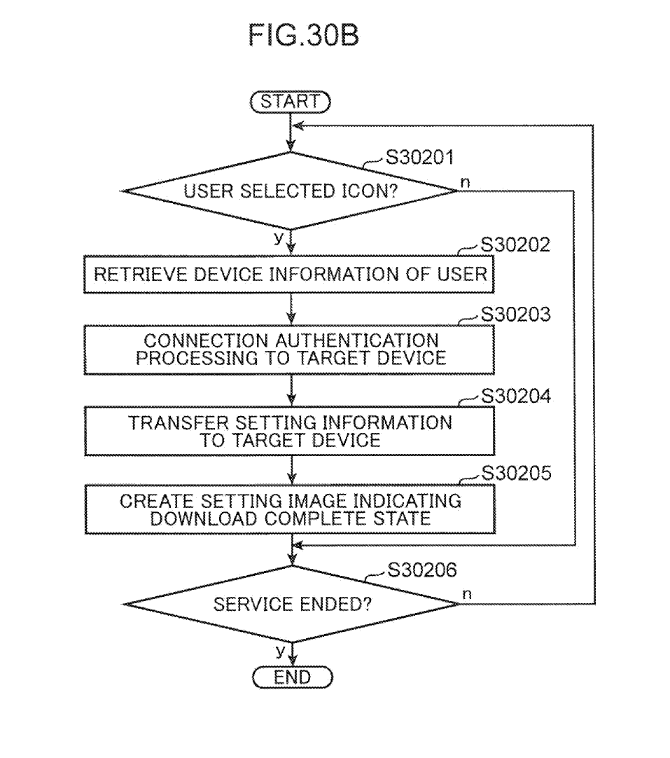

FIG. 30A is a flowchart illustrating an example of the fatigue recovery setting processing for each part (Step S2904 in FIG. 29).

The processing from Steps S30101 to S30105 is substantially the same as the processing from Steps S26101 to S26105 in FIG. 26A when the fatigue recovery devices are read as the remote controllable devices 2803. However, in Step S30105, the fatigue recovery setting image 3100A is created, in place of a fatigue recovery tip image.

In Step S30106, setting information suitable for the fatigue degree of the user is extracted from the setting table 240B with respect to each of the corresponding devices arranged in the fatigue recovery setting image 3100A. In Step S30106, an icon indicating a setting information downloadable state is displayed in the vicinity of the image of the corresponding device in each of the corresponding devices.

On the other hand, in Step S30103, when there is no corresponding device (n in Step S30103), an image or a text image indicating no remote controllable device 2803 capable of recovering from fatigue is created (in Step S30107).

When the processing of Step S30101 is ended for all the parts (i), the created fatigue recovery setting image 3100A or an image indicating no remote controllable device 2803 capable of recovering from fatigue is transmitted to the terminal 2804 of the user, as fatigue recovery setting information (in Step S30108), and the processing is ended.

<UI Processing Flow>

FIG. 30B is a flowchart illustrating an example of the UI processing in the fatigue recovery system in the fourth embodiment of the invention. Further, FIG. 31A is a diagram illustrating an example of the fatigue recovery setting image 3100A in the fatigue degree estimating system according to the fourth embodiment of the invention. Further, FIG. 31B is a diagram illustrating an example of a UI image 31203 of a massage chair 31101 owned by the user.

As illustrated in FIG. 31A, as well as in FIG. 16, in the fatigue recovery setting image 3100A, a fatigue degree of each part and a fatigue degree of a mental part are indicated by the size of a circle. Further, as well as the fatigue recovery tip image 2700 illustrated in FIG. 27, in the fatigue recovery setting image 3100A, images of the remote controllable devices 2803 capable of recovering from fatigue of each part are displayed in the order of decreasing the fatigue recovery effect in association with each part. Further, an icon 31101a indicated as “DL” is displayed in the vicinity of each of the remote controllable devices 2803. The icon 31101a is an icon through which the user is allowed to download setting information of a remote controllable device 2803 suitable for recovery from fatigue of the intended part.

In the example of FIG. 31A, an image of the massage chair 31101 is displayed as a remote controllable device 2803 capable of recovering from fatigue of the right arm. When the icon 31101a representing the massage chair 31101 is selected by the user, setting information on the massage chair 31101 suitable for recovery from fatigue of the user's right arm is downloaded from the server 2802 to the massage chair 31101.

When the setting information is downloaded to the massage chair 31101, as illustrated in FIG. 31B, a selection button 31204 for allowing the user to set the downloaded setting information (DL setting) in the massage chair 31101 is added on a UI image 31203 of the massage chair 31101. In the example of FIG. 31B, mother and father download their setting information in the massage chair 31101. Accordingly, selection buttons 31204 of mother and father are displayed individually. When mother as the user selects her selection button 31204, the massage chair 31101 is moved by the setting information downloaded from the server 2802. In this way, the massage chair 31101 is moved by the setting information suitable for recovery from fatigue of the mother's right arm.

In the following, a flowchart relating to the UI processing illustrated in FIG. 30B is described referring to FIG. 31A. First of all, when the user selects the icon 31101a of a remote controllable device 2803 (target device) from among the remote controllable devices 2803 displayed in a row in association with each part in FIG. 31A (y in Step S30201), device information of the user is retrieved (in Step S31202). Subsequently, connection authentication processing (Step S30203) to the target device is performed, and setting information on the target device suitable for recovery from fatigue, which is extracted in Step S30106 in FIG. 30A, is transferred to the target device (in Step S30204). Subsequently, the fatigue recovery setting image 3100A indicating a download complete state is created, and is transmitted to the terminal 2804 of the user (in Step S30205). In the example of FIG. 31A, the color of the icon 31101a displayed as “DL” is changed from a dotted state to a white color state, and a download complete state is indicated by deletion of the hand icon.

In Step S30201, when the user's operation is not input (n in Step S30201), the processing is proceeded to Step S30206. In Step S30206, when the user's operation of ending the service by the fatigue degree estimating system is input (y in Step S30206), the processing is ended. When the user's operation of ending the service is not input (n in step S30206), the processing returns to Step S30201, and the UI processing is continued.

As described above, in the fatigue degree estimating system according to the fourth embodiment, a remote controllable device 2803 suitable for recovery from fatigue of the user is selected from among the remote controllable devices 2803 owned by the user. When the user inputs an instruction of downloading setting information of the selected remote controllable device 2803, which is suitable for recovery from fatigue of the user, the setting information is downloaded to the remote controllable device 2803. In this way, allowing the user to select the downloaded setting information makes it possible to move the remote controllable device 2803 at a mode suitable for recovery from fatigue. This allows for the user to recover from fatigue without a cumbersome operation.

### Fifth Embodiment

A fatigue degree estimating system according to the fifth embodiment has a feature such that fatigue of the user is learned, with use of log information indicating a user's operation of a remote controllable device when the remote controllable device is remote controlled, and the fatigue degree of the user is estimated.

<System Configuration of Fifth Embodiment>

FIG. 32 is a block diagram illustrating an example of a configuration of a fatigue degree estimating system 3200 according to the fifth embodiment of the invention.

As illustrated in FIG. 32, the fatigue degree estimating system 3200 is provided with a device 3201, a server 3202, a remote controllable device 3203, and a terminal 3204.

The device 3201 and the terminal 3204 have substantially the same configuration as the device 601 and the terminal 603 in the first embodiment.

The remote controllable device 3203 is further provided with a device ID managing unit 3234, a log information acquiring unit 3235, and a log information storage unit 3236 in addition to the element blocks in the remote controllable device 2803 in the fourth embodiment. The element blocks in the remote controllable device 3203 other than the above are the same as the element blocks in the remote controllable device 2803 having the same reference numerals.

The device ID managing unit 3234 is constituted of e.g. a non-volatile memory such as a flash memory or a HDD (Hard Disk Drive), and is configured to store a unique identifier for individually identifying the remote controllable device 3203.

The log information acquiring unit 3235 is constituted of a dedicated hardware circuit or software, and is configured to acquire log information including an operation log of the user with respect to the remote controllable device 3203 and a state log of the remote controllable device 3203. In this example, the log information acquiring unit 3235 is configured to map between a user's input operation detected by the UI unit 3232, and a user's identifier, and store the mapping data in the log information storage unit 3236 as log information. Further, the log information acquiring unit 3235 is configured to store a state of the remote controllable device 3203 set by the remote control unit 3231 in response to user's input in the log information storage unit 3236, as log information.

Examples of the log information are a code number of a button in the remote controllable device 3203 pressed by the user, a function identifier representing the selection function of the remote controllable device 3203 selected by the user, and a use time of the remote controllable device 3203 by the user.

The log information storage unit 3236 is constituted of e.g. a volatile memory, and is configured to store log information acquired by the log information acquiring unit 3235.

When an access request to the log information of the remote controllable device 3203 designated by the identifier of a user is received from the setting information generating unit 2828 in the server 2802, the communication unit 3233 reads the log information of the user from the log information storage unit 3236, and transmits the read log information to the server 3202 by mapping between the log information and the unique identifier of the designated remote controllable device 3202 stored in the device ID managing unit 3234.

The server 3202 is further provided with a parameter updating unit 3229 in addition to the element blocks in the server 2802 in the fourth embodiment. The element blocks in the server 3202 other than the above are the same as the element blocks in the server 2802 having the same reference numerals.

The parameter updating unit 3229 is constituted of a software or a dedicated hardware circuit, for instance, and is configured to learn the fatigue of the user, with use of log information indicating a user's input operation with respect to the remote controllable device 3203, when the remote controllable device 3203 is remote controlled by setting information generated by a setting information generating unit 3228, and update the weighting coefficient of a calculation parameter with use of a learning result. Specifically, the parameter updating unit 3229 is configured to determine that the fatigue degree of a specific part is estimated low when an additional operation of recovering from fatigue of the specific part is input by a user with respect to the remote controllable device 3203, and update the weighting coefficient of the calculation parameter in such a manner that the fatigue degree of the specific part increases. On the other hand, the parameter updating unit 3229 is configured to determine that the fatigue degree of a user is estimated high in response to a user's operation of suspending the movement of the remote controllable device 3203, and update the weighting coefficient of the calculation parameter in such a manner that the fatigue degree of the part which is estimated to be exhausted decreases.

Subsequently, the parameter updating unit 3229 stores the identifier of the user and the corrected weighing coefficient in association with each other in a fatigue degree parameter DB 3225. Thus, a fatigue degree estimation unit 3222 estimates the fatigue degree of the user, with use of the weighting coefficient updated by the parameter updating unit 3229. This makes it possible to estimate the fatigue degree of the user.

<Processing of Updating Calculation Parameter>

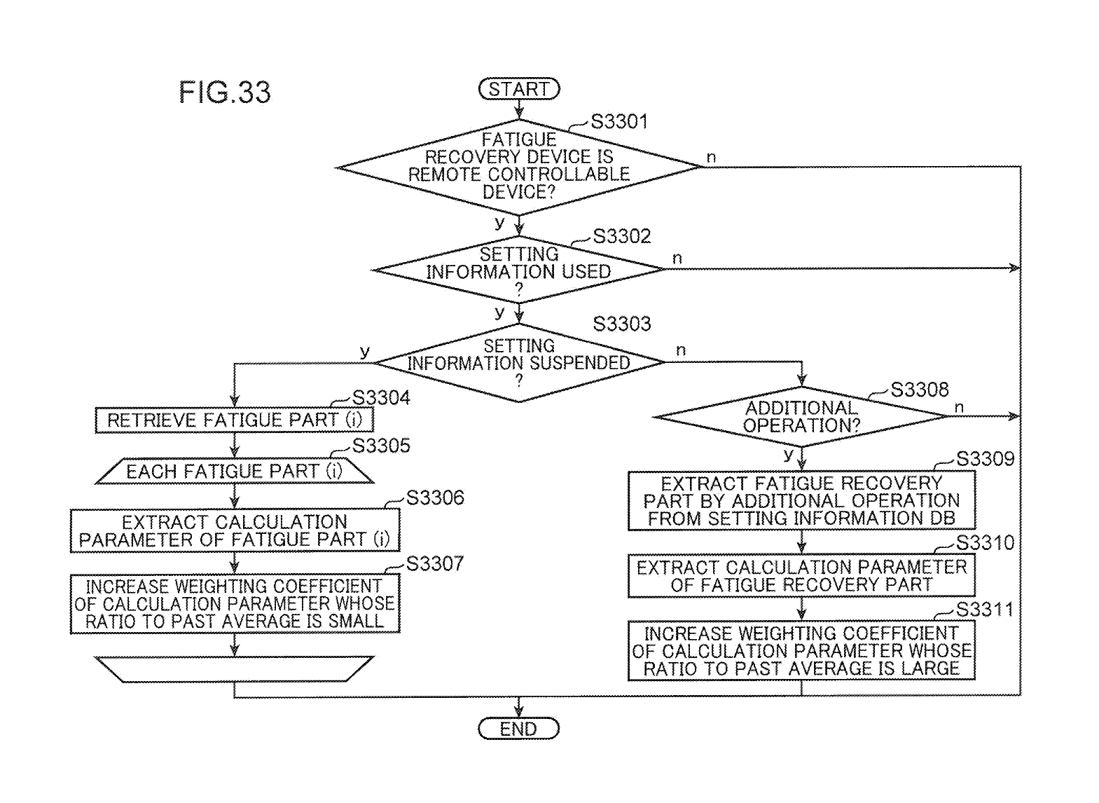

FIG. 33 is a flowchart illustrating an example of processing to be performed by the parameter updating unit 3229. First of all, the device ID managing unit 3234 is accessed to, and it is determined whether the fatigue recovery device to be used by the user is the remote controllable device 2803 (in Step S3301). When the fatigue recovery device is not the remote controllable device 3203 (n in Step S3301), the processing is ended. On the other hand, when the fatigue recovery device is the remote controllable device 3203 (y in Step S3301), the processing is proceeded to Step S3302. In this example, judgment as to whether the fatigue recovery device is the remote controllable device 3203 is performed by determining whether the unique identifier of the fatigue recovery device has a predetermined symbol string indicating that the fatigue recovery device is the remote controllable device 3203.

When the user does not use setting information suitable for recovery from fatigue, which is set by the setting information generating unit 3228 (n in Step S3302), the processing is ended. On the other hand, when the user uses the setting information (y in Step S3302), the processing is proceeded to Step S3303.

In this example, judgment as to whether setting information is used is performed by e.g. determining whether the selection button 31204 displayed on the UI image 31203 in FIG. 31B is selected by the user.

As a result of determination in Step S3302, when the setting information is not used (n in Step S3302), the processing is ended. On the other hand, when the setting information is used (y in Step S3302), it is determined whether the movement of the remote controllable device 3203 operated in accordance with the setting information is suspended (in Step S3303).

In this example, the parameter updating unit 3229 may determine whether the movement of the remote controllable device 3203 is suspended by accessing to the log information storage unit 3236 in the remote controllable device 3203 and by referring to the log information of the user.

When the movement of the remote controllable device 3203 is suspended (y in Step S3303), a part (fatigue part) whose fatigue degree is not zero is retrieved from a fatigue degree log information DB 3226 (in Step S3304).

Subsequently, the processing from Steps S3305 to S33307 is repeated for each fatigue part (i), and the processing is ended.

Calculation parameters of a fatigue part (i) are extracted from the fatigue degree log information DB 3226 (in Step S3306). For instance, in the example of FIG. 15D, assuming that the fatigue part (i) is the arm (dominant hand), the calculation parameters P2 to P7, P9, and P10 are extracted

Subsequently, the weighting coefficient of a calculation parameter whose ratio to past average is small is increased, and the weighting coefficient of a calculation parameter whose ratio to past average is large is decreased, out of the extracted calculation parameters (in Step S3307). In the aforementioned example on the arm (dominant hand), it is assumed that the ratio to past average is small with respect to the calculation parameters P2 to P7, and the ratio to past average is large with respect to the calculation parameters P9 and P10. In this case, the weighting coefficients of the calculation parameters P2 to P7 are increased by a predetermined value, and the weighting coefficients of the calculation parameters P9 and P10 are decreased so that the sum of the calculation parameters P2 to P7, P9, and P10 is equal to each other before and after the updating.

In this example, determination as to whether the ratio of calculation parameters to past average is large may be performed such that the ratio is large when a calculation parameter on this day is larger than the past average of the calculation parameter. For instance, assuming that the calculation parameter P2 indicating a working time required for the user to put the laundry in a washing machine is 6 minutes on this day and 4 minutes as the past average, the ratio to past average may be determined to be large.

The updated calculation parameter is stored in the fatigue degree parameter DB 3225 in association with the identifier of the user.

When the processing from Steps S3306 to S3307 is ended for all the fatigue parts (i), the processing of FIG. 33 is ended.

On the other hand, when the movement of the remote controllable device 3203 is not suspended (n in Step S3303), after the movement of the remote controllable device 3203 in the setting information is ended, it is determined whether the user has input an additional operation with respect to the remote controllable device 3203 (in Step S3308). The parameter updating unit 3229 may determine whether an additional operation has been input by accessing to the log information storage unit 3236, and analyzing the log information of the user.

When an additional operation is not input (n in Step S3308), the processing is ended. On the other hand, when an additional operation is input (y in Step S3308), a fatigue recovery part by the additional operation is extracted from a setting information DB 3227 (in Step S3309). In this example, a fatigue recovery part by an additional operation indicates a part for which recovery from fatigue is performed by the user by moving the remote controllable device 3203. In the example of the massage chair in FIG. 24B, assuming that the setting information C is input as an additional operation, “LEFT SHOULDER” and “RIGHT SHOULDER” whose fatigue recovery ability is defined to be larger than zero are extracted as a fatigue recovery part.

Subsequently, a calculation parameter of the extracted fatigue recovery part is extracted from the fatigue degree log information DB 3226 (in Step S3310). In the example of FIG. 15D, assuming that “SHOULDERS” is extracted as a fatigue recovery part, the calculation parameters P2 to P7, P9, and P10 are extracted.

Subsequently, the weighting coefficient of a calculation parameter whose ratio to past average is large is increased, and the weighting coefficient of a calculation parameter whose ratio to past average is small is decreased, out of the extracted calculation parameters (in Step S3311).

In the aforementioned example on “SHOULDERS”, it is assumed that the ratio of the calculation parameters P2 to P7 to past average is large, and the ratio of the calculation parameters P9 and P10 to past average is small. In this case, the weighting coefficients of the calculation parameters P2 to P7 are increased by a predetermined value, and the weighting coefficients of the calculation parameters P9 and P10 are decreased so that the sum of the calculation parameters P2 to P7, P9, and P10 is equal to each other before and after the updating. The processing of Step S3311 may be performed for each fatigue recovery part, as far as it is possible to extract two or more fatigue recovery parts in Step S3309.

Further, the flowchart of FIG. 33 may be periodically executed, or may be executed each time the number of log information items to be stored in the log information storage unit 3236 reaches a predetermined number.

### <Movement Example>

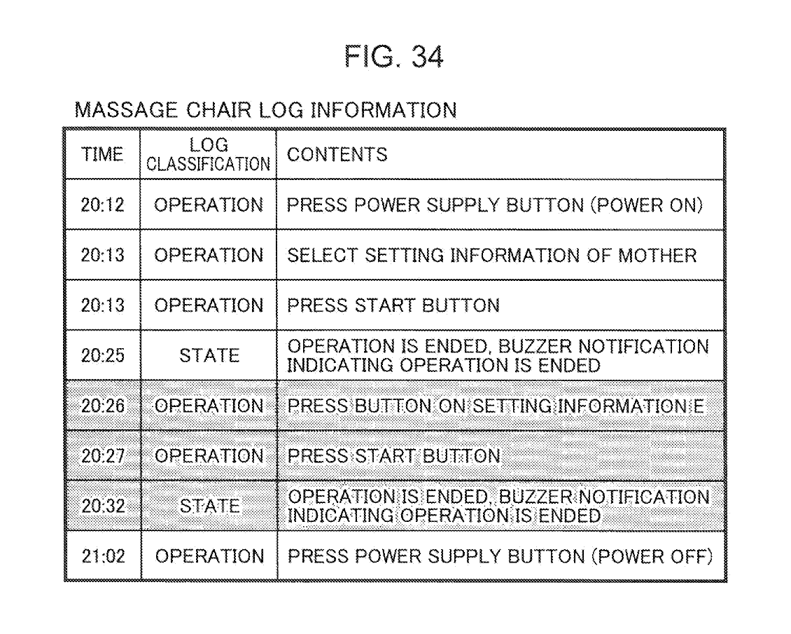

FIG. 34 is a diagram illustrating an example of log information when the massage chair as the remote controllable device 3203 is used by the user after the user has done the household chores indicated by the washing machine log information and the cleaner log information in FIG. 13. The log information is the information that has been acquired by the log information acquiring unit 3235 and stored in the log information storage unit 3236.

The user, who is mother, selects downloaded setting information suitable for recovery from her fatigue from the log information illustrated in FIG. 34 (time=20:13), massages herself (time=20:25), presses the button designating the setting information E (time=20:26), and additionally massages herself. In the example of FIG. 24B, the setting information E is setting effective for recovery from fatigue of her left arm. Accordingly, the user's left arm is extracted as a fatigue recovery part, it is judged that the fatigue recovery ability represented by the setting information E with respect to the fatigue recovery part is insufficient for the user, and the fatigue degree of the left arm is increased. In the example of FIG. 15D, the calculation parameters on the fatigue degrees of the left arm, which is the non-dominant hand, are P2 (working time required for the user to put the laundry in the washing machine). P3 (weight of the laundry before washing), P4 (number of pieces of the laundry), P5 (operation time of the cleaner), P6 (operation time of the cleaner in high place by non-dominant hand), P7 (weight of the laundry after dewatering), P9 (working time required for the user to take out the laundry), and P10 (tidying up time).

Further, in the example of FIG. 13, the ratios of these calculation parameters to past average are respectively 1.5 (P2). 0.83 (P3). 0.6 (P4), 1.5 (P5), 1 (P6), 0.78 (P7), 1.25 (P9), and 1.25 (P10). Among these, the calculation parameters P2, P9, and P10 have a large ratio of a calculation parameter to past average. In this case, the weighting coefficients of the calculation P2, P9, and P10 are increased by a predetermined value, and the weighting coefficients of the other calculation parameters are decreased so that the sum of the calculation parameters P2 to P7, P9, and P8 is equal to each other before and after the updating. This increases the fatigue degree of the left arm, whose fatigue degree is estimated low, and reflects a fatigue degree estimation thereafter. In this way, setting information suitable for the user's left arm is set thereafter. This makes it possible to guide the user for recovery from fatigue.

As described above, in the fatigue degree estimating system 3200 according to the fifth embodiment, fatigue of the user is actually learned with use of log information of the remote controllable device 3203 by the user for use in recovery from fatigue. This is advantageous in effectively recovering from fatigue of the user.

### Application Examples

The invention is applicable to the following services based on an estimated fatigue degree.

introduction of accommodations, recreation centers, nap facilities, treatment places, saunas, beauty salons, restaurants, and amusement centers, and proposal of optimal services therein;

proposal of meal menus prompting recovery from fatigue;

proposal of prescriptions such as medicines, and introduction of drug stores and medical institutions; and

Internet-based word-of-mouth information and introduction of communities having a high relevancy to fatigue conditions

The techniques described in the above aspects may be implemented by the following cloud service types, for instance. The service types to be implemented by the techniques described in the above aspects, however, are not limited to the following.

(Service Type 1: A Service Provided by a Datacenter of the Applicant's Company)

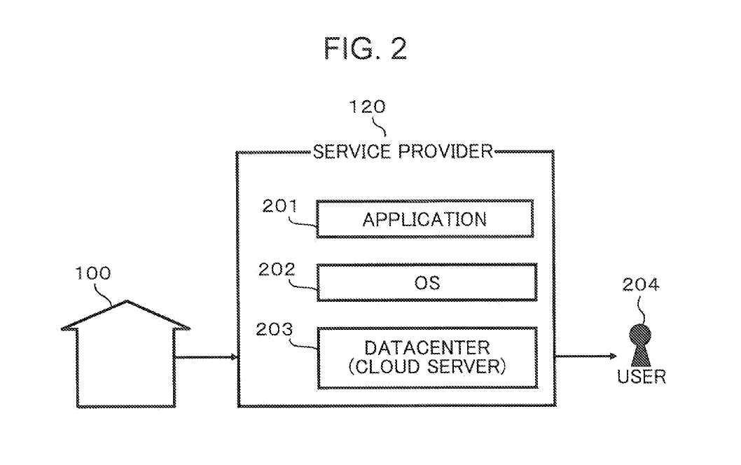

FIG. 2 illustrates service type 1 (a service provided by a datacenter of the applicant's company). In this type, a service provider 120 acquires information from a group 100, and provides service to a user 204. In this type, the service provider 120 has a function of a datacenter operating company. Specifically, the service provider 120 owns a cloud server 111 (a datacenter 203) which manages big data. Therefore, actually, a datacenter operating company does not exist.

In this type, the service provider 120 is configured to operate and manage the datacenter 203 (the cloud server 111). Further, the service provider 120 is configured to manage an OS 202 and an application 201. The service provider 120 is configured to provide the user 204 with services with use of the OS 202 and the application 201 to be managed by the service provider 120.

(Service Type 2: A Service Utilizing IaaS)

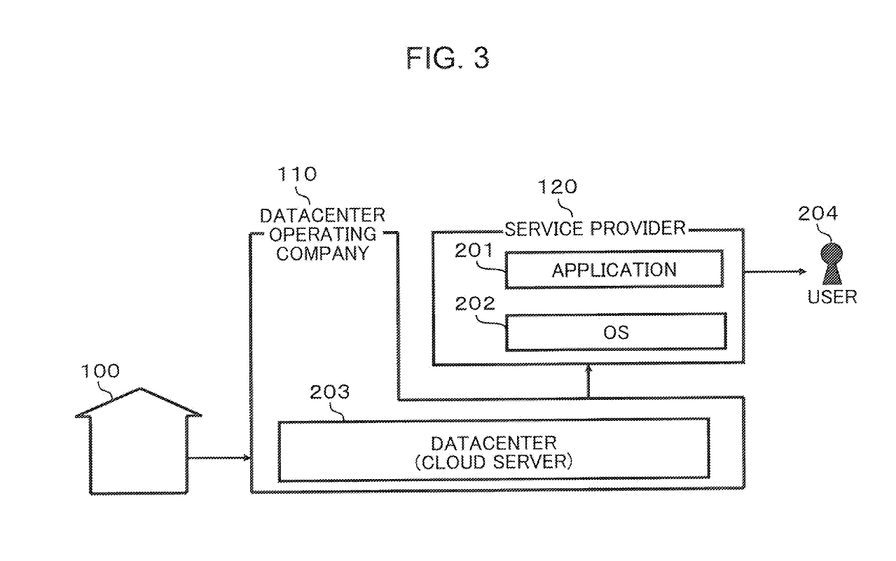

FIG. 3 illustrates service type 2 (a service utilizing IaaS). IaaS stands for Infrastructure as a Service. IaaS is a service providing model configured to provide a foundation, based on which a computer system is constructed and operated, as services via the Internet.

In this type, a datacenter operating company 110 is configured to operate and manage a datacenter 203 (a cloud server 111). Further, a service provider 120 is configured to manage an OS 202 and an application 201. The service provider 120 is configured to provide the user 204 with services with use of the OS 202 and the application 201 to be managed by the service provider 120.

(Service Type 3: A Service Utilizing PaaS)

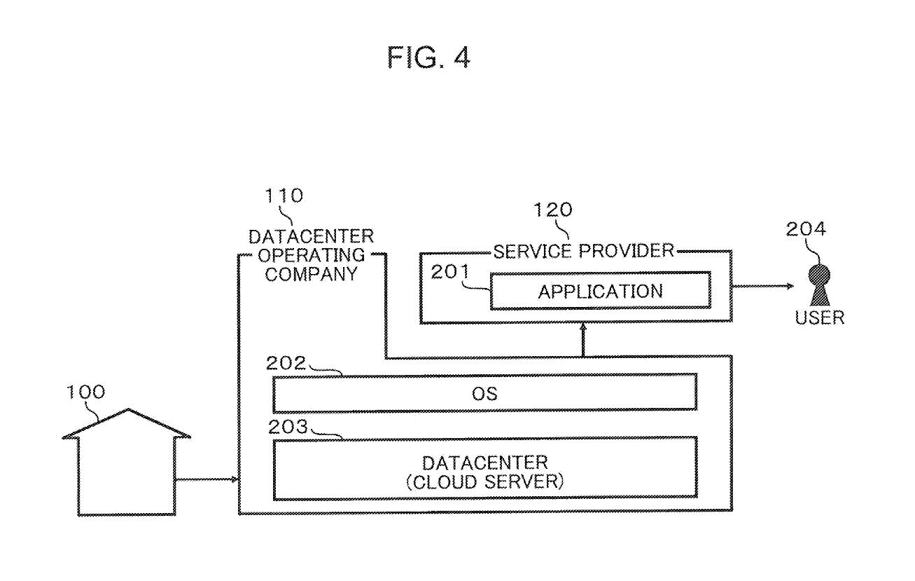

FIG. 4 illustrates service type 3 (a service utilizing PaaS). PaaS stands for Platform as a Service. PaaS is a cloud service providing model configured to provide a platform, based on which a software is constructed and operated, as services via the Internet.

In this type, a datacenter operating company 110 is configured to manage an OS 202, and operate and manage a datacenter 203 (a cloud server 111). Further, a service provider 120 is configured to manage an application 201. The service provider 120 is configured to provide the user 204 with services with use of the OS 202 to be managed by the datacenter operating company and the application 201 to be managed by the service provider 120.

(Service Type 4: A Service Utilizing SaaS)

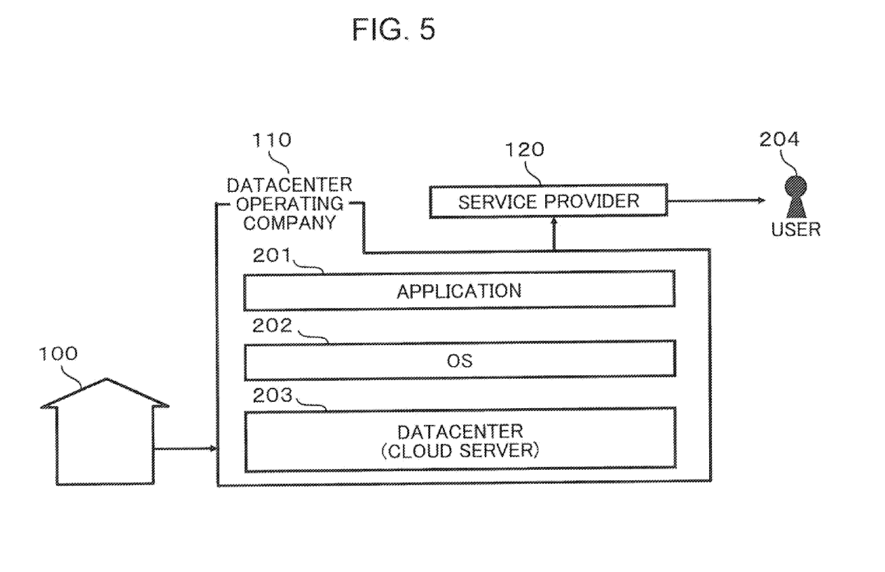

FIG. 5 illustrates service type 4 (a service utilizing SaaS). SaaS stands for Software as a Service. A service utilizing SaaS is e.g. a cloud service providing model having a function, with which a company or a person (user) who does not own a datacenter 203 (a cloud server 111) is allowed to use an application provided by a platform provider who owns a datacenter 203 (a cloud server 111) via a network such as the Internet.

In this type, a datacenter operating company 110 is configured to manage an application 201 and an OS 202, and operate and mange a datacenter 203 (the cloud server 111). Further, a service provider 120 is configured to provide the user 204 with services, with use of the OS 202 and the application 201 to be managed by the datacenter operating company 110.

As described above, in any of the service types, the service provider 120 is configured to provide services. Further, for instance, a service provider 120 or a datacenter operating company 110 may develop an OS 202, an application 201, or a database for big data by themselves, or may outsource the development to a third party.

### INDUSTRIAL APPLICABILITY

The fatigue degree estimating system according to an aspect of the invention is useful as a system for recovering from fatigue resulting from doing household chores by utilizing information such as an operation history and a movement history of household electrical devices, and AV equipment, for instance.

1. A fatigue degree estimating method for use in a fatigue degree estimating system for estimating a fatigue degree of a user in doing household chores, comprising:
a movement analyzing step of analyzing log information including an operation log of the user with respect to a device and a state log of the device for analyzing a direct operation by the user with respect to the device, and movement of the user before and after the direct operation; and
a fatigue degree estimating step of estimating a fatigue degree of each part of the user and of a mental part of the user by the analyzed direct operation and the analyzed movement before and after the direct operation.

2. The fatigue degree estimating method according to claim 1, further comprising:
a message notifying step of generating a message of soothing, appreciation, or praise in accordance with the estimated fatigue degree, and notifying at least one of the user and a person related to the user of the message.

3. The fatigue degree estimating method according to claim 1, further comprising:
a tip message notifying step of selecting a fatigue recovery device suitable for recovery of the estimated fatigue degree, with use of device ability information in which a fatigue recovery ability of the each part of the user is defined individually with respect to one or more fatigue recovery devices owned by the user, generating a message proposing a fatigue recovery tip utilizing the selected fatigue recovery device, with use of setting information, of the selected fatigue recovery device, which defines setting contents suitable for the fatigue degree of the each part of the user, and notifying the user of the message.

4. The fatigue degree estimating method according to claim 1, further comprising:
a setting information generating step of selecting a remote controllable device suitable for recovery of the estimated fatigue degree, with use of device ability information in which a recovery ability of the each part of the user is defined individually with respect to one or more remote controllable devices, selecting setting information suitable for recovery of the estimated fatigue degree, from among one or more setting information in which setting contents of the remote controllable device suitable for a fatigue degree of the each part of the user is defined, and transmitting the selected setting information to the remote controllable device, the remote controllable devices being remote controllable fatigue recovery devices owned by the user.

5. The fatigue degree estimating method according to claim 4, further comprising:
a learning step of learning fatigue of the user, with use of log information indicating an operation of the remote controllable device by the user, when the remote controllable device is moved with use of the set setting information.

6. The fatigue degree estimating method according to claim 1, wherein
the movement analyzing step includes specifying, out of the log information, log information for which a calculation parameter is set, the specified log information relating to the direct operation and to the movement before and after the direct operation, and
the fatigue degree estimating step includes setting a calculation parameter in accordance with contents of the log information with respect to the specified log information, multiplying the set calculation parameter by a weighting coefficient in accordance with the mental part and the each part of the user, and summing the calculation parameter multiplied by the weighting coefficient individually for the mental part and for the each part of the user so as to calculate a fatigue degree of the mental part and of the each part of the user.

7. The fatigue degree estimating method according to claim 2, wherein
the message to be notified to the person related to the user includes an input column in which the person related to the user inputs a message to the user, and
the message notifying step includes containing the input message in the message to be notified to the user, when the message is input in the input column.

8. The fatigue degree estimating method according to claim 3, wherein
the tip message notifying step includes:
calculating a fatigue recovery margin indicating a margin of the recovery ability of each of the fatigue recovery devices with respect to the estimated fatigue degree,
specifying one or more fatigue recovery devices in the order of decreasing the fatigue recovery margin, and setting an image indicating the specified fatigue recovery device with a size corresponding to a magnitude of the fatigue recovery margin for the each part of the user, and
generating the message, while arranging the images whose sizes are set in the order of decreasing the fatigue recovery margin for the each part of the user.

9. The fatigue degree estimating method according to claim 8, wherein
the tip message notifying step includes, when an image indicating the fatigue recovery device is selected by the user, generating the message displaying setting information suitable for recovery from fatigue of the user in the selected fatigue recovery device.

10. The fatigue degree estimating method according to claim 4, wherein
the setting information generating step includes notifying, on a terminal of the user, a message displayed such that an icon for use in downloading the selected setting information is mapped to an image indicating the selected remote controllable device, and transmitting setting information corresponding to the selected icon to the remote controllable device when selection of the icon by the user is detected.

11. The fatigue degree estimating method according to claim 5, wherein
the fatigue degree is calculated with use of a weighting coefficient in accordance with the mental part and the each part of the user, and
the learning step includes updating the weighting coefficient in such a manner that a fatigue degree of a part of the user for which recovery from fatigue is performed by the remote controllable device is lowered, when log information indicating the operation indicates an operation of suspending movement of the remote controllable device with use of the setting information during the movement.

12. The fatigue degree estimating method according to claim 5, wherein
the fatigue degree is calculated with use of a weighting coefficient in accordance with the mental part and the each part of the user, and
the learning step includes updating the weighting coefficient in such a manner that a fatigue degree of a specific part of the user increases, when log information indicating the operation indicates input of an addition operation for recovery from fatigue of the specific part after movement of the remote controllable device with use of the setting information is ended.

13. A non-transitory computer-readable recording medium which stores a program which causes a computer of a fatigue degree estimating system for estimating a fatigue degree of a user in doing household chores to execute:
a movement analyzing step of analyzing log information including an operation log of the user with respect to a device and a state log of the device for analyzing a direct operation by the user with respect to the device, and movement of the user before and after the direct operation; and
a fatigue degree estimating step of estimating a fatigue degree of each part of the user and of a mental part of the user by the analyzed direct operation and the analyzed movement before and after the direct operation.

14. A method for providing a program which causes a computer of a fatigue degree estimating system for estimating a fatigue degree of a user in doing household chores to execute:
a movement analyzing step of analyzing log information including an operation log of the user with respect to a device and a state log of the device for analyzing a direct operation by the user with respect to the device, and movement of the user before and after the direct operation; and
a fatigue degree estimating step of estimating a fatigue degree of each part of the user and of a mental part of the user by the analyzed direct operation and the analyzed movement before and after the direct operation.
